(19) United States
(12) Patent Application Publication
(10) Pub. No.: US 2024/0287036 A1
(43) Pub. Date: Aug. 29, 2024

(54) COMPOUNDS USEFUL IN THE TREATMENT OR PREVENTION OF A PRMT5-MEDIATED DISORDER

(71) Applicant: ARGONAUT THERAPEUTICS LIMITED, Oxford (GB)

(72) Inventors: Nicholas LA THANGUE, Oxford, Great Britain (GB); Andrew MORLEY, Oxford, Great Britain (GB); Shonagh MUNRO, Oxford, Great Britain (GB)

(73) Assignee: ARGONAUT THERAPEUTICS LIMITED, Oxford (GB)

(21) Appl. No.: 18/568,813

(22) PCT Filed: Jun. 10, 2022
(86) PCT No.: PCT/GB2022/051462

§ 371 Date: Dec. 9, 2023

(30) Foreign Application Priority Data
Jun. 11, 2021 (GB) 2108383.7

(51) Int. Cl. C07D 401/14; A61K 31/4725; A61K 31/497; A61K 31/501; A61K 31/506; A61K 31/55

(52) U.S. Cl. CPC C07D 401/14; A61K 31/4725; A61K 31/497; A61K 31/501; A61K 31/506; A61K 31/55

(57) ABSTRACT

The present disclosure relates to compounds suitable for the inhibition of protein arginine methyl-transferase (PRMT), in particular PRMT5. These compounds may be for use as therapeutic agents, in particular, agents for use in the treatment and/or prevention of proliferative diseases, such as cancer.

### FIELD OF THE INVENTION

The invention relates to compounds suitable for the inhibition of protein arginine methyl-transferase (PRMT), in particular PRMT5. These compounds may be for use as therapeutic agents, in particular, agents for use in the treatment and/or prevention of proliferative diseases, such as cancer.

### BACKGROUND

The transition from G1 into S phase of the cell cycle is tightly regulated in normal cells, but universally deregulated in tumour cells. The pathway involves the retinoblastoma tumour suppressor (pRb) protein, which acts to negatively regulate the G1 to S phase transition through its key target, the E2F family of transcription factors. E2F transcription factors control the expression of a variety of genes that are intimately connected with cell proliferation and cell death, including many involved with DNA synthesis. In tumour cells, normal regulation of E2F is lost (due to oncogenic mutation in the Rb gene or deregulation of Rb activity through other oncogenically-relevant mechanisms), liberating E2F, which subsequently drives cells into S phase and enables cell division to occur. The first member of the family, E2F1, is an important regulator of cell fate. E2F1 both promotes cell proliferation and also causes the opposing outcome, namely apoptosis (cell death).

The protein arginine methyl transferase PRMT5 is elevated in many human malignancies, including lymphomas, lung cancer, breast cancer and colorectal cancer, and its expression level correlates with poor disease prognosis. It is one of the major protein PRMTs in mammalian cells, exhibiting roles in cell death, cell-cycle progression, cell growth and cell proliferation. From the perspective of cancer drug discovery, arginine methylation of E2F1 by PRMT5 is responsible for keeping E2F1 in its growth stimulating mode. This occurs because arginine methylation by PRMT5 suppresses apoptosis driven by E2F-1, and thereby holds E2F-1 and cells expressing methylated E2F1 in their growing state. Thus, inhibiting PRMT5 enzyme activity provides a rational approach to reinstating tumour cell death by reactivating a physiological mechanism, dependent on E2F1 activity, which is responsible for keeping abnormal growth in check.

The relationship between PRMT5 and cancer has been studied extensively, for example, in the references cited below.

There is a need to develop compounds that reduce the expression or activity of PRMT5, particularly compounds that can be used in a clinical setting.

### SUMMARY OF THE INVENTION

The invention provides a compound of formula (1) or a deuterated form, salt, solvate, or hydrate thereof,

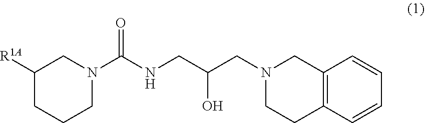

wherein:
   R^{1A} is represented by formula (A1) or (A1′),

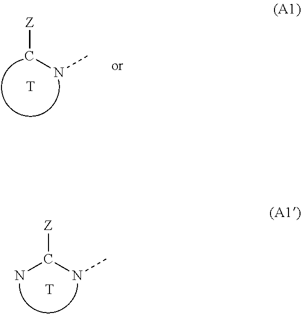

Z is ═O;
T taken together with the intervening carbon and nitrogen atoms (e.g. shown in formula (A1) and (A1′)) is selected from a monocyclic 5- to 7-membered heterocycloalkyl group, a fused bicyclic 6- to 10-membered heterocycloalkyl group and a bridged bicyclic 6- to 9-membered heterocycloalkyl group, wherein each of the monocyclic 5- to 7-membered heterocycloalkyl group, the fused bicyclic 6- to 10-membered heterocycloalkyl group and the bridged bicyclic 6- to 9-membered heterocycloalkyl group is optionally substituted with one or more R^{S1};
R^{S1} is selected from C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
R^{S2} is selected from hydroxy, halo, CN and nitro.

R^{1A} is represented by formula (A1) and (A1′) above, where the dashed bond represents the point of attachment of (A1) or (A1′) to formula (1), as shown in formula (1′) or (1″) below.

When T is an unsubstituted group, then each constituent atom of the group may be attached to hydrogen atom(s) to satisfy the correct valency of that constituent atom. For example, in formula (A1′) above, when the nitrogen atom shown has two single bonds that make up the ring represented by T, the nitrogen atom is bonded to a hydrogen atom (e.g. to ensure that the nitrogen atom is trivalent).

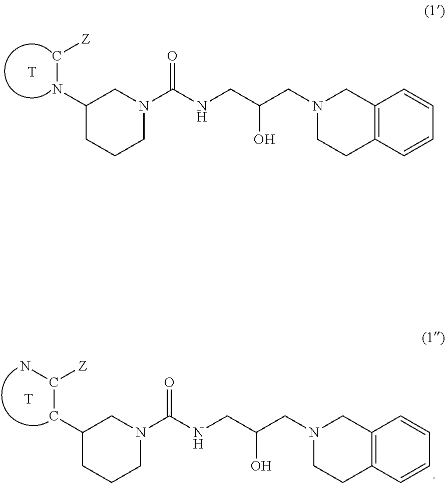

The invention also provides a pharmaceutical composition, which comprises a compound of formula (1), (1′) or (1″) as defined herein, or a deuterated form, pharmaceutically acceptable salt, hydrate or solvate thereof. The pharmaceutical composition may further comprise a pharmaceutically acceptable excipient.

The compounds of the invention are PRMT5 inhibitors and show excellent inhibitory activity in an in vitro PRMT5 assay. In addition to this in vitro activity, the compounds of the invention show excellent metabolic stability, which is typically superior to other PRMT5 inhibitors known in the art. This metabolic stability is believed to be due to one or more structural features of the compounds, include the side group represented by formula (A1) or (A1′) and the position at which it is attached to the piperidinyl ring in formula (1), (1′) or (1″).

The invention further provides a compound of formula (1), (1′) or (1″) as defined herein, or a deuterated form, a pharmaceutically acceptable salt, hydrate or solvate thereof, or a pharmaceutical composition as defined herein, for use in therapy and/or for use as a medicament.

In another aspect, the invention provides a compound of formula (1), (1′) or (1″) as defined herein, or a deuterated form, pharmaceutically acceptable salt, hydrate or solvate thereof, or a pharmaceutical composition as defined herein, for use in the treatment or prevention of a PRMT5-mediated disorder.

A further aspect of the invention provides a compound of formula (1), (1′) or (1′) as defined herein, or a deuterated form, a pharmaceutically acceptable salt, hydrate or solvate thereof, or a pharmaceutical composition as defined herein, for use in the treatment of a proliferative disorder.

In another aspect, the invention provides a compound of formula (1), (1′) or (1″) as defined herein, or a deuterated form, pharmaceutically acceptable salt, hydrate or solvate thereof, or a pharmaceutical composition as defined herein, for use in the treatment cancer.

In a further aspect, the invention provides the use of a compound of formula (1), (1′) or (1″) as defined herein, or a deuterated form, a pharmaceutically acceptable salt, hydrate or solvate thereof, in the manufacture of a medicament for the treatment or prevention of a PRMT5-mediated disorder.

In another aspect, the invention provides the use of a compound of formula (1), (1′) or (1″) as defined herein, or a deuterated form, a pharmaceutically acceptable salt, hydrate or solvate thereof, in the manufacture of a medicament for the treatment of a proliferative disorder.

A further aspect of the invention provides the use of a compound of formula (1), (1′) or (1″) as defined herein, or a deuterated form, a pharmaceutically acceptable salt, hydrate or solvate thereof, in the manufacture of a medicament for the treatment of cancer.

In another aspect, the invention provides a method of treating or preventing a PRMT5-mediated disorder, said method comprising administering to a subject in need thereof an effective amount of a compound of formula (1), (1′) or (1″) as defined herein, or a deuterated form, a pharmaceutically acceptable salt, hydrate or solvate thereof.

In another aspect, the invention provides a method of treating a proliferative disorder, said method comprising administering to a subject in need thereof an effective amount of a compound of formula (1), (1′) or (1″) as defined herein, or a deuterated form, a pharmaceutically acceptable salt, hydrate or solvate thereof.

In another aspect, the invention provides a method of treating cancer, said method comprising administering to a subject in need thereof a therapeutically effective amount of a compound of formula (1), (1′) or (1″) as defined herein, or a deuterated form, a pharmaceutically acceptable salt, hydrate or solvate thereof, or a pharmaceutical composition as defined herein.

In another aspect, the invention provides a method of inhibiting the activity of PRMT5 in vivo or in vitro. When the method is an in vivo or in vitro method, then the method may comprise contacting a cell with an effective amount of a compound of formula (1), (1′) or (1″) as defined herein, or a deuterated form, pharmaceutically acceptable salt, hydrate or solvate thereof, or a pharmaceutical composition as defined herein. When the method is an in vivo method, then additionally or alternatively the method may comprise administering to a subject in need thereof a therapeutically effective amount of a compound of formula (1), (1′) or (1″) as defined herein, or a deuterated form, a pharmaceutically acceptable salt, hydrate or solvate thereof, or a pharmaceutical composition as defined herein.

In a further aspect, the invention provides a method of altering gene expression in a cell which comprises contacting a cell with an effective amount of a compound of formula (1), (1′) or (1″) as defined herein, or a deuterated form, a pharmaceutically acceptable salt, hydrate or solvate thereof, or a pharmaceutical composition as defined herein.

In another aspect, the invention provides a combination comprising a compound of formula (1), (1′) or (1″) as defined herein, or a deuterated form, a pharmaceutically acceptable salt, hydrate or solvate thereof, and one or more additional therapeutic agents.

In a further aspect, the invention provides a pharmaceutical composition as defined herein further comprising one or more additional therapeutic agents.

Preferred, suitable, and optional features of any one particular aspect of the invention are also preferred, suitable, and optional features of any other aspect.

### DETAILED DESCRIPTION

### Definitions

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of formula (1) or (1′)” and the more general term “compounds” refer to and include any and all compounds described by and/or with reference to formula (1) or formula (1′). It should also be understood that these terms encompass all stereoisomers, i.e. cis and trans isomers, as well as optical isomers, i.e. R and S enantiomers, of such compounds and all salts thereof, in substantially pure form and/or any mixtures of the foregoing in any ratio. This understanding extends to pharmaceutical compositions and methods of treatment that employ or comprise one or more compounds of formula (1) or (1′), either by themselves or in combination with additional agents.

The various hydrocarbon-containing moieties provided herein may be described using a prefix designating the minimum and maximum number of carbon atoms in the moiety, e.g. “C_{a-b}” or “Cₐ-C_{b}”. For example, C_{a-b}alkyl indicates an alkyl moiety having the integer “a” to the integer “b” number of carbon atoms, inclusive. Certain moieties may also be described according to the minimum and maximum number of members with or without specific reference to a particular atom or overall structure. For example, the terms “a to b membered ring” or “having between a to b members” refer to a moiety having the integer “a” to the integer “b” number of atoms, inclusive.

The term “about” when used herein in conjunction with a measurable value such as, for example, an amount or a period of time and the like, is meant to encompass reasonable variations of the value, for instance, to allow for experimental error in the measurement of said value.

As used herein by themselves or in conjunction with another term or terms, “alkyl” and “alkyl group” refer to a branched or unbranched saturated hydrocarbon chain. Unless specified otherwise, an alkyl group typically contains 1-6 carbon atoms, such as 1˜4 carbon atoms or 1-3 carbon atoms, and can be substituted or unsubstituted. Representative examples include, but are not limited to, methyl, ethyl, n-propyl, i-propyl, n-butyl, i-butyl, s-butyl, t-butyl, n-pentyl, n-hexyl, isopropyl, tert-butyl, isobutyl, etc.

As used herein by themselves or in conjunction with another term or terms, “alkenyl” and “alkenyl group” refer to a branched or unbranched hydrocarbon chain containing at least one double bond. Unless specified otherwise, alkenyl groups typically contain 2-6 carbon atoms, such as 2-4 carbon atoms, and can be substituted or unsubstituted. Representative examples include, but are not limited to, ethenyl, 3-buten-1-yl, 2-ethenylbutyl, and 3-hexen-1-yl.

As used herein by themselves or in conjunction with another term or terms, “alkynyl” and “alkynyl group” refer to a branched or unbranched hydrocarbon chain containing at least one triple bond. Unless specified otherwise, alkynyl groups typically contain 2-6 carbon atoms, such as 2-4 carbon atoms, and can be substituted or unsubstituted. Representative examples include, but are not limited to, ethynyl, 3-butyn-1-yl, propynyl, 2-butyn-1-yl, and 3-pentyn-1-yl.

As used herein by themselves or in conjunction with another term or terms, “alkoxy” and “alkoxy group” refer to an alkyl group singularly bonded to oxygen (e.g. alkyl-O—). Representative examples include, but are not limited to, —OCH₃, —OCH₂CH₃, —OCH(CH₃)₂.

As used herein by themselves or in conjunction with another term or terms, “cycloalkyl” and “cycloalkyl group” refer to a non-aromatic carbocyclic ring system, that may be monocyclic, bicyclic, or tricyclic, saturated or unsaturated, and may be bridged, spiro, and/or fused. A cycloalkyl group may be substituted or unsubstituted. Unless specified otherwise, a cycloalkyl group typically contains from 3 to 12 ring atoms. In some instances, a cycloalkyl group may contain 4 to 10 ring atoms (e.g., 4 ring atoms, 5 ring atoms, 6 ring atoms, 7 ring atoms, etc.). Representative examples include, but are not limited to, cyclopropyl, cyclopropenyl, cyclobutyl, cyclobutenyl, cyclopentyl, cyclopentenyl, cyclohexyl, cyclohexenyl, norbornyl, norbornenyl, bicyclo[2.2.1]hexane, bicyclo[2.2.1]heptane, bicyclo[2.2.1]heptene, bicyclo[3.1.1]heptane, bicyclo[3.2.1]octane, bicyclo[2.2.2]octane, bicyclo[3.2.2]nonane, bicyclo[3.3.1]nonane, and bicyclo[3.3.2]decane. Suitably, cycloalkyl groups are selected from cyclopropyl, cyclobutyl, cyclopentyl and cyclohexyl groups.

As used herein by themselves or in conjunction with another term or terms, “haloalkyl” and “haloalkyl group” refer to alkyl groups in which one or more hydrogen atoms are replaced by halogen atoms. Haloalkyl includes saturated alkyl groups, but not unsaturated alkenyl and alkynyl groups. Representative examples include, but are not limited to, —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CHFCH₂CH₃ and —CHFCH₂CF₃. Haloalkyl groups can be substituted or unsubstituted. Typically, a haloalkyl group is selected from CHF₂ and CF₃, suitably CF₃.

As used herein by themselves or in conjunction with another term or terms, “halo” and “halogen” include fluoro, chloro, bromo and iodo substituents (e.g. chlorine, bromine and iodine atoms).

As used herein by themselves or in conjunction with another term or terms, “heterocycloalkyl” and “heterocycloalkyl group” refer to a non-aromatic ring system, which contains, in addition to carbon atom(s), at least one heteroatom, such as nitrogen, oxygen, sulphur or phosphorus. The heterocycloalkyl group may be fully saturated or may be partially unsaturated (i.e. contains unsaturated portions). In some instances, a heterocycloalkyl group may contain at least two or heteroatoms, which may be the same or different. Heterocycloalkyl groups can be substituted or unsubstituted. Where indicated, the heterocycloalkyl group may be monocyclic or polycyclic. When the heterocycloalkyl group is polycyclic, then it may be a fused polycyclic group, preferably a fused bicyclic group, or a bridged polycyclic group. Representative examples of monocyclic heterocycloalkyl groups include, but are not limited to, piperidin-2-one, tetrahydropyrimidin-2(1H)-one, pyridazin-3(2H)-one, tetrahydropyridazon-3(2H)-one, pyrrolidine-2-one, 3,4-dihydroxy-2H-pyrrol-2-one, 2H-pyrrol-2-one, imidazolidine-2-one, 1,3-dihydro-2H-imidazol-2-one. Representative examples of fused bicyclic heterocycloalkyl groups include, but are not limited to, 3-azabicyclo[4.1.0]heptan-2-one and octahydro-1H-cyclopenta[c]pyridin-1-one. A representative example of a bridged bicyclic heterocycloalkyl group, but not limited to, is (1R,4S)-2-azabicyclo[2.2.1]heptan-3-one.

Suitably, a heterocycloalkyl group as defined herein is a group comprising a total of one, two or three heteroatoms, where one of the heteroatoms is N and any other heteroatoms are selected from N, O and S.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

As used herein by itself or in conjunction with another term or terms, “pharmaceutical composition” refers to a composition that can be used to treat a disease, condition, or disorder in a subject, including a human.

As used herein by themselves or in conjunction with another term or terms, “subject(s)” and “patient(s)”, suitably refer to mammals, in particular humans.

As used herein by itself or in conjunction with another term or terms, “substituted” indicates that a hydrogen atom on a molecule has been replaced with a different atom or group of atoms and the atom or group of atoms replacing the hydrogen atom is a “substituent.” It should be understood that the terms “substituent”, “substituents”, “moiety”, “moieties”, “group”, or “groups” refer to substituent(s).

As used herein by themselves or in conjunction with another term or terms, “therapeutic” and “therapeutically effective amount” refer to an amount a compound, composition or medicament that (a) inhibits or causes an improvement in a particular disease, condition or disorder; (b) attenuates, ameliorates or eliminates one or more symptoms of a particular disease, condition or disorder; (c) or delays the onset of one or more symptoms of a particular disease, condition or disorder described herein. It should be understood that the terms “therapeutic” and “therapeutically effective” encompass any one of the aforementioned effects (a)-(c), either alone or in combination with any of the others (a)-(c). It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein whether by themselves or in conjunction with another term or terms, “treating”, “treated” and “treatment”, refer to and include prophylactic, ameliorative, palliative, and curative uses and results. In some embodiments, the terms “treating”, “treated”, and “treatment” refer to curative uses and results as well as uses and results that diminish or reduce the severity of a particular condition, characteristic, symptom, disorder, or disease described herein. For example, treatment can include diminishment of several symptoms of a condition or disorder or complete eradication of said condition or disorder. It should be understood that the term “prophylactic” as used herein is not absolute but rather refers to uses and results where the administration of a compound or composition diminishes the likelihood or seriousness of a condition, symptom, or disease state, and/or delays the onset of a condition, symptom, or disease state for a period of time.

As used herein, a “therapeutically active agent”, whether used alone or in conjunction with another term or terms, refers to any compound, i.e. a drug, that has been found to be useful in the treatment of a disease, disorder or condition and is not described by formula (1) or (1′). It should be understood that a therapeutically active agent may not be approved by the FDA or an equivalent foreign regulatory body.

A “therapeutically effective amount” means the amount of a compound that, when administered to a subject or patient for treating a disease, is sufficient to effect such treatment for the disease. The “therapeutically effective amount” will vary depending on the compound, the disease and its severity and the age, weight, etc., of the subject or patient to be treated.

As used herein, the term “PRMT5-mediated disorder” means any disease, disorder, or other pathological condition in which PRMT5 is known to play a role. Accordingly, the present disclosure relates to treating or lessening the severity of one or more diseases in which PRMT5 is known to play a role.

As used in the present disclosure, the term “comprises” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”. The term “consisting essentially of” may also be replaced with “consists of”.

The invention will now be further described by way of the following numbered paragraphs.
1. The invention provides a compound of formula (1) or a deuterated form, salt, solvate, or hydrate thereof,

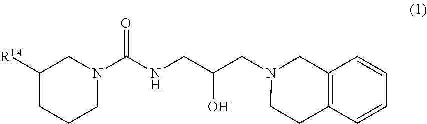

wherein:
   R^{1A} is represented by formula (A1) or (A1′),

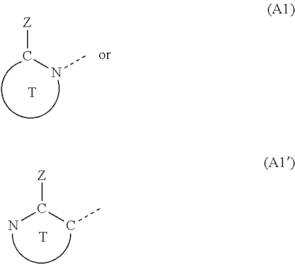

Z is ═O;
T taken together with the intervening carbon and nitrogen atoms (e.g. shown in formula (A1) and (A1′)) is selected from a monocyclic 5- to 7-membered heterocycloalkyl group, a fused bicyclic 6- to 10-membered heterocycloalkyl group and a bridged bicyclic 6- to 9-membered heterocycloalkyl group, wherein each of the monocyclic 5- to 7-membered heterocycloalkyl group, the fused bicyclic 6- to 10-membered heterocycloalkyl group and the bridged bicyclic 6- to 9-membered heterocycloalkyl group is optionally substituted with one or more R^{S1};
R^{S1} is selected from C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
R^{S2} is selected from hydroxy, halo, CN and nitro.
1A. The compound according to paragraph 1, or a deuterated form, salt, solvate or hydrate thereof, wherein R^{1A} is represented by formula (A1),

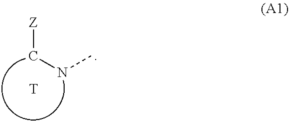

2. The compound according to paragraph 1 or 1A, or a deuterated form, salt, solvate or hydrate thereof, wherein R^{1A} is represented by formula (A2),

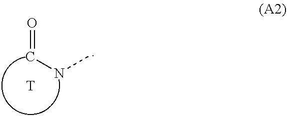

Formula (A2) includes formula (A1) when Z is ═O.
2A. The compound according to paragraph 1 or paragraph 1A, or a deuterated form, salt, solvate or hydrate thereof, wherein R^{1A} is represented by formula (A2′),

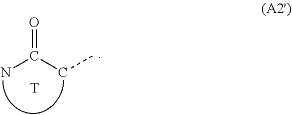

Formula (A2′) includes formula (A1′) when Z is ═O.
3. The compound according to any one of paragraphs 1, 1A, 2 or 2A or a deuterated form, salt, solvate or hydrate thereof, wherein the monocyclic 5- to 7-membered heterocycloalkyl group, the fused bicyclic 6- to 10-membered heterocycloalkyl group or the bridged bicyclic 6- to 9-membered heterocycloalkyl group, in addition to the intervening nitrogen atom (e.g. in formula (A1) or formula (A2)), each comprise 1, 2 or 3 heteroatoms, wherein each heteroatom is independently selected from an oxygen atom, a nitrogen atom and a sulphur.
   As each heterocycloalkyl group contains a nitrogen atom (as represented by T in formula (A1), (A1′), (A2) or (A2′)), the monocyclic heterocycloalkyl group, the fused bicyclic heterocycloalkyl group and the bridged bicyclic heterocycloalkyl group may each contain a total of 2, 3 or 4 heteroatoms, where at least one of the heteroatoms is a nitrogen atom and the other 1, 2 or 3 heteroatoms is each independently selected from an oxygen atom, a nitrogen atom and a sulphur atom.
4. The compound according to paragraph 3 or a deuterated form, salt, solvate or hydrate thereof, wherein the monocyclic 5- to 7-membered heterocycloalkyl group, the fused bicyclic 6- to 10-membered heterocycloalkyl group or the bridged bicyclic 6- to 9-membered heterocycloalkyl group, in addition to the intervening nitrogen atom (e.g. in formula (A1), (A1′), (A2) or (A2′)), each comprise 1 heteroatom, which is an oxygen atom, a nitrogen atom or a sulphur atom.
   The monocyclic heterocycloalkyl group, the fused bicyclic heterocycloalkyl group and the bridged bicyclic heterocycloalkyl group may each contain a total of 2 heteroatoms, which are both nitrogen atoms; one oxygen atom and one nitrogen atom; or one nitrogen atom and one sulphur atom.
5. The compound according to paragraph 3 or paragraph 4 or a deuterated form, salt, solvate or hydrate thereof, wherein the monocyclic 5- to 7-membered heterocycloalkyl group, the fused bicyclic 6- to 10-membered heterocycloalkyl group or the bridged bicyclic 6- to 9-membered heterocycloalkyl group, in addition to the intervening nitrogen atom (e.g. in formula (A1) or formula (A2)), each comprise 1 heteroatom, which is an oxygen atom or a nitrogen atom.
   The monocyclic heterocycloalkyl group, the fused bicyclic heterocycloalkyl group and the bridged bicyclic heterocycloalkyl group may each contain a total of 2 heteroatoms, which are both nitrogen atoms; or one oxygen atom and one nitrogen atom.
6. The compound according to any one of paragraphs 3 to 5 or a deuterated form, salt, solvate or hydrate thereof, wherein the monocyclic 5- to 7-membered heterocycloalkyl group, the fused bicyclic 6- to 10-membered heterocycloalkyl group or the bridged bicyclic 6- to 9-membered heterocycloalkyl group, in addition to the intervening nitrogen atom (e.g. in formula (A1), (A1′), (A2) or (A2′)), each comprise 1 heteroatom, which is a nitrogen atom.
   The monocyclic heterocycloalkyl group, the fused bicyclic heterocycloalkyl group and the bridged bicyclic heterocycloalkyl group may each contain a total of 2 heteroatoms, which are both nitrogen atoms.
7. The compound according to paragraph 1, 1A, 2 or 2A or a deuterated form, salt, solvate or hydrate thereof, wherein the monocyclic 5- to 7-membered heterocycloalkyl group, the fused bicyclic 6- to 10-membered heterocycloalkyl group or the bridged bicyclic 6- to 9-membered heterocycloalkyl group, each comprise only 1 heteroatom (e.g. the intervening nitrogen atom in formula (A1), (A1′), (A2) or (A2′)).
8. The compound according to any one of paragraphs 1, 1A, 2, 2A or 3 to 7 or a deuterated form, salt, solvate or hydrate thereof, wherein the monocyclic 5- to 7-membered heterocycloalkyl group is a monocyclic 5- or 6-membered heterocycloalkyl group optionally substituted with one or more R^{S1}.
9. The compound according to any one of paragraphs 1, 1A, 2, 2A or 3 to 8 or a deuterated form, salt, solvate or hydrate thereof, wherein the monocyclic 5- to 7-membered heterocycloalkyl group is a monocyclic 5-membered heterocycloalkyl group optionally substituted with one or more R^{S1}.
10. The compound according to any one of paragraphs 1, 1A, 2, 2A or 3 to 9 or a deuterated form, salt, solvate or hydrate thereof, wherein T is selected from a pyrrolidine-2-one ring, a 1,3-dihydro-2H-pyrrol-2-one ring, an imidazolidin-2-one ring, a 1,3-dihydro-2H-imidazol-2-one ring, an oxazol-2(3H)-one ring, an oxazolidin-2-one ring a thiazol-2(3H)-one ring and a thiazolidin-2-one ring. Each ring is optionally substituted with one or more R^{S1}. These rings are all 5-membered monocyclic rings.
11. The compound according to paragraph 10 or a deuterated form, salt, solvate or hydrate thereof, wherein T is selected from a pyrrolidin-2-one ring, an imidazolidin-2-one ring and a 1,3-dihydro-2H-imidazol-2-one ring.
12. The compound according to any one of paragraphs 1, 1A, 2, 2A or 3 to 8 or a deuterated form, salt, solvate or hydrate thereof, wherein the monocyclic 5- to 7-membered heterocycloalkyl group is a monocyclic 6-membered heterocycloalkyl group optionally substituted with one or more R^{S1}.
13. The compound according to any one of paragraphs 1, 1A, 2, 2A or 3 to 8 or paragraph 12 or a deuterated form, salt, solvate or hydrate thereof, wherein T is selected from a piperidin-2-one ring, a tetrahydropyrimidin-2(1H)-one ring, a pyridazin-3(2H)-one ring, a pyrimidin-4(3H)-one ring, a pyrazin-2(1H)-one ring, a pyridin-2(1H)-one ring, a tetrahydropyrimidin-2(1H)-one ring and a 1,3-oxazinan-2-one ring. Each ring is optionally substituted with one or more R^{S1}. These rings are all 6-membered monocyclic rings.
14. The compound according to any one of paragraphs 1, 1A, 2, 2A or 3 to 8 or a deuterated form, salt, solvate or hydrate thereof, wherein the monocyclic 5- to 7-membered heterocycloalkyl group is a monocyclic 7-membered heterocycloalkyl group optionally substituted with one or more R^{S1}.
15. The compound according to any one of paragraphs 1, 1A, 2, 2A or 3 to 8 or paragraph 14 or a deuterated form, salt, solvate or hydrate thereof, wherein T is an azepan-2-one ring. The ring is optionally substituted with one or more R^{S1}. This ring is a 7-membered monocyclic ring.
16. The compound according to any one of paragraphs 1, 1A, 2, 2A or 3 to 15 or a deuterated form, salt, solvate or hydrate thereof, wherein the monocyclic 5- to 7-membered heterocycloalkyl group is unsubstituted.
17. The compound according to any one of paragraphs 1, 1A, 2, 2A or 3 to 15 or a deuterated form, salt, solvate or hydrate thereof, wherein the monocyclic 5- to 7-membered heterocycloalkyl group is substituted with one, two or three R^{S1}.
18. The compound according to paragraph 17 or a deuterated form, salt, solvate or hydrate thereof, wherein the monocyclic 5- to 7-membered heterocycloalkyl group is substituted with one or two R^{S1}.
19. The compound according to paragraph 18 or a deuterated form, salt, solvate or hydrate thereof, wherein the monocyclic 5- to 7-membered heterocycloalkyl group is substituted with one R^{S1}.
20. The compound according to any one of paragraphs 1, 1A, 2, 2A or 3 to 7 or a deuterated form, salt, solvate or hydrate thereof, wherein the fused bicyclic 6- to 10-membered heterocycloalkyl group has a first 5- or 6-membered ring including the intervening carbon and nitrogen atoms (e.g. in formula (A1), (A1′), (A2) or (A2′) above) and a second ring fused to the first 5- or 6-membered ring. Each of the first 5- or 6-membered ring and the second ring is optionally substituted with one or more R^{S1}.
21. The compound according to paragraph 20 or a deuterated form, salt, solvate or hydrate thereof, wherein the first 5- or 6-membered ring and the second ring are ortho-fused (e.g. there are 2 atoms (i.e. fusion atoms) that are common to both the first 5- or 6-membered ring and the second ring).
22. The compound according to paragraph 20 or paragraph 21 or a deuterated form, salt, solvate or hydrate thereof, wherein the second ring is a 3- to 6-membered ring optionally substituted with one or more R^{S1}. The total number of ring atoms (e.g. fusion atoms and peripheral atoms) in the first 5- or 6-membered ring and the second ring is 6 to 10 atoms including the intervening carbon and nitrogen atoms (e.g. in formula (A1), (A1′), (A2) or (A2′)).
23. The compound according to paragraph 22 or a deuterated form, salt, solvate or hydrate thereof, wherein the second ring is a 3-, 5- or 6-membered ring optionally substituted with one or more R^{S1}.
24 The compound according to paragraph 23 or a deuterated form, salt, solvate or hydrate thereof, wherein the second ring is a 3-membered ring optionally substituted with one or more R^{S1}. Typically, the 3-membered ring is a saturated 3-membered ring.
25. The compound according to any one of paragraphs 1, 1A, 2, 2A or 3 to 7 or paragraphs 20 to 24 or a deuterated form, salt, solvate or hydrate thereof, wherein T is a 3-azabicyclo[4.1.0]heptan-2-one group optionally substituted with one or more R^{S1}. This group has a first 6-membered ring fused to a second 3-membered ring.
26. The compound according to paragraph 23 or a deuterated form, salt, solvate or hydrate thereof, wherein the second ring is a 6-membered ring optionally substituted with one or more R^{S1}.
27. The compound according to paragraph 25 or a deuterated form, salt, solvate or hydrate thereof, wherein the 6-membered ring is aromatic.
28. The compound according to paragraph 25 or a deuterated form, salt, solvate or hydrate thereof, wherein the 6-membered ring is saturated.
29. The compound according to any one of paragraphs 1, 1A, 2, 2A,r 3 to 7, 20 to 23 or 26 to 28 or a deuterated form, salt, solvate or hydrate thereof, wherein T is selected from a quinolin-2(1H)-one group, an isoquinolin-1(2H)-one group, a 1,4-dihydroisoquinolin-3(2H)-one group, a 3,4-dihydro-2H-1\2-quinazolin-2-one group 1,3-dihydro-2H benzo[d]imidazol-2-one group. Each group is optionally substituted with one or more R^{S1}. These groups comprise a second ring that is a 6-membered ring.
30. The compound according to any one of paragraphs 20 to 29 or a deuterated form, salt, solvate or hydrate thereof, wherein the first 5- or 6-membered ring including the intervening carbon and nitrogen atoms is a 6-membered ring including the intervening carbon and nitrogen atoms (e.g. in formula (A1), (A1′), (A2) or (A2′)) optionally substituted with one or more R^{S1}.
31. The compound according to any one of paragraphs 1, 1A, 2, 2A, 3 to 7 or 20 to 30 or a deuterated form, salt, solvate or hydrate thereof, wherein the fused bicyclic 6- to 10-membered heterocycloalkyl group is unsubstituted.
32. The compound according to any one of paragraphs 1, 1A, 2, 2A, 3 to 7 or 20 to 30 or a deuterated form, salt, solvate or hydrate thereof, wherein the fused bicyclic 6- to 10-membered heterocycloalkyl group is substituted with one, two or three R^{S1}.
33 The compound according to paragraph 32 or a deuterated form, salt, solvate or hydrate thereof, wherein the fused bicyclic 6- to 10-membered heterocycloalkyl group is substituted with one or two R^{S1}.
34. The compound according to paragraph 33 or a deuterated form, salt, solvate or hydrate thereof, wherein the fused bicyclic 6- to 10-membered heterocycloalkyl group is substituted with one R^{S1}.
35. The compound according to any one of paragraphs 1, 1A, 2, 2A or 3 to 7 or a deuterated form, salt, solvate or hydrate thereof, wherein the bridged bicyclic 6- to 9-membered heterocycloalkyl group has a 5- to 7-membered ring including the intervening carbon and nitrogen atoms (e.g. in formula (A1), (A1′), (A2) or (A2′)) and a bridge having 1 or 2 atoms, wherein each of the 5- to 7-membered ring and the bridge is optionally substituted with one or more R^{S1}. The bridge is typically between non-adjacent atoms of the 5- to 7-membered ring.
36. The compound according to paragraph 35 or a deuterated form, salt, solvate or hydrate thereof, wherein the 5- to 7-membered ring including the intervening carbon and nitrogen atoms (e.g. in formula (A1), (A1′), (A2) or (A2′)) is a 6-membered ring optionally substituted with one or more R^{S1}.
37 The compound according to paragraph 35 or paragraph 36 or a deuterated form, salt, solvate or hydrate thereof, wherein the bridge having 1 or 2 atoms between non-adjacent atoms of the 5- to 7-membered ring is a bridge having 1 atom optionally substituted with one or more R^{S1}.
38. The compound according to any one of paragraphs 1, 1A, 2, 2A, 3 to 7 or 35 to 37 or a deuterated form, salt, solvate or hydrate thereof, wherein T is a 2-azabicyclo[2.2.1]heptan-3-one group optionally substituted with one or more R^{S1}.
39. The compound according to any one of paragraphs 1, 1A, 2, 2A, 3 to 7 or 35 to 38 or a deuterated form, salt, solvate or hydrate thereof, wherein the bridged bicyclic 6- to 9-membered heterocycloalkyl group is unsubstituted.
40. The compound according to any one of paragraphs 1, 1A, 2, 2A, 3 to 7 or 35 to 38 or a deuterated form, salt, solvate or hydrate thereof, wherein the bridged bicyclic 6- to 9-membered heterocycloalkyl group is substituted with one, two or three R^{S1}.
41. The compound according to paragraph 40 or a deuterated form, salt, solvate or hydrate thereof, wherein the bridged bicyclic 6- to 9-membered heterocycloalkyl group is substituted with one or two R^{S1}.
42. The compound according to paragraph 41 or a deuterated form, salt, solvate or hydrate thereof, wherein the bridged bicyclic 6- to 9-membered heterocycloalkyl group is substituted with one R^{S1}.
43. The compound according to any one of paragraphs 1, 1A, 2, 2A, 3 to 15, 17 to 30 or 32 to 42 or a deuterated form, salt, solvate or hydrate thereof, wherein each R^{S2} is selected from hydroxy, chloro, bromo, fluoro, CN and nitro.
44. The compound according to paragraph 42 or a deuterated form, salt, solvate or hydrate thereof, wherein each R^{S2} is selected from hydroxy, fluoro, CN and nitro.
45. The compound according to paragraph 42 or a deuterated form, salt, solvate or hydrate thereof, wherein each R^{S2} is selected from hydroxy, chloro, bromo and fluoro.
46. The compound according to any one of paragraphs 43 to 45 or a deuterated form, salt, solvate or hydrate thereof, wherein each R^{S2} is selected from hydroxy and fluoro.
47. The compound according to paragraph 46 or a deuterated form, salt, solvate or hydrate thereof, wherein each R^{S2} is fluoro.
48. The compound according to any one of paragraphs 1, 1A, 2, 2A, 3 to 15, 17 to 30 or 32 to 42 or a deuterated form, salt, solvate or hydrate thereof, wherein each R^{S1} is selected from C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.
49. The compound according to paragraph 48 or a deuterated form, salt, solvate or hydrate thereof, wherein each R^{S1} is selected from C₁₋₆alkyl, C₁₋₆alkoxy, C₃₋₁₂cycloalkyl, hydroxy, chloro, bromo, fluoro and CN, wherein the C₁₋₆alkyl and the C₃₋₁₂cycloalkyl is each optionally substituted with one or more R^{S2}.
50. The compound according to paragraph 49 or a deuterated form, salt, solvate or hydrate thereof, wherein each R^{S1} is selected from C₁₋₆alkyl, C₁₋₆alkoxy, C₃₋₁₂cycloalkyl, hydroxy and chloro, wherein the C₁₋₆alkyl and the C₃₋₁₂cycloalkyl is each optionally substituted with one or more R^{S2}.
51. The compound according to paragraph 50 or a deuterated form, salt, solvate or hydrate thereof, wherein each R^{S1} is selected from unsubstituted C₁₋₆alkyl, halo-C₁₋₆alkyl (e.g. unsubstituted halo-C₁₋₆alkyl), fluoro and chloro.
52. The compound according to paragraph 51 or a deuterated form, salt, solvate or hydrate thereof, wherein each R^{S1} is selected from unsubstituted C₁₋₆alkyl, fluoro-C₁₋₆alkyl (e.g. unsubstituted fluoro-C₁₋₆alkyl), fluoro and chloro.
53 The compound according to paragraph 52 or a deuterated form, salt, solvate or hydrate thereof, wherein each R^{S1} is selected from unsubstituted methyl, trifluoromethyl and chloro.
54. The compound according to any one of paragraphs 1, 1A, 2, 2A, 3 to 8, 12, 13, 16 to 19 or 43 to 53 or a deuterated form, salt, solvate or hydrate thereof, wherein R^{1A} is represented by formula (B1),

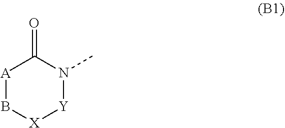

wherein either:
(a) X—Y is selected from CR^{3A}═CR^{2A}, CR^{3A}═N, N═CR^{2A}, N═N, C(R^{3A})₂—C(R^{2A})₂, C(R^{3A})₂—NR^{2A}, NR^{3A}—C(R^{2A})₂, NR^{3A}—NR^{2A}, C(R^{3A})₂—O, O—C(R^{2A})₂ C(R^{3A})₂—S and S—C(R^{2A})₂; and
   A-B is selected from CR^{5A}═CR^{4A}, CR^{5A}═N, N═CR^{4A}, N═N, C(R^{5A})₂—C(R^{4A})₂, C(R^{5A})₂—NR^{4A}, NR^{5A}—C(R^{4A})₂, NR^{5A}—NR^{4A}, C(R^{5A})₂—O, O—C(R^{4A})₂, C(R^{5A})₂—S and S—C(R^{4A})₂; or
(b) A is selected from C(R^{5A})₂, NR^{5A}, O and S;
   Y is selected from C(R^{2A})₂ and NR^{2A}; and
   B—X is selected from CR^{4A}═CR^{3A}, CR^{4A}═N and N═CR^{3A};
   wherein each R^{2A}, R^{3A}, R^{4A} and R^{5A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
   each R^{S2} is selected from hydroxy, halo, CN and nitro.

Formula (B1) is a monocyclic 6-membered heterocycloalkyl group. R^{S1} as defined above is represented by each of R^{2A}, R^{3A}, R^{4A} and R^{5A}.

Typically, when any of R^{2A}, R^{3A}, R^{4A} and R^{5A} is directly bonded to a nitrogen atom, then the R^{2A}, R^{3A}, R^{4A} and R^{5A} directly bonded to a nitrogen atom may be independently selected from hydrogen, C₁₋₆alkyl, C₂₋₆alkenyl, C₂₋₆alkynyl, and C₃₋₁₂cycloalkyl, wherein the C₁₋₆alkyl, the C₂₋₆alkenyl, the C₂₋₆alkynyl and the C₃₋₁₂cycloalkyl is each optionally substituted with one or more R^{S2}. In general, it is preferred that the substituent at a nitrogen atom is either hydrogen, C₁₋₆alkyl, C₂₋₆alkenyl, C₂₋₆alkynyl or C₃₋₁₂cycloalkyl.
55. The compound according to paragraph 54 or a deuterated form, salt, solvate, or hydrate thereof, wherein X—Y is selected from CR^{3A}—CR^{2A}, CR^{3A}═N, N═CR^{2A}, C(R^{3A})₂—C(R^{2A})₂, C(R^{3A})₂—NR^{2A}, NR^{3A}—C(R^{2A})₂ and O—C(R^{2A})₂.
56. The compound according to paragraph 54 or 55 or a deuterated form, salt, solvate, or hydrate thereof, wherein X—Y is selected from CR^{3A}═CR^{2A}, CR^{3A}═N, N═CR^{2A} and C(R^{3A})₂—C(R^{2A})₂.
57. The compound according to any one of paragraphs 54 to 56 or a deuterated form, salt, solvate, or hydrate thereof, wherein A-B is selected from CR^{5A}═CR^{4A}, CR^{5A}═N, N═CR^{4A}, C(R^{5A})₂—C(R^{4A})₂, C(R^{5A})₂—NR^{4A}, NR^{5A}—C(R^{4A})₂ and O—C(R^{2A})₂.
58 The compound according to paragraph 57 or a deuterated form, salt, solvate, or hydrate thereof, wherein A-B is selected from CR^{5A}═CR^{4A}, CR^{5A}═N, N═CR^{4A}, C(R^{5A})₂—C(R^{4A})₂, NR^{5A}—C(R^{4A})₂ and O—C(R^{2A})₂.
59. The compound according to paragraph 58 or a deuterated form, salt, solvate, or hydrate thereof, wherein A-B is selected from CR^{5A}═CR^{4A}, CR^{5A}_N, C(R^{5A})₂—C(R^{4A})₂, and NR^{5A}—C(R^{4A})₂.
60. The compound according to paragraph 59 or a deuterated form, salt, solvate, or hydrate thereof, wherein A-B is C(R^{5A})₂—C(R^{4A})₂.
61. The compound according to any one of paragraphs 54 to 60 or a deuterated form, salt, solvate, or hydrate thereof, wherein when A is C(R^{5A})₂, then Y is C(R^{2A})₂ and B—X is selected from CR^{4A}═CR^{3A}, CR^{4A}═N and N═CR^{3A}; or when A is NR^{5A}; then Y is C(R^{2A})₂ and B—X is selected from CR^{4A}═CR^{3A} and CR^{4A}═N.
62. The compound according to any one of paragraphs 54 to 61 or a deuterated form, salt, solvate, or hydrate thereof, wherein R^{1A} is represented by one of the following formulae,

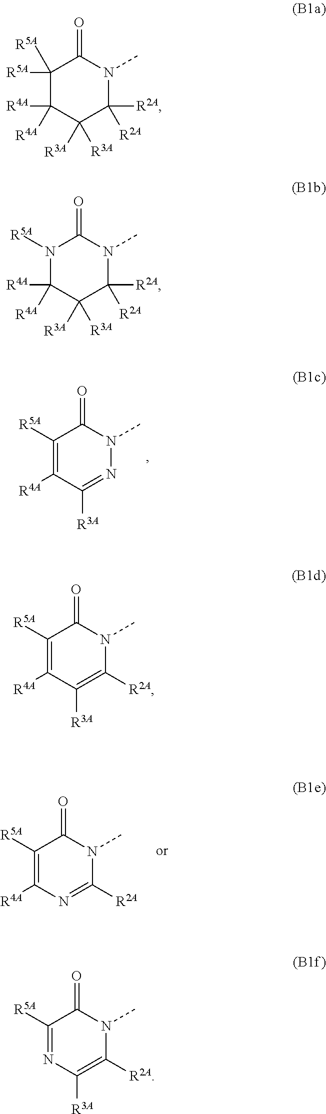

63. The compound according to any one of paragraphs 54 to 62 or a deuterated form, salt, solvate, or hydrate thereof, wherein when there is two R^{2A} (e.g. in formula (B1) when Y is C(R^{2A})₂ or when X—Y is selected from C(R^{3A})₂—C(R^{2A})₂ and NR^{3A}—C(R^{2A})₂; in formula (B1a) or in formula (B1b)), two R^{3A} (e.g. in formula (B1) when X—Y is selected from C(R^{3A})₂—C(R^{2A})₂ and C(R^{3A})₂—NR^{2A}; in formula (B1a) or in formula (B1b)), two R^{4A} (e.g. in formula (B1) when A-B is selected from C(R^{5A})₂—C(R^{4A})₂ and NR^{5A}—C(R^{4A})₂; in formula (B1a) or in formula (B1b)); or two R^{5A} (e.g. in formula (B1) when A-B is selected from C(R^{5A})₂—C(R⁴⁴)₂ and C(R⁵⁴)₂—NR^{4A} or when A is C(R^{5A})₂; in formula (B1a) or in formula (B1b)), then at least one of the two R^{2A}, R^{3A}, R^{4A} or R^{5A} is hydrogen.

Typically, the other of the two R^{2A}, R^{3A}, R^{4A} or R^{5A} is selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}.
64. The compound according to any one of paragraphs 54 to 63 or a deuterated form, salt, solvate, or hydrate thereof, wherein R^{1A} is represented by one of the following formulae,

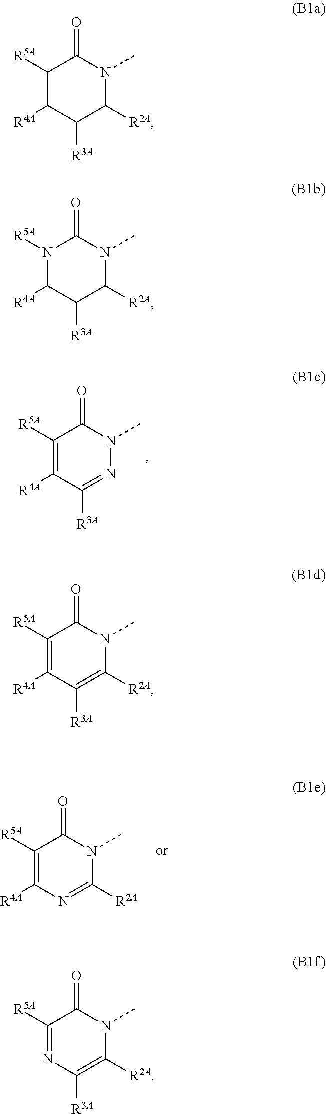

65. The compound according to any one of paragraphs 54 to 64 or a deuterated form, salt, solvate, or hydrate thereof, wherein each R^{2A}, R^{3A}, R^{4A} and R^{5A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.
66. The compound according to paragraph 65 or a deuterated form, salt, solvate or hydrate thereof, wherein each R^{2A}, R^{3A}, R^{4A} and R^{5A} is the same or different and is independently selected from hydrogen, C₁₋₆alkyl, C₁₋₆alkoxy, C₃₋₁₂cycloalkyl, hydroxy, chloro, bromo, fluoro and CN, wherein the C₁₋₆alkyl and the C₃₋₁₂cycloalkyl is each optionally substituted with one or more R^{S2}.
67. The compound according to paragraph 66 or a deuterated form, salt, solvate or hydrate thereof, wherein each R^{2A}, R^{3A}, R^{4A} and R^{5A} is the same or different and is independently selected from hydrogen, C₁₋₆alkyl, C₁₋₆alkoxy, C₃₋₁₂cycloalkyl, hydroxy and chloro, wherein the C₁₋₆alkyl and the C₃₋₁₂cycloalkyl is each optionally substituted with one or more R^{S2}.
68. The compound according to paragraph 67 or a deuterated form, salt, solvate or hydrate thereof, wherein each R^{2A}, R^{3A}, R^{4A} and R^{5A} is the same or different and is independently selected from hydrogen, unsubstituted C₁₋₆alkyl, halo-C₁₋₆alkyl (unsubstituted halo-C₁₋₆alkyl) and chloro.
69. The compound according to paragraph 68 or a deuterated form, salt, solvate or hydrate thereof, wherein each R^{2A}, R^{3A}, R^{4A} and R^{5A} is the same or different and is independently selected from hydrogen, unsubstituted C₁₋₆alkyl, fluoro-C₁₋₆alkyl (unsubstituted fluoro-C₁₋₆alkyl) and chloro.
70. The compound according to paragraph 69 or a deuterated form, salt, solvate or hydrate thereof, wherein each R^{2A}, R^{3A}, R^{4A} and R^{5A} is the same or different and is independently selected from hydrogen, unsubstituted methyl, trifluoromethyl and chloro.
71. The compound according to any one of paragraphs 1, 1A, 2, 2A, 3 to 8, 12, 13, 16 to 19 or 43 to 53 or a deuterated form, salt, solvate or hydrate thereof, wherein R^{1A} is represented by formula (B1′),

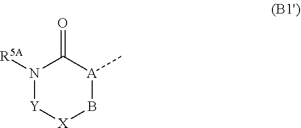

wherein either:
(a) Y—X is selected from CR^{4A}═CR^{3A}, CR^{4A}═N, N═CR^{3A}, N═N, C(R^{4A})₂—C(R^{3A})₂, C(R^{4A})₂—NR^{3A}, NR^{4A}—C(R^{3A})₂, NR^{4A}—NR^{3A}, C(R^{4A})₂—O, O—C(R^{3A})₂ C(R^{4A})₂—S and S—C(R^{3A})₂; and
   B-A is selected from C(R²)₂—CR^{6A}, NR^{2A}—CR^{6A}, O—CR^{6A} and S—CR^{6A}; or
(b) Y is selected from C(R^{4A})₂ and NR^{4A};
   X—B is selected from CR^{3A}═CR^{2A}, CR^{3A}═N and N═CR^{2A}; and
   A is CR^{6A};
   wherein each R^{2A}, R^{3A} and R^{4A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2};
   each R^{5A} and R^{6A} is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
   each R^{S2} is selected from hydroxy, halo, CN and nitro.

Formula (B1′) is a monocyclic 6-membered heterocycloalkyl group. R^{S1} as defined above is represented by each of R^{2A}, R^{3A}, R^{4A}, R^{5A} and R^{6A}.
72. The compound according to paragraph 71 or a deuterated form, salt, solvate, or hydrate thereof, wherein Y—X is selected from CR^{4A}═CR^{3A}, CR^{4A}═N, N═CR^{3A}, C(R^{4A})₂—C(R^{3A})₂, C(R^{4A})₂—NR^{3A}, NR^{4A}—C(R^{3A})₂ and O—C(R^{3A})₂.
73. The compound according to paragraph 71 or 72 or a deuterated form, salt, solvate, or hydrate thereof, wherein Y—X is selected from CR^{4A}═CR^{3A}, CR^{4A}═N, N═CR^{3A} and C(R^{4A})₂—C(R^{3A})₂.
74. The compound according to any one of paragraphs 71 to 73 or a deuterated form, salt, solvate, or hydrate thereof, wherein Y—X is selected from CR^{4A}═CR^{3A}, CR^{4A}═N and C(R^{4A})₂—C(R^{3A})₂.
75. The compound according to any one of paragraphs 71 to 74 or a deuterated form, salt, solvate, or hydrate thereof, wherein B-A is selected from C(R²)₂—CR^{6A}, NR^{2A}—CR^{6A} and O—CR^{6A}.
76. The compound according to paragraph 75 or a deuterated form, salt, solvate, or hydrate thereof, wherein B-A is selected from C(R^{2A})₂—CR^{6A} and NR^{2A}—CR^{6A}
77. The compound according to paragraph 76 or a deuterated form, salt, solvate, or hydrate thereof, wherein B-A is C(R^{2A})₂—CR^{6A}.
78. The compound according to any one of paragraphs 71 to 77 or a deuterated form, salt, solvate, or hydrate thereof, wherein R^{1A} is represented by one of the following formulae,

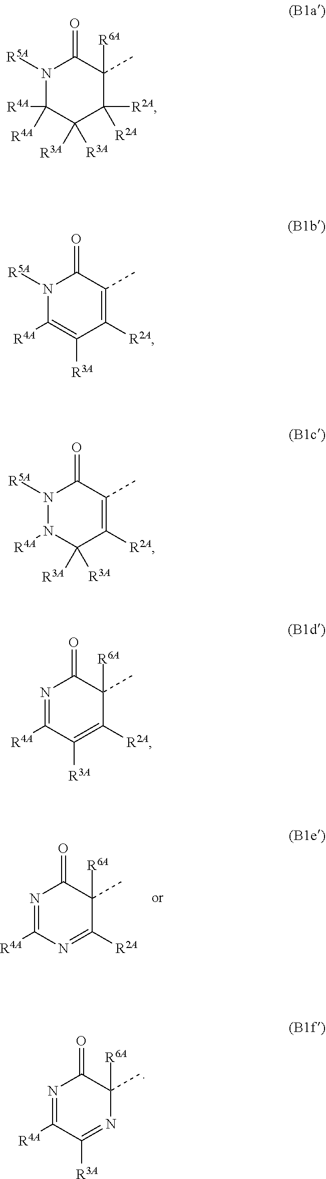

79. The compound according to any one of paragraphs 71 to 78 or a deuterated form, salt, solvate, or hydrate thereof, wherein when there is two R^{4A}, two R^{3A} or two R^{2A}, then at least one of the two R^{4A}, R^{3A} and R^{2A} is hydrogen. Typically, the other of the two R^{4A}, R^{3A} or R^{2A} is selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}.
80. The compound according to any one of paragraphs 71 to 79 or a deuterated form, salt, solvate, or hydrate thereof, wherein R^{6A} is hydrogen or C₁₋₆alkyl, preferably hydrogen.
81. The compound according to any one of paragraphs 71 to 80 or a deuterated form, salt, solvate, or hydrate thereof, wherein R^{5A} is selected from hydrogen, C₁₋₆alkyl, C₂₋₆alkenyl, C₂₋₆alkynyl and C₃₋₁₂cycloalkyl, wherein the C₁₋₆alkyl, the C₂₋₆alkenyl, the C₂₋₆alkynyl and the C₃₋₁₂cycloalkyl is each optionally substituted with one or more R^{S2}.
82. The compound according to paragraph 81 or a deuterated form, salt, solvate, or hydrate thereof, wherein R^{5A} is selected from hydrogen and C₁₋₆alkyl, preferably R^{5A} is hydrogen.
83. The compound according to any one of paragraphs 71 to 82 or a deuterated form, salt, solvate, or hydrate thereof, wherein R^{1A} is represented by one of the following formulae,

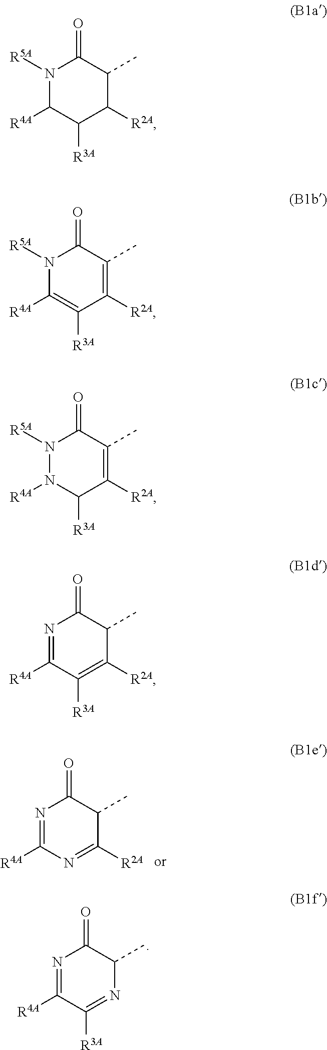

84. The compound according to any one of paragraphs 71 to 83 or a deuterated form, salt, solvate, or hydrate thereof, wherein each R^{2A}, R^{3A} and R^{4A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.
85. The compound according to paragraph 84 or a deuterated form, salt, solvate or hydrate thereof, wherein each R^{2A}, R^{3A} and R^{4A} is the same or different and is independently selected from hydrogen, C₁₋₆alkyl, C₁₋₆alkoxy, C₃₋₁₂cycloalkyl, hydroxy, chloro, bromo, fluoro and CN, wherein the C₁₋₆alkyl and the C₃₋₁₂cycloalkyl is each optionally substituted with one or more R^{S2}.
86. The compound according to paragraph 85 or a deuterated form, salt, solvate or hydrate thereof, wherein each R^{2A}, R^{3A} and R^{4A} is the same or different and is independently selected from hydrogen, C₁₋₆alkyl, C₁₋₆alkoxy, C₃₋₁₂cycloalkyl, hydroxy and chloro, wherein the C₁₋₆alkyl and the C₃₋₁₂cycloalkyl is each optionally substituted with one or more R^{S2}.
87. The compound according to paragraph 86 or a deuterated form, salt, solvate or hydrate thereof, wherein each R^{2A}, R^{3A} and R^{4A} is the same or different and is independently selected from hydrogen, unsubstituted C₁₋₆alkyl, halo-C₁₋₆alkyl (unsubstituted halo-C₁₋₆alkyl) and chloro.
88. The compound according to paragraph 87 or a deuterated form, salt, solvate or hydrate thereof, wherein each R^{2A}, R^{3A} and R^{4A} is the same or different and is independently selected from hydrogen, unsubstituted C₁₋₆alkyl, fluoro-C₁₋₆alkyl (unsubstituted fluoro-C₁₋₆alkyl) and chloro.
89. The compound according to paragraph 88 or a deuterated form, salt, solvate or hydrate thereof, wherein each R^{2A}, R^{3A} and R^{4A} is the same or different and is independently selected from hydrogen, unsubstituted methyl, trifluoromethyl and chloro.
90. The compound according to any one of paragraphs 1, 1A, 2, 2A, 3 to 11, 16 to 19 or 43 to 53 or a deuterated form, salt, solvate or hydrate thereof, wherein R^{1A} is represented by formula (C1),

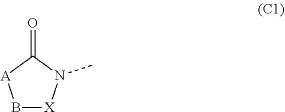

wherein either:
(a) X is selected from C(R^{2A})₂ and NR^{2A}; and
   A-B is selected from CR^{4A}═CR^{3A}, CR^{4A}═N, N═CR^{3A}, N═N, C(R^{4A})₂—C(R^{3A})₂, C(R^{4A})₂—NR^{3A}, NR^{4A}—C(R^{3A})₂, NR^{4A}—NR^{3A}, C(R^{4A})₂—O, O—C(R^{3A})₂, C(R⁴)₂—S and S—C(R^{3A})₂; or
(b) A is selected from C(R^{4A})₂, NR^{4A}, O and S; and
   B—X is selected from CR^{3A}═CR^{2A}, CR^{3A}═N, N═CR^{2A} and N═N;
   wherein each R^{2A}, R^{3A}, R^{4A} and R^{5A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
   each R^{S2} is selected from hydroxy, halo, CN and nitro.

Formula (C₁) is a monocyclic 5-membered heterocycloalkyl group. R^{S1} as defined above is represented by each of R^{2A}, R^{3A} and R^{4A}.
91. The compound according to paragraph 90 or a deuterated form, salt, solvate, or hydrate thereof, wherein A-B is selected from CR^{4A}═CR^{3A}, CR^{4A}═N, N═CR^{3A}, C(R^{4A})₂—C(R^{3A})₂, C(R^{4A})₂—NR^{3A}, NR^{4A}—C(R^{3A})₂ and NR^{4A}—NR^{3A}
92. The compound according to paragraph 90 or a deuterated form, salt, solvate, or hydrate thereof, wherein A-B is selected from CR^{4A}═CR^{3A}, N═CR^{3A}, C(R^{4A})₂—C(R^{3A})₂ and NR^{4A}—C(R^{3A})₂.
93. The compound according to paragraph 90 or a deuterated form, salt, solvate, or hydrate thereof, wherein A-B is C(R^{4A})₂—C(R^{3A})₂.
94. The compound according to any one of paragraphs 90 to 93, or a deuterated form, salt, solvate, or hydrate thereof, wherein X is C(R^{2A})₂.
95. The compound according to any one of paragraphs 90 to 94 or a deuterated form, salt, solvate, or hydrate thereof, wherein R^{1A} is represented by formula (C2),

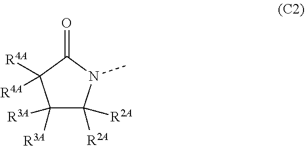

wherein each R^{2A}, R^{3A} and R^{4A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
each R^{S2} is selected from hydroxy, halo, CN and nitro.
96. The compound according to paragraph 95 or a deuterated form, salt, solvate, or hydrate thereof, wherein R^{1A} is represented by formula (C3),

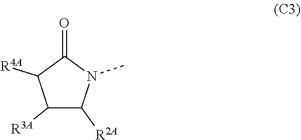

wherein R^{2A}, R^{3A} and R^{4A} is each independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
each R^{S2} is selected from hydroxy, halo, CN and nitro.
97. The compound according to any one of paragraphs 90 to 96 or a deuterated form, salt, solvate, or hydrate thereof, wherein each R^{2A}, R^{3A} and R^{4A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.
98. The compound according to paragraph 97 or a deuterated form, salt, solvate or hydrate thereof, wherein each R^{2A}, R^{3A} and R^{4A} is the same or different and is independently selected from hydrogen, C₁₋₆alkyl, C₁₋₆alkoxy, C₃₋₁₂cycloalkyl, hydroxy, chloro, bromo, fluoro and CN, wherein the C₁₋₆alkyl and the C₃₋₁₂cycloalkyl is each optionally substituted with one or more R^{S2}.
99. The compound according to paragraph 98 or a deuterated form, salt, solvate or hydrate thereof, wherein each R^{2A}, R^{3A} and R^{4A} is the same or different and is independently selected from hydrogen, C₁₋₆alkyl, C₁₋₆alkoxy, C₃₋₁₂cycloalkyl, hydroxy and chloro, wherein the C₁₋₆alkyl and the C₃₋₁₂cycloalkyl is each optionally substituted with one or more R^{S2}.
100. The compound according to paragraph 99 or a deuterated form, salt, solvate or hydrate thereof, wherein each R^{2A}, R^{3A} and R^{4A} is the same or different and is independently selected from hydrogen, unsubstituted C₁₋₆alkyl, halo-C₁₋₆alkyl (unsubstituted halo-C₁₋₆alkyl), fluoro and chloro.
101. The compound according to paragraph 100 or a deuterated form, salt, solvate or hydrate thereof, wherein each R^{2A}, R^{3A} and R^{4A} is the same or different and is independently selected from hydrogen, unsubstituted C₁₋₆alkyl, fluoro-C₁₋₆alkyl (unsubstituted fluoro-C₁₋₆alkyl), fluoro and chloro.
102. The compound according to paragraph 101 or a deuterated form, salt, solvate or hydrate thereof, wherein each R^{2A}, R^{3A} and R^{4A} is the same or different and is independently selected from hydrogen, unsubstituted methyl, trifluoromethyl and chloro.
103. The compound according to any one of paragraphs 1, 1A, 2, 2A, 3 to 11, 16 to 19 or 43 to 53 or a deuterated form, salt, solvate or hydrate thereof, wherein R^{1A} is represented by formula (C1′),

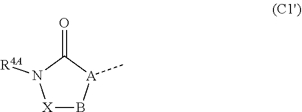

wherein either:
(b) X is selected from C(R^{3A})₂ and NR^{3A}; and
   B-A is selected from C(R^{2A})₂—CR^{5A}, C(R^{2A})₂—N, NR^{2A}—CR^{5A}, NR^{2A}—N, O—CR^{5A} and S—CR^{5A}₂; or
(b) X—B is selected from CR^{3A}═CR^{2A}, CR^{3A}═N, N═CR^{2A} and N═N;
   A is selected from CR^{5A} and N; and
   wherein each R^{2A}, R^{3A}, R^{4A} and R^{5A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
   each R^{S2} is selected from hydroxy, halo, CN and nitro.

Formula (C₁′) is a monocyclic 5-membered heterocycloalkyl group. R^{S1} as defined above is represented by each of R^{2A}, R^{3A} and R^{4A}.
104. The compound according to paragraph 103 or a deuterated form, salt, solvate, or hydrate thereof, wherein B-A is selected from C(R^{2A})₂—CR^{5A}, C(R^{2A})₂—N and NR^{2A}—CR^{5A}
105. The compound according to paragraph 103 or a deuterated form, salt, solvate, or hydrate thereof, wherein B-A is C(R^{2A})₂—CR^{5A}.
106. The compound according to any one of paragraphs 103 to 105, or a deuterated form, salt, solvate, or hydrate thereof, wherein X is C(R^{3A})₂.
107. The compound according to any one of paragraphs 103 to 105 or a deuterated form, salt, solvate, or hydrate thereof, wherein R^{1A} is represented by formula (C2′),

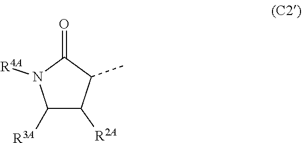

wherein each R^{2A}, R^{3A} and R^{4A} is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
each R^{S2} is selected from hydroxy, halo, CN and nitro.
108. The compound according to any one of paragraphs 103 to 107 or a deuterated form, salt, solvate, or hydrate thereof, wherein R^{5A} is hydrogen or C₁₋₆alkyl, preferably hydrogen.
109. The compound according to any one of paragraphs 103 to 108 or a deuterated form, salt, solvate, or hydrate thereof, wherein R^{4A} is selected from hydrogen, C₁₋₆alkyl, C₂₋₆alkenyl, C₂₋₆alkynyl and C₃₋₁₂cycloalkyl, wherein the C₁₋₆alkyl, the C₂₋₆alkenyl, the C₂₋₆alkynyl and the C₃₋₁₂cycloalkyl is each optionally substituted with one or more R^{S2}.
110. The compound according to any one of paragraphs 103 to 109 or a deuterated form, salt, solvate, or hydrate thereof, wherein each R^{2A} and R^{3A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.
111. The compound according to paragraph 110 or a deuterated form, salt, solvate or hydrate thereof, wherein each R^{2A} and R^{3A} is the same or different and is independently selected from hydrogen, C₁₋₆alkyl, C₁₋₆alkoxy, C₃₋₁₂cycloalkyl, hydroxy, chloro, bromo, fluoro and CN, wherein the C₁₋₆alkyl and the C₃₋₁₂cycloalkyl is each optionally substituted with one or more R^{S2}.
112. The compound according to paragraph 111 or a deuterated form, salt, solvate or hydrate thereof, wherein each R^{2A} and R^{3A} is the same or different and is independently selected from hydrogen, C₁₋₆alkyl, C₁₋₆alkoxy, C₃₋₁₂cycloalkyl, hydroxy and chloro, wherein the C₁₋₆alkyl and the C₃₋₁₂cycloalkyl is each optionally substituted with one or more R^{S2}.
113. The compound according to paragraph 112 or a deuterated form, salt, solvate or hydrate thereof, wherein each R^{2A} and R^{3A} is the same or different and is independently selected from hydrogen, unsubstituted C₁₋₆alkyl, halo-C₁₋₆alkyl (unsubstituted halo-C₁₋₆alkyl), fluoro and chloro.
114. The compound according to paragraph 113 or a deuterated form, salt, solvate or hydrate thereof, wherein each R^{2A} and R^{3A} is the same or different and is independently selected from hydrogen, unsubstituted C₁₋₆alkyl, fluoro-C₁₋₆alkyl (unsubstituted fluoro-C₁₋₆alkyl), fluoro and chloro.
115. The compound according to paragraph 114 or a deuterated form, salt, solvate or hydrate thereof, wherein each R^{2A} and R^{3A} is the same or different and is independently selected from hydrogen, unsubstituted methyl, trifluoromethyl and chloro.
116. The compound according to any one of paragraphs 1 to 115 or a deuterated form, salt, solvate, or hydrate thereof, wherein the compound is of formula (1-a),

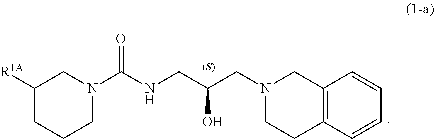

117. The compound according to any one of paragraphs 1 to 115 or a deuterated form, salt, solvate, or hydrate thereof, wherein the compound is of formula (1-b),

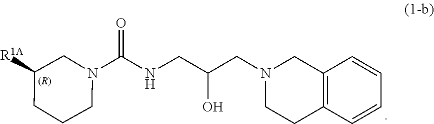

118. The compound according to any one of paragraphs 1 to 115 or a deuterated form, salt, solvate, or hydrate thereof, wherein the compound is of formula (1-c),

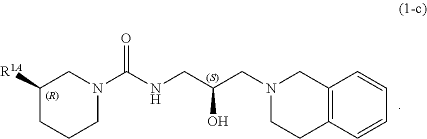

119. The compound according to any one of paragraphs 1 to 115, wherein the compound is of formula (1-1) or a salt, solvate or hydrate thereof,

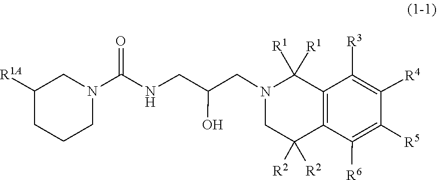

wherein each of R¹, R², R³, R⁴, R⁵ and R⁶ is the same or different and is independently selected from hydrogen and deuterium.

Formula (1-1) includes a “deuterated form” of the compound of formula (1). It is preferred that each R¹ is the same and/or each R² is the same.
120. The compound according to paragraph 119, wherein the compound is of formula (1-2) or a salt, solvate or hydrate thereof,

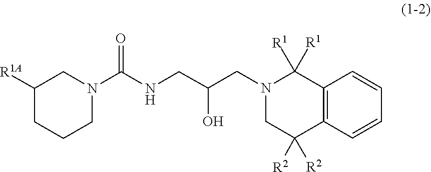

wherein each of R¹ and R² is the same or different and is independently selected from hydrogen and deuterium.

Formula (1-2) includes a “deuterated form” of the compound of formula (1). In Formula (1-2), R³, R⁴, R⁵ and R⁶ in formula (1-1) are hydrogen. It is preferred that each R¹ is the same and/or each R² is the same.
121. The compound according to paragraph 119 or paragraph 120, wherein the compound is of formula (1-1a) or a salt, solvate or hydrate thereof, or the compound is of formula (1-2a) or a salt, solvate or hydrate thereof,

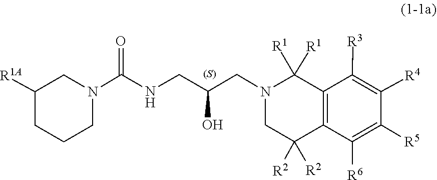

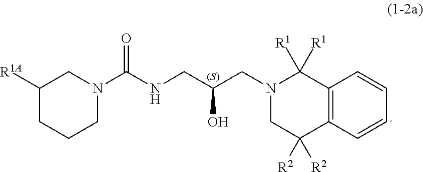

122. The compound according to paragraph 119 or paragraph 120, wherein the compound is of formula (1-1b) or a salt, solvate or hydrate thereof, or the compound is of formula (1-2b) or a salt, solvate or hydrate thereof,

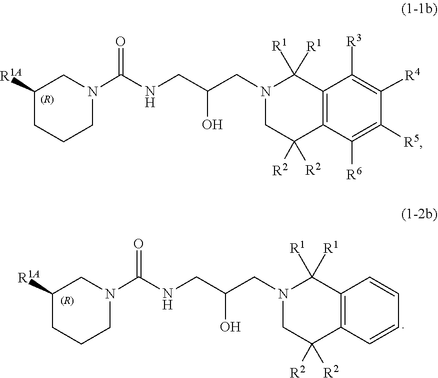

123. The compound according to paragraph 119 or paragraph 120, wherein the compound is of formula (1-1c) or a salt, solvate or hydrate thereof, or the compound is of formula (1-2c) or a salt, solvate or hydrate thereof,

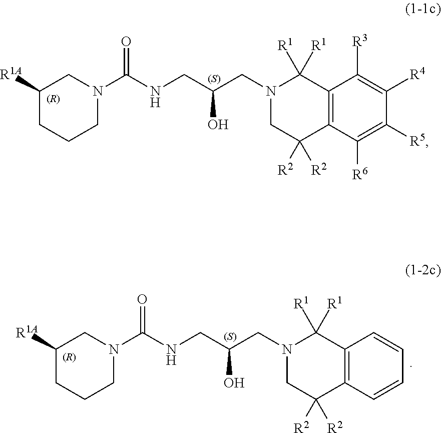

124. The compound according to any one of paragraphs 1 to 3 selected from:

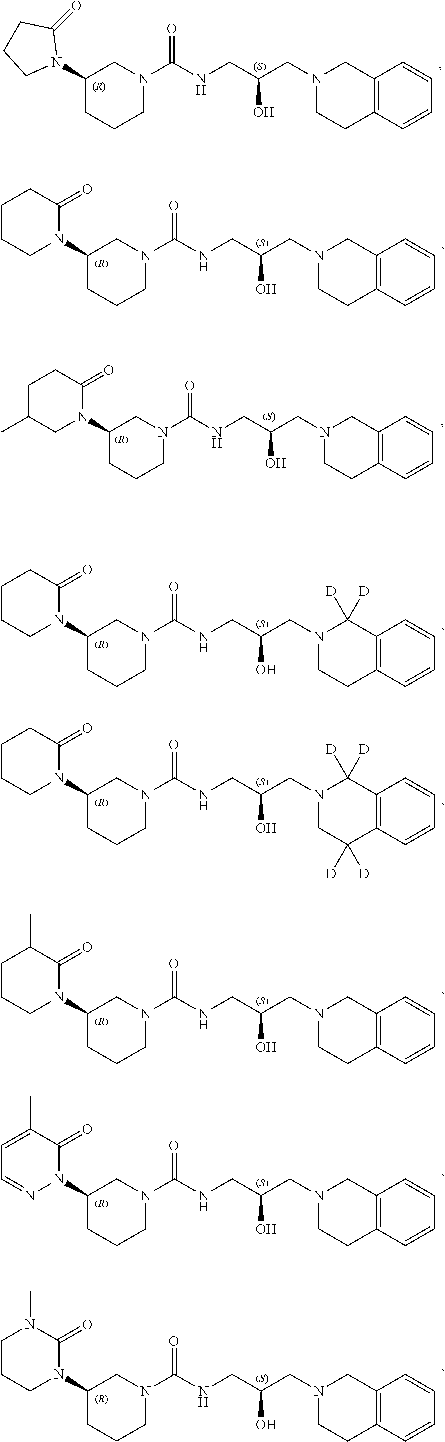

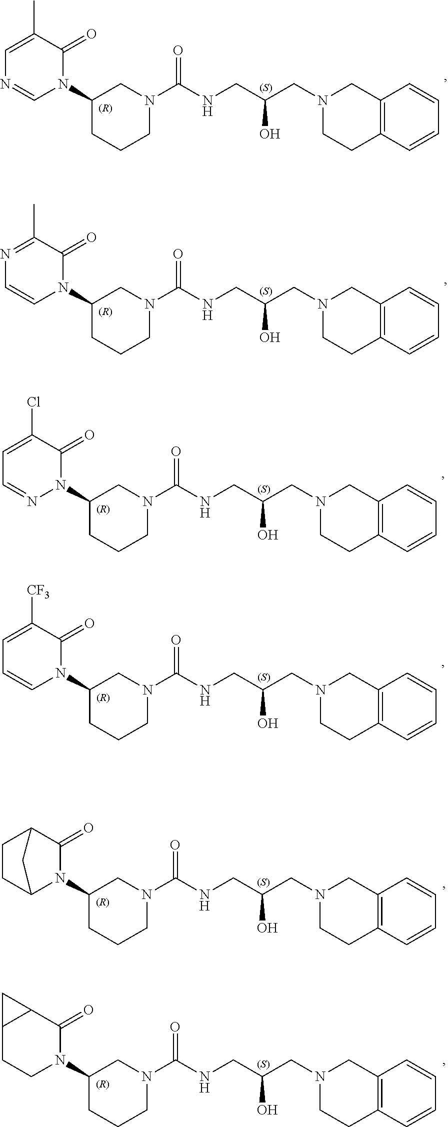

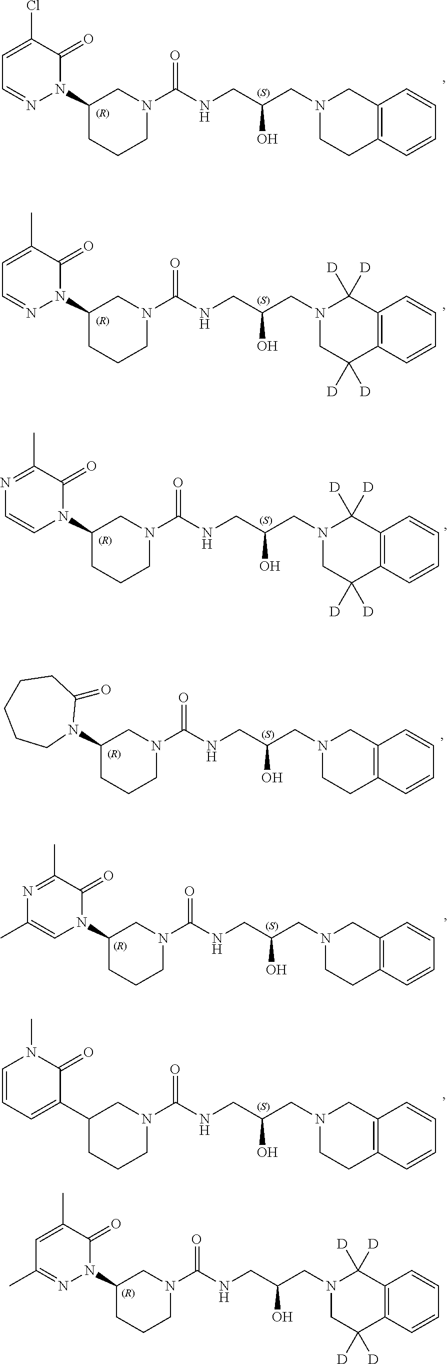

and a salt, solvate, or hydrate thereof.
125. The compound according to paragraph 124 selected from:

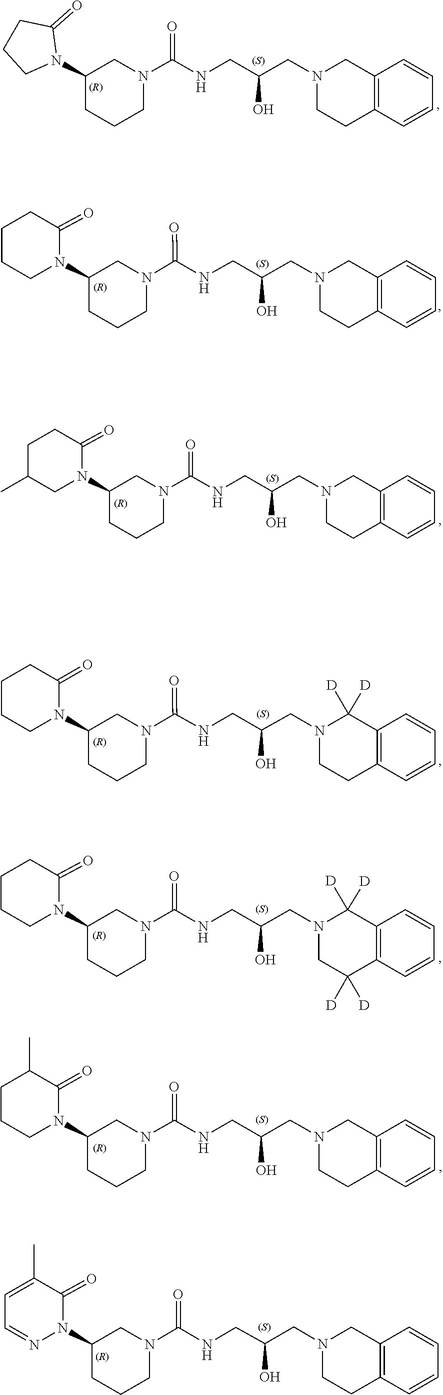

and a salt, solvate, or hydrate thereof.
126. A pharmaceutical composition, which comprises a compound according to any one of paragraphs 1 to 125, or a deuterated form, pharmaceutically acceptable salt, hydrate or solvate thereof. The pharmaceutical composition may further comprise a pharmaceutically acceptable excipient.
127. A compound according to any one of paragraphs 1 to 125, or a deuterated form, a pharmaceutically acceptable salt, hydrate or solvate thereof, or a pharmaceutical composition according to paragraph 126, for use in therapy and/or for use as a medicament.
128. A compound according to any one of paragraphs 1 to 125, or a deuterated form, a pharmaceutically acceptable salt, hydrate or solvate thereof, or a pharmaceutical composition according to paragraph 126, for use in the treatment of a proliferative disorder.
129. A compound according to any one of paragraphs 1 to 125, or a deuterated form, pharmaceutically acceptable salt, hydrate or solvate thereof, or a pharmaceutical composition according to paragraph 126, for use in the treatment cancer.
130. A compound or a pharmaceutical composition for use according to paragraph 129, wherein the cancer is selected from colorectal, ovarian, prostate, lung, breast, lymphoma/leukaemia, oesophageal, gastric, hepatocellular and brain cancer.
131. A compound according to any one of paragraphs 1 to 125, or a deuterated form, a pharmaceutically acceptable salt, hydrate or solvate thereof, or a pharmaceutical composition according to paragraph 126, for use in the treatment or prevention of a PRMT5-mediated disorder.
132. Use of a compound according to any one of paragraphs 1 to 125, or a deuterated form, a pharmaceutically acceptable salt, hydrate or solvate thereof, or a pharmaceutical composition according to paragraph 126, in the manufacture of a medicament for the treatment or prevention of a PRMT5-mediated disorder.
133. Use of a compound according to any one of paragraphs 1 to 125, or a deuterated form, a pharmaceutically acceptable salt, hydrate or solvate thereof, or a pharmaceutical composition according to paragraph 126, in the manufacture of a medicament for the treatment of a proliferative disorder.
134. Use of a compound according to any one of paragraphs 1 to 125, or a deuterated form, a pharmaceutically acceptable salt, hydrate or solvate thereof, or a pharmaceutical composition according to paragraph 126, in the manufacture of a medicament for the treatment of cancer.
135. Use according to paragraph 134, wherein the cancer is selected from colorectal, ovarian, prostate, lung, breast, lymphoma/leukaemia, oesophageal, gastric, hepatocellular and brain cancer.
136. A method of treating or preventing a PRMT5-mediated disorder, said method comprising administering to a subject in need thereof an effective amount of a compound according to any one of paragraphs 1 to 125, or a deuterated form, a pharmaceutically acceptable salt, hydrate or solvate thereof, or a pharmaceutical composition according to paragraph 126.
137. A method of treating a proliferative disorder, said method comprising administering to a subject in need thereof an effective amount of a compound according to any one of paragraphs 1 to 125, or a deuterated form, a pharmaceutically acceptable salt, hydrate or solvate thereof, or a pharmaceutical composition according to paragraph 126.
138. A method of treating cancer, said method comprising administering to a subject in need thereof a therapeutically effective amount of a compound according to any one of paragraphs 1 to 125, or a deuterated form, a pharmaceutically acceptable salt, hydrate or solvate thereof, or a pharmaceutical composition according to paragraph 126.
139. A method of inhibiting the activity of PRMT5 in vivo or in vitro. When the method is an in vivo or in vitro method, then the method may comprise contacting a cell with an effective amount of a compound according to any one of paragraphs 1 to 125, or a deuterated form, a pharmaceutically acceptable salt, hydrate or solvate thereof, or a pharmaceutical composition according to paragraph 126. When the method is an in vivo method, then additionally or alternatively the method may comprise administering to a subject in need thereof a therapeutically effective amount of a compound according to any one of paragraphs 1 to 125, or a deuterated form, a pharmaceutically acceptable salt, hydrate or solvate thereof, or a pharmaceutical composition according to paragraph 126.
140. A method of altering gene expression in a cell which comprises contacting a cell with an effective amount of a compound according to any one of paragraphs 1 to 125, or a deuterated form, a pharmaceutically acceptable salt, hydrate or solvate thereof, or a pharmaceutical composition according to paragraph 126.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In an aspect of the invention, the compounds of formula (1), (1′) or (1″) are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of formula (1), (1′) or (1″) may exist in solvated as well as unsolvated forms such as, for example, hydrated forms. It is to be understood that the invention encompasses all such solvated forms that possess antiproliferative activity.

### Polymorphs

It is also to be understood that certain compounds of the formula (1), (1′) or (1″) may exhibit polymorphism, and that the invention encompasses all such forms that possess antiproliferative activity.

### N-oxides

Compounds of the formula (1), (1′) or (1″) containing an amine function may also form N-oxides. A reference herein to a compound of the Formula (1), (1′) or (1″) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

### Tautomers

Compounds of the formula (1), (1′) or (1″) may exist in a number of different tautomeric forms and references to compounds of the formula (1), (1′) or (1″) include all such forms. For the avoidance of doubt, where a compound can exist in one of several tautomeric forms, and only one is specifically described or shown, all others are nevertheless embraced by Formula (1), (1′) or (1″). Examples of tautomeric forms include keto-, enol-, and enolate-forms, as in, for example, the following tautomeric pairs: keto/enol (illustrated below), pyrimidone/hydroxypyrimidine, imine/enamine, amide/imino alcohol, amidine/amidine, nitroso/oxime, thioketone/enethiol, and nitro/aci-nitro.

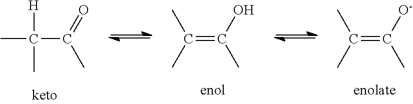

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Certain compounds of formula (1), (1′) or (1″) may have one or more asymmetric centers and therefore can exist in a number of stereoisomeric configurations. Consequently, such compounds can be synthesized and/or isolated as mixtures of enantiomers and/or as individual (pure) enantiomers, and, in the case of two or more asymmetric centers, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Prodrugs and Metabolites

The compounds of formula (1), (1′) or (1″) may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula (1), (1′) or (1″) and in-vivo cleavable amide derivatives that may be formed at a carboxy group or an amino group in a compound of the formula (1), (1′) or (1″).

Accordingly, the present invention includes those compounds of the formula (1), (1′) or (1″) as defined hereinbefore when made available by organic synthesis and when made available within the human or animal body by way of cleavage of a pro-drug thereof. Accordingly, the present invention includes those compounds of the formula (1), (1′) or (1″) that are produced by organic synthetic means and also such compounds that are produced in the human or animal body by way of metabolism of a precursor compound, that is a compound of the formula (1), (1′) or (1″) may be a synthetically-produced compound or a metabolically-produced compound.

A suitable pharmaceutically acceptable pro-drug of a compound of the formula (1), (1′) or (1″) is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula (1), (1′) or (1″) that possesses a carboxy group is, for example, an in vivo cleavable ester thereof. An in vivo cleavable ester of a compound of the formula (1), (1′) or (1″) containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula (1) or (1′) that possesses a hydroxy group is, for example, an in vivo cleavable ester or ether thereof. An in vivo cleavable ester or ether of a compound of the formula (1), (1′) or (1″) containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula (1), (1′) or (1″) that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula (1), (1′) or (1″) that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula (1), (1′) or (1″) may be exerted in part by one or more metabolites that are formed within the human or animal body after administration of a compound of the formula (1), (1′) or (1″). As stated hereinbefore, the in vivo effects of a compound of the formula (1), (1′) or (1″) may also be exerted by way of metabolism of a precursor compound (a pro-drug).

### Pharmaceutical Compositions

According to a further aspect of the invention there is provided a pharmaceutical composition which comprises a compound of the invention as defined hereinbefore, or a deuterated form, pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the Formula (1) or (1′) will naturally vary according to the nature and severity of the conditions, the age and sex of the animal or patient and the route of administration, according to well-known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The invention provides a compound of formula (1), (1′) or (1″) as defined herein, or a deuterated form, pharmaceutically acceptable salt or solvate thereof, or a pharmaceutical composition as defined herein, for use in therapy and/or for use as a medicament.

The invention provides a compound of formula (1), (1′) or (1″) as defined herein, or a deuterated form, pharmaceutically acceptable salt or solvate thereof, or a pharmaceutical composition as defined herein, for use in the treatment or prevention of a PRMT5-mediated disorder.

Also provided by the invention is a compound of formula (1), (1′) or (1″) as defined herein, or a deuterated form, pharmaceutically acceptable salt or solvate thereof, or a pharmaceutical composition as defined herein, for use in the treatment of a proliferative disorder.

The invention further provides a compound of formula (1), (1′) or (1″) as defined herein, or a deuterated form, pharmaceutically acceptable salt or solvate thereof, or a pharmaceutical composition as defined herein, for use in the treatment cancer.

In a further aspect, the invention provides the use of a compound of formula (1), (1′) or (1″) as defined herein, or a deuterated form, pharmaceutically acceptable salt or solvate thereof, in the manufacture of a medicament for the treatment or prevention of a PRMT5-mediated disorder.

In another aspect, the invention provides the use of a compound of formula (1), (1′) or (1″) as defined herein, or a deuterated form, pharmaceutically acceptable salt or solvate thereof, in the manufacture of a medicament for the treatment of a proliferative disorder.

The invention also provides the use of a compound of formula (1), (1′) or (1″) as defined herein, or a deuterated form, pharmaceutically acceptable salt or solvate thereof, in the manufacture of a medicament for the treatment of cancer.

In another aspect, the invention provides a method of treating or preventing a PRMT5-mediated disorder, said method comprising administering to a subject in need thereof an effective amount of a compound of formula (1), (1′) or (1″) as defined herein, or a deuterated form, pharmaceutically acceptable salt or solvate thereof.

In another aspect, the present invention provides a method of treating a proliferative disorder, said method comprising administering to a subject in need thereof an effective amount of a compound of formula (1), (1′) or (1″) as defined herein, or a deuterated form, pharmaceutically acceptable salt or solvate thereof.

The invention also provides a method of treating cancer, said method comprising administering to a subject in need thereof a therapeutically effective amount of a compound of formula (1), (1′) or (1″) as defined herein, or a deuterated form, pharmaceutically acceptable salt or solvate thereof, or a pharmaceutical composition as defined herein.

The invention further provides a method of inhibiting the activity of PRMT5 in vivo or in vitro, said method comprising administering to a subject in need thereof a therapeutically effective amount of a compound of formula (1), (1′) or (1″) as defined herein, or a deuterated form, pharmaceutically acceptable salt or solvate thereof, or a pharmaceutical composition as defined herein.

In another aspect, the invention provides a combination comprising a compound of formula (1), (1′) or (1″), or a deuterated form, pharmaceutically acceptable salt or solvate thereof, as defined herein, with one or more additional therapeutic agents.

In another aspect, the invention provides a method of altering gene expression in a cell which comprises contacting a cell with an effective amount of a compound of formula (1), (1′) or (1″) as defined herein, or a deuterated form, pharmaceutically acceptable salt or solvate thereof, or a pharmaceutical composition as defined herein.

As a general feature of the invention, the PRMT5 disorder may be selected from a proliferative disorder, a metabolic disorder or a blood disorder. Suitably, the PRMT5 disorder is a proliferative disorder or a metabolic disorder.

Suitably the blood disorder is sickle cell disease or β-thalassemia.

Suitably the metabolic disorder is diabetes or obesity.

Suitably the proliferative disorder is cancer, an autoimmune disorder or an inflammatory disorder. Suitably, the proliferative disorder is cancer.

In general, the cancer may be selected from breast cancer, esophageal cancer, bladder cancer, lung cancer, hematopoietic cancer, lymphoma, medulloblastoma, rectum adenocarcinoma, colon adenocarcinoma, gastric cancer, pancreatic cancer, liver cancer, adenoid cystic carcinoma, lung adenocarcinoma, head and neck squamous cell carcinoma, brain tumors, hepatocellular carcinoma, renal cell carcinoma, melanoma, oligodendroglioma, ovarian clear cell carcinoma, and ovarian serous.

Suitably, the cancer is selected from breast cancer, esophageal cancer, bladder cancer, lung cancer, hematopoietic cancer, lymphoma, medulloblastoma, rectum adenocarcinoma, colon adenocarcinoma, gastric cancer, pancreatic cancer, liver cancer, head and neck squamous cell carcinoma and brain tumors.

It may be preferable that the cancer is a cancer in which high expression of PRMT5 occurs. Examples of such cancers include colorectal, ovarian, prostate, lung, breast, lymphoma/leukaemis, oesophageal, gastric, hepatocellular and brain cancer.

### Route of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The treatment defined hereinbefore may be applied as a sole therapy or may involve, in addition to the compound of the invention, conventional surgery or radiotherapy or chemotherapy. Such chemotherapy may include one or more of the following categories of anti-tumour agents:—
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin avB3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of the invention, conventional surgery or radiotherapy or chemotherapy.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention, there is provided a combination for use in the treatment of a cancer (for example a cancer involving a solid tumour) comprising a compound of the invention as defined hereinbefore, or a deuterated form, pharmaceutically acceptable salt, hydrate or solvate thereof, and another anti-tumour agent.

According to this aspect of the invention, there is provided a combination for use in the treatment of a proliferative condition, such as cancer (for example a cancer involving a solid tumour), comprising a compound of the invention as defined hereinbefore, or a deuterated form, pharmaceutically acceptable salt, hydrate or solvate thereof, and any one of the anti-tumour agents listed herein above.

In a further aspect of the invention there is provided a compound of the invention or a deuterated form, pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. In one embodiment, a combination refers to a combination product.

According to a further aspect of the invention there is provided a combination comprising a compound of formula (1), (1′) or (1″) as defined herein, or a deuterated for, pharmaceutically acceptable salt, hydrate or solvate thereof, in combination with an further therapeutic agent (optionally selected from one listed herein above.

In one embodiment, there is provided a pharmaceutical composition which comprises a compound of formula (1), (1′) or (1″), or a deuterated form, pharmaceutically acceptable salt, hydrate or solvate thereof, in combination with a therapeutic agent (optionally selected from one listed herein above), in association with a pharmaceutically acceptable diluent or carrier.

Suitably, the additional therapeutic agent is an anti-cancer agent (optionally selected from one listed herein above).

### EXAMPLES

The following examples are provided to illustrate the invention and are not intended to limit the scope of the invention, as described herein.

The compounds of the invention may be prepared using synthetic techniques that are known in the art (as illustrated by the examples herein).

Several methods for the chemical synthesis of the compounds of the present application are described herein. These and/or other well-known methods may be modified and/or adapted in various ways in order to facilitate the synthesis of additional compounds within the scope of the present application and claims. Such alternative methods and modifications should be understood as being within the spirit and scope of this application and claims. Accordingly, it should be understood that the methods set forth in the following descriptions, schemes and examples are intended for illustrative purposes and are not to be construed as limiting the scope of the disclosure.

Methods of Analysis used in Synthetic Examples

### LCMS Method

### Method-A

Method Name: UC04_FAR1

### LC Parameters

Instruments: Waters Acquity Ultra performance LC equipped with PDA and attached with QDA detector
Column: X—BRIDGE BEH C18 2.5 μm 2.1*50 mm
Mobile phase: (A) 0.1% Formic acid in milli Q water (pH=2.70)
   (B) Acetonitrile: 0.1% Formic acid (90:10)
Flow rate: 0.8 mL/min
Column temperature: 35° C.
Auto sampler
temperature: 5° C.
Run time: 4 min
Gradient:

| Time (minute) | %(A) | %(B) |
|---|---|---|
| 0.00 | 97 | 3 |
| 0.75 | 97 | 3 |
| 2.70 | 2 | 98 |
| 3.00 | 0 | 100 |
| 3.50 | 0 | 100 |
| 3.51 | 97 | 3 |
| 4.00 | 97 | 3 |

### Mass Parameters

Probe: ESI Capillary
Source Temperature: 120° ° C.
Probe Temperature: 600° C.
Capillary Voltage: 0.8 kv (+Ve and −Ve)
Cone Voltage: 10 & 30V
Mode of Ionization: +Ve and −Ve

### Method-B

Method Name: UC03_ABR2

### LC Parameters

Instruments: Waters Acquity Ultra performance LC connected with PDA and equipped with SQ detector
Column: XTIMATE C18 5 μm 4.6*50 mm
Mobile phase: (A) 5 mM % ammonium bicarbonate in milli Q water (pH=7.35)
   (B) Acetonitrile
Flow rate: 1 mL/min
Column temperature: 35° C.
Auto sampler
temperature: 5° ° C.
Run time: 4 min
Gradient:

| Time (minute) | %(A) | %(B) |
|---|---|---|
| 0.00 | 97 | 3 |
| 0.20 | 97 | 3 |
| 2.70 | 20 | 80 |
| 3.00 | 0 | 100 |
| 3.50 | 0 | 100 |
| 3.51 | 97 | 3 |
| 4.00 | 97 | 3 |

### Mass Parameters

Probe: ESI Capillary
Source Temperature: 120° C.
Probe Temperature: 400° C.
Capillary Voltage: 3 kv (+Ve and −Ve)
Cone Voltage: 10 & 30V
Mode of Ionization: +Ve and −Ve

### HPLC Method

Method name: HP07_TFAR1

### LC Parameters

Column: Xtimate C18, 150*4.6 mm, 5.0 μm
Mobile Phase: (A) 0.05% Trifloro acetic acid in Milli-Q Water
   (B) Acetonitrile
Flow Rate: 1.0 ml/min
Column Temperature: 30° C.
Auto sampler: 15° C.
Run Time: 17 min
Gradient:

| Time (minute) | (%)A | (%)B |
|---|---|---|
| 0.00 | 90 | 10 |
| 7.00 | 10 | 90 |
| 9.00 | 00 | 100 |
| 14.00 | 00 | 100 |
| 14.01 | 90 | 10 |
| 17.00 | 90 | 10 |

### Chiral HPLC Method

Instrument: SHIMADZU LC-20 AD
Column: CHIRAL PAK IG, 250*4.6 mm, 5 μm
Mobile Phase: (A) 0.1% v/v DEA in MeOH
   (B) ACN
Sample Preparation: Approx. 1000 PPM in Mobile Phase
Flow Rate: 1 ml/Min
Column Temperature: Room temperature
Auto sampler
temperature: Room temperature
Run time: 25 min
Isocratic: A:B (20-80)

### Chiral Prep HPLC Method

Instrument: Agilent PHP-04
Column: CHIRALPAK IG SFC, 21×250 mm, 5 μm
Mobile Phase: (A) ACN
   (B) 0.1% DEA in MeOH
Sample preparation: 25 mg in MeOH:ACN
Flow Rate: 25 mL/min
Runtime: 40 min/injection

### Synthesis of Compounds

The compounds were prepared from commercially available reagents using the general reaction scheme shown in Scheme 1.

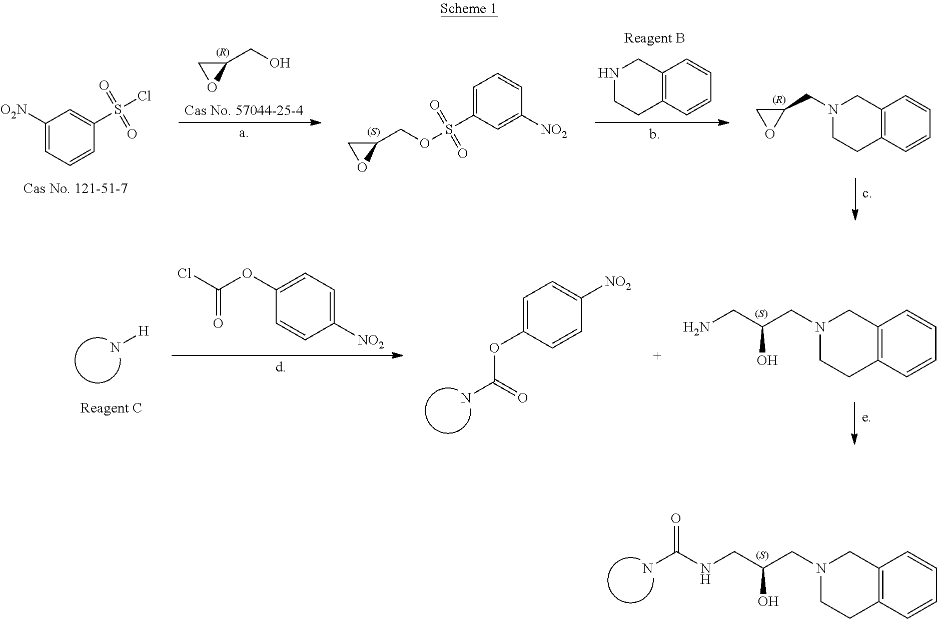

Reagents and conditions: (a) triethylamine, dichloromethane, 0-5° C., 1 h (b) triethylamine, dichloromethane, RT, 16 h (c) NH₃ gas purging at −78° C. followed by heating under hydrogenator at 55° C. for 6 h (d) diisopropylethylamine, THF, RT, 1 h (e) K₂CO₃, DMF, 60° ° C., 48-50 h.

The compounds can also be preparing using a method as described in WO 2018/167269.

### Synthesis of (S)-1-amino-3-(3,4-dihydroisoquinolin-2(1H)-yl)propan-2-ol (Int-A3)

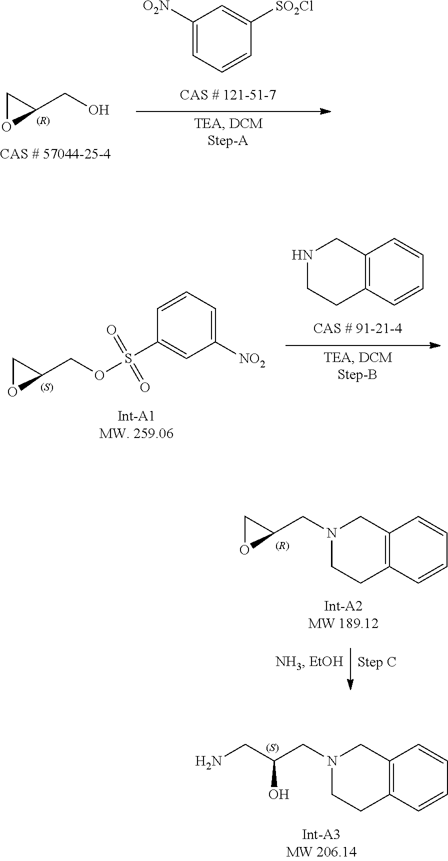

### Step-A: (S)-oxiran-2-ylmethyl 3-nitrobenzenesulfonate

To a solution of (R)-oxiran-2-ylmethanol (CAS No. 57044-25-4) (50.0 g, 0.675 mol) in dichloromethane (500 mL) was added TEA (113 mL, 0.810 mol) at 0° C. The reaction mixture was stirred at same temperature for 10 min. Further 3-nitrobenzenesulphonyl chloride (149.3 g, 0.675 mol) was added portion wise over a period of 1.5 h to the reaction mixture at 0° C. The resulting reaction mixture was stirred at ambient temperature for 15 min. The resulting reaction mixture was poured into water (500 mL) and then extracted with dichloromethane (500 mL×2). The combined organic layers were dried over anhydrous sodium sulphate, filtered and concentrated under reduced pressure to yield (S)-oxiran-2-ylmethyl 3-nitrobenzenesulfonate (Int-A1) (159 g, Yield Qnt.) as a yellow oil, which was used in the next step without further purification. LCMS: 82.20% at 1.580 min (Method A).

### Step-B: (R)-2-(oxiran-2-ylmethyl)-1,2,3,4-tetrahydroisoquinoline

To a solution of (S)-oxiran-2-ylmethyl 3-nitrobenzenesulfonate (159 g, 0.613 mmol) in dichloromethane (500 mL) was added TEA (124 mL, 0.920 mol). The reaction mixture was stirred at the same temperature for 20 min. Further solution of 1,2,3,4-tetrahydroisoquinoline (81.64 g, 0.613 mol) in dichloromethane (100 mL) was added drop wise over a period of 2 h and resulting reaction mixture was stirred at ambient temperature for 18 h.

After completion, to the resulting reaction mixture was added a solution of citric acid (until pH 4 was obtained) and then the aqueous layer was washed with ethyl acetate to remove other impurities. The aqueous layer was then neutralized with sat. NaHCO₃ and then extracted with diethyl ether (400 mL×3). The combined organic layers were dried over anhydrous sodium sulphate, filtered and concentrated under reduced pressure to yield (R)-2-(oxiran-2-ylmethyl)-1,2,3,4-tetrahydroisoquinoline (Int-A2) (55 g, Yield—47.41%) as a yellow oil, which was used in the next step without further purification.

### Step-C: (S)-1-amino-3-(3,4-dihydroisoquinolin-2(1H)-yl)propan-2-ol

A solution of (R)-2-(oxiran-2-ylmethyl)-1,2,3,4-tetrahydroisoquinoline (55 g, 0.291 mol) in EtOH (100 mL) was charged in single neck RBF which was cooled to −78° C. and resulting solution was purged using ammonia (volume increased to ˜200 mL after ammonia purging) for 1 h. After completion of ammonia purging the whole reaction mixture was transferred to 1 L autoclave and heated at 55° C. for 6 h.

After completion, reaction mixture was cooled to −30° C. and a pressure of NH₃ was released. Further reaction mixture was concentrated under reduced pressure. The resulting crude material was purified by reverse phase chromatography using water:ACN and 0.1% NH₃ as buffer to yield (S)-1-amino-3-(3,4-dihydroisoquinolin-2(1H)-yl)propan-2-ol (Int-A3) (22 g, 36.70%) as a brown oil. LCMS: 93.93% at 2.38 min, (M+1 in basic method) Chiral HPLC: RT 11.043 93.86%.

### Example 1

### Preparation of (R)—N—((S)-3-(3,4-dihydroisoquinolin-2(1H)-yl)-2-hydroxypropyl)-3-(2-oxopyrrolidin-1-yl)piperidine-1-carboxamide

The synthetic steps shown in the reaction scheme below were used.

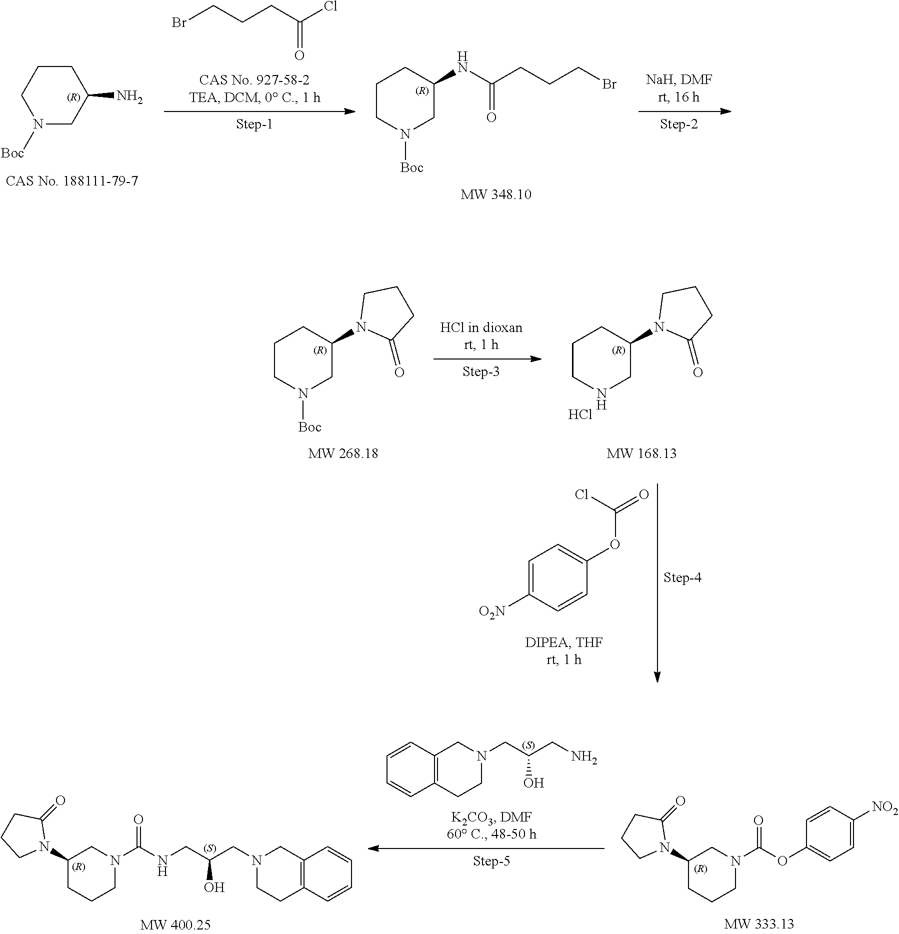

### Step-1: Synthesis of tert-butyl (R)-3-(4-bromobutanamido)piperidine-1-carboxylate

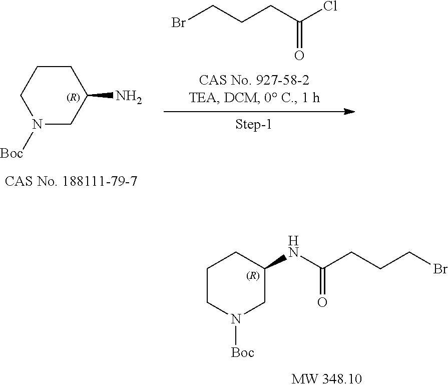

In an oven dried single neck round bottom flask was charged a solution of CAS No. 18811-79-7 (40 g, 0.2 mol) in dichloromethane (10 v, 400 mL). The reaction mixture was cooled to 0° C. and triethylamine (24.24 g, 0.24 mol) was added. After 15 min of stirring, CAS No. 927-58-2 (40.57 g, 0.22 mol) was added dropwise over period of 15 min. The reaction mixture was allowed to stir at room temperature for 1-2 h. The reaction mixture was diluted with water (300 mL) and extracted with dichloromethane (500 mL×2). The combined organic layer was dried over anhydrous sodium sulphate, filtered and concentrated under reduced pressure to afford 56 g of tert-butyl (R)-3-(4-bromobutanamido)piperidine-1-carboxylate (56 g, Yield Qnt.) as a thick, brown oil. LCMS purity 81.45% (Method A).

### Step-2: Synthesis of tert-butyl (R)-3-(2-oxopyrrolidin-1-yl)piperidine-1-carboxylate

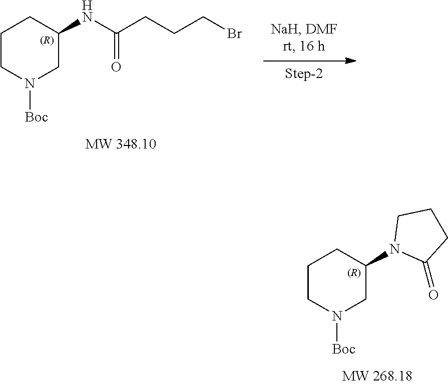

NaH (3.72 g, 0.093 mol, 60% in mineral oil) was suspended in 100 mL DMF in a single neck round bottomed flask and was cooled to 0° C. A solution of tert-butyl (R)-3-(4-bromobutanamido)piperidine-1-carboxylate (27 g, 0.077 mol) in 100 mL DMF was added dropwise over a 30 min period. The resulting reaction mixture was stirred at room temperature for 16 h. The reaction mixture was then diluted with cold water (200 mL) and extracted in ethyl acetate (400 mL×2). The combined organic layer was dried over anhydrous sodium sulphate, filtered and concentrated under reduced pressure to produce a crude material. The resulting crude material was further purified by column chromatography using 0-2% MeOH:DCM to afford (17 g, yield 81.7%) tert-butyl (R)-3-(2-oxopyrrolidin-1-yl)piperidine-1-carboxylate as a brown sticky liquid. LCMS purity 100% (Method A).

### Step-3: (R)-1-(piperidin-3-yl)pyrrolidin-2-one

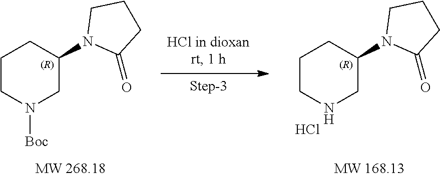

To a stirred solution of tert-butyl (R)-3-(2-oxopyrrolidin-1-yl)piperidine-1-carboxylate (17 g, 0.063 mol) in DCM (100 ml) was added 4N HCl in dioxane (100 ml) dropwise and the reaction was stirred at room temperature for 2 h. The reaction mixture was directly evaporated under reduced pressure to afford (R)-1-(piperidin-3-yl)pyrrolidin-2-one (19 g, Yield Qnt) as a white sticky solid. LCMS purity 98.02% (Method B).

### Step-4: Synthesis 4-nitrophenyl (R)-3-(2-oxopyrrolidin-1-yl)piperidine-1-carboxylate CAS #7693-46-1

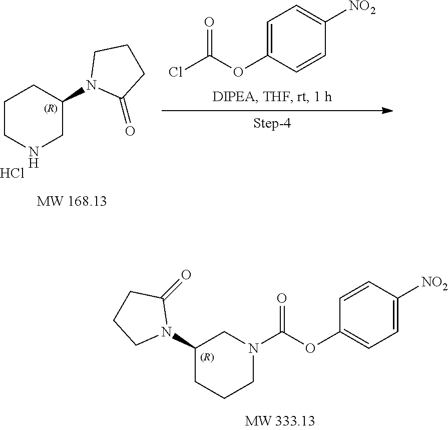

To a stirred solution of (R)-1-(piperidin-3-yl)pyrrolidin-2-one 19 g, 0.113 mol) in THF (150 ml) was added DIPEA (22.73 g, 0.113 mol) at room temperature. After 15 min stirring, CAS #7693-46-1 (22.73 g. 0.113 mol) was added portion wise and the reaction mixture was then stirred at room temperature for 1 h. The reaction mixture was diluted with water (200 mL) and extracted with ethyl acetate (200 mL×2). The combined organic layer was dried over anhydrous sodium sulphate, filtered and concentrated under reduced pressure to afford a crude material. The resulting crude material was purified by column chromatography using 0-2% methanol in DCM to afford 4-nitrophenyl (R)-3-(2-oxopyrrolidin-1-yl)piperidine-1-carboxylate (10 g, Yield-26.55%) as an off white solid. LCMS purity 96.55% (Method B).

### Step-5: Synthesis of (R)—N—((S)-3-(3,4-dihydroisoquinolin-2(1H)-yl)-2-hydroxypropyl)-3-(2-oxopyrrolidin-1-yl)piperidine-1-carboxamide

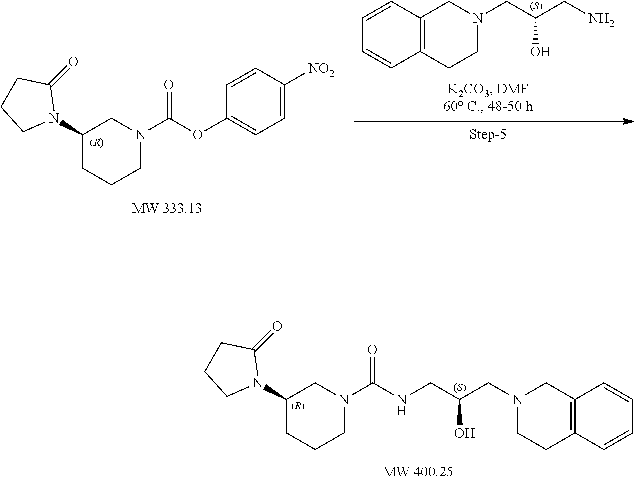

To a stirred solution of 4-nitrophenyl (R)-3-(2-oxopyrrolidin-1-yl)piperidine-1-carboxylate (3 g, 0.006 ml) in DMF (30 mL) was added (S)-1-amino-3-(3,4-dihydroisoquinolin-2(1H)-yl)propan-2-ol (2.78 g, 0.013 mol) and potassium carbonate (4.97 g, 0.036 mol) at room temperature. The reaction mixture was then heated at 65° C. for 48-50 h. The reaction mixture was diluted with ice cold water (100 mL) and extracted with ethyl acetate (200 mL×2). The combined organic layer was washed with brine (20 mL×2). The organic layer was dried over anhydrous sodium sulphate, filtered and concentrated under reduced pressure. The obtained crude product was purified by reverse phase flash chromatography using 0.1% formic acid solution in water:acetonitrile to afford (R)—N—((S)-3-(3,4-dihydroisoquinolin-2(1H)-yl)-2-hydroxypropyl)-3-(2-oxopyrrolidin-1-yl)piperidine-1-carboxamide (1.4 g, yield 38.88%) as a yellow sticky solid. LCMS purity 97.58% (Method A). 1H NMR 400 MHZ, DMSO-d₆: 8.155 (s, 1H, formic salt), 7.102-7.026 (m, 4H), 6.624-6.599 (t, 1H), 3.812-3.762 (m, 3H), 3.669-3.543 (m, 4H), 3.321-3.223 (m, 4H), 3.194-3.181 (m, 1H), 3.161-3.135 (m, 1H), 2.803-2.790 (m, 2H), 2.759-2.708 (m, 3H), 2.443-2.411 (m, 2H), 2.219-2.199 (t, 2H), 1.884-1.849 (q, 2H), 1.592-1.509 (m, 3H), 1.388-1.332 (m, 1H).

### Example 2

### Experimental Preparation of (R)—N—((S)-3-(3,4-dihydroisoquinolin-2(1H)-yl)-2-hydroxypropyl)-2-oxo-[1,3′-bipiperidine]-1′-carboxamide

The synthetic steps shown in the reaction scheme below were performed.

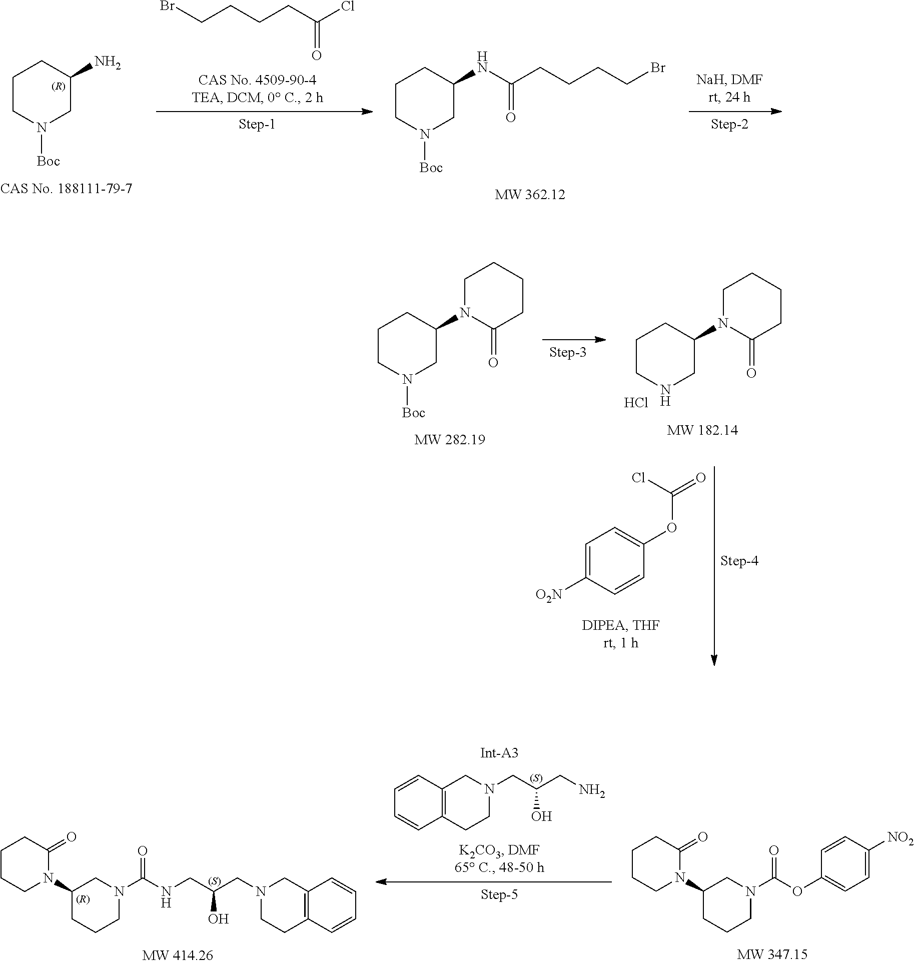

### Step-1: Synthesis of tert-butyl (R)-3-(5-bromopentanamido) piperidine-1-carboxylate

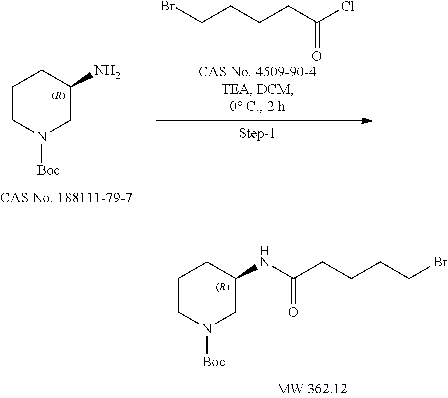

In an oven dried single neck round bottom flask was charged a solution of CAS No. 188111-79-7 (50 g, 0.249 mol) in dichloromethane (10 v, 500 mL). The reaction mixture was cooled to 0° C. and triethylamine was added. After 15 min of stirring, CAS No. 4509-90-4 (54.14 g, 0.274 mol) was added dropwise over period of 30 min. The reaction mixture was allowed to stir at RT for 1-2 h. Reaction progress was monitored using TLC (mobile phase: neat ethyl acetate). CAS No. 188111-79-7 shows consumed over period of 2 h. (Another batch of 25 g using same stoichiometry was kept and combined during work-up). The reaction mixture was diluted with water (3000 mL) and extracted with dichloromethane (3000 mL×2). The combined organic layer was dried over anhydrous sodium sulphate, filtered and concentrated under reduce pressure to afford 135 g of tert-butyl (R)-3-(5-bromopentanamido) piperidine-1-carboxylate (135 g, Yield Qnt.) as a brown thick oil. LCMS purity 78.26% (Method A).

### Step-2: Synthesis of tert-butyl (R)-2-oxo-[1,3′-bipiperidine]-1′-carboxylate

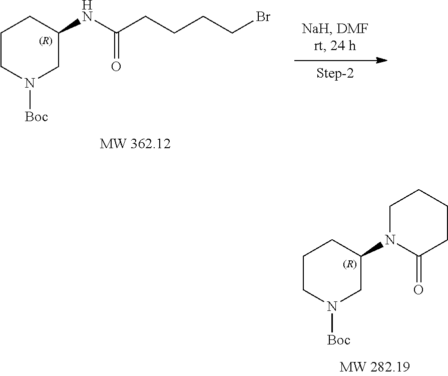

To a stirred suspension of 60% sodium hydride in mineral oil (12.72 g, 0.265 mol) in DMF (100 mL) was added solution of tert-butyl (R)-3-(5-bromopentanamido) piperidine-1-carboxylate (96 g, 0.318 mol) in DMF (100 mL) at 0° C. over period of 0.5 h. The resulting reaction mixture was stirred at room temperature for 24 h. The reaction progress was monitored using TLC (neat ethyl acetate). After consumption of tert-butyl (R)-3-(5-bromopentanamido) piperidine-1-carboxylate, the reaction mixture was quenched with ice cold water (1000 mL) and extracted with ethyl acetate (3000 mL×2). The combined organic layer was dried over anhydrous sodium sulphate, filtered and concentrated under reduced pressure. The obtained crude was purified by silica gel (60-120 mesh) column chromatography using 0-2% methanol in dichloromethane to afford tert-butyl (R)-2-oxo-[1,3′-bipiperidine]-1′-carboxylate (56 g, Yield 74.88%) as a brown thick oil. LCMS purity 98.65% (Method A).

### Step-3: Synthesis of (R)-[1,3′-bipiperidin]-2-one

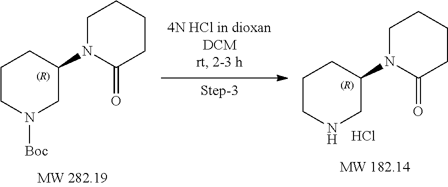

To a stirred solution of tert-butyl (R)-2-oxo-[1,3′-bipiperidine]-1′-carboxylate (56 g, 0.198 mol) in DCM (150 mL) was added 4N HCl in dioxane (150 mL) at room temperature. The reaction mixture was allowed to stir at room temperature for 2 h. The reaction mixture was then directly concentrated under reduced pressure to afford (R)-[1,3′-bipiperidin]-2-one (70 g, Yield Qnt.) as a white sticky solid. The isolated solid was used as such for next stage.

### Step-4: Synthesis of 4-nitrophenyl (R)-2-oxo-[1,3′-bipiperidine]-1′-carboxylate

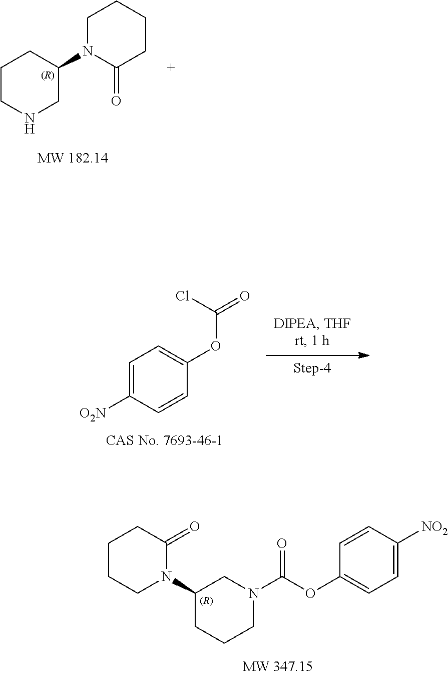

To a stirred solution of (R)-[1,3′-bipiperidin]-2-one (70 g, 0.082 mol) in THF (200 mL) was added DIPEA (148.84 g, 0.384 mol) at room temperature, followed by 4-nitrophenyl chloroformate (77.3 g, 0.384 mol). The reaction mixture was allowed to stir at room temperature for 1 h. The reaction mixture was then diluted with water (2000 mL) and extracted with ethyl acetate (2000 mL×2). The combined organic layer was dried over anhydrous sodium sulphate, filtered and concentrated under reduced pressure. The obtained crude was purified by silica gel (60-120 mesh) column chromatography using 0-2% methanol in dichloromethane to afford 4-nitrophenyl (R)-2-oxo-[1,3′-bipiperidine]-1′-carboxylate (37 g, Yield 27.74%) as a white solid. LCMS purity 99.39% (Method A).

### Step-5: Synthesis of (R)—N—((S)-3-(3,4-dihydroisoquinolin-2(1H)-yl)-2-hydroxypropyl)-2-oxo-[1,3′-bipiperidine]-1′-carboxamide

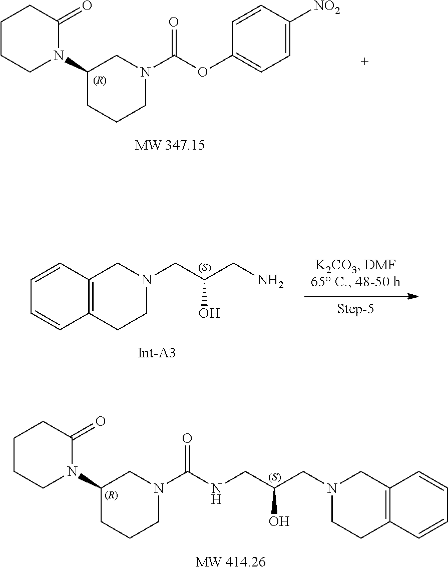

To a stirred solution of 4-nitrophenyl (R)-2-oxo-[1,3′-bipiperidine]-1′-carboxylate (2.0 g, 0.0057 mol) in DMF (150 mL) was added (S)-1-amino-3-(3,4-dihydroisoquinolin-2(1H)-yl)propan-2-ol (Int-A3) (1.78 g, 0.0086 mol) and potassium carbonate (2.38 g, 0.017 mol) at room temperature. The reaction mixture was heated at 65° C. for 48-50 h. The reaction progress was monitored using TLC (mobile phase: 5% MeOH in DCM under NH₃ atmosphere). A total of 9 reactions were carried out in parallel manner using above calculations and combined during work-up. After consumption of 4-nitrophenyl (R)-2-oxo-[1,3′-bipiperidine]-1′-carboxylate, the reaction mixture was diluted with ice cold water (1000 mL) and extracted with ethyl acetate (1000 mL×2). Combined organic layer was washed with chilled brine (1000 mL×3). Organic layer was dried over anhydrous sodium sulphate, filtered and concentrated under reduced pressure. The obtained crude was purified by reverse phase flash chromatography using 0.1% ammonia solution in water:acetonitrile to afford (R)—N—((S)-3-(3,4-dihydroisoquinolin-2(1H)-yl)-2-hydroxypropyl)-2-oxo-[1,3′-bipiperidine]-1′-carboxamide (10.242 g, Yield 50.50%). LCMS purity 97.58% (Method A). 1H NMR: 400 MHZ, DMSO-d₆: 7.11-7.01 (4H, m), 6.58-6.56 (t, 1H), 4.83-4.82 (d, J=4 Hz, 1H), 4.16-4.10 (m, 1H), 3.816-3.775 (m, 3H), 3.641-3.601 (q, 2H), 3.187-3.110 (m, 3H), 3.03-2.981 (m, 1H), 2.791-2.560 (m, 5H), 2.459-2.446 (m, 1H), 2.409-2.362 (m, 1H), 2.235-2.205 (t, 2H), 1.660-1.582 (m, 7H), 1.352-1.320 (m, 1H).

### Example 3

### Preparation of (3′R)—N—(S)-3-(3,4-dihydroisoquinolin-2(1H)-yl)-2-hydroxypropyl)-5-methyl-2-oxo-[1,3′-bipiperidine]-1′-carboxamide

The synthetic steps shown in the reaction scheme below were performed.

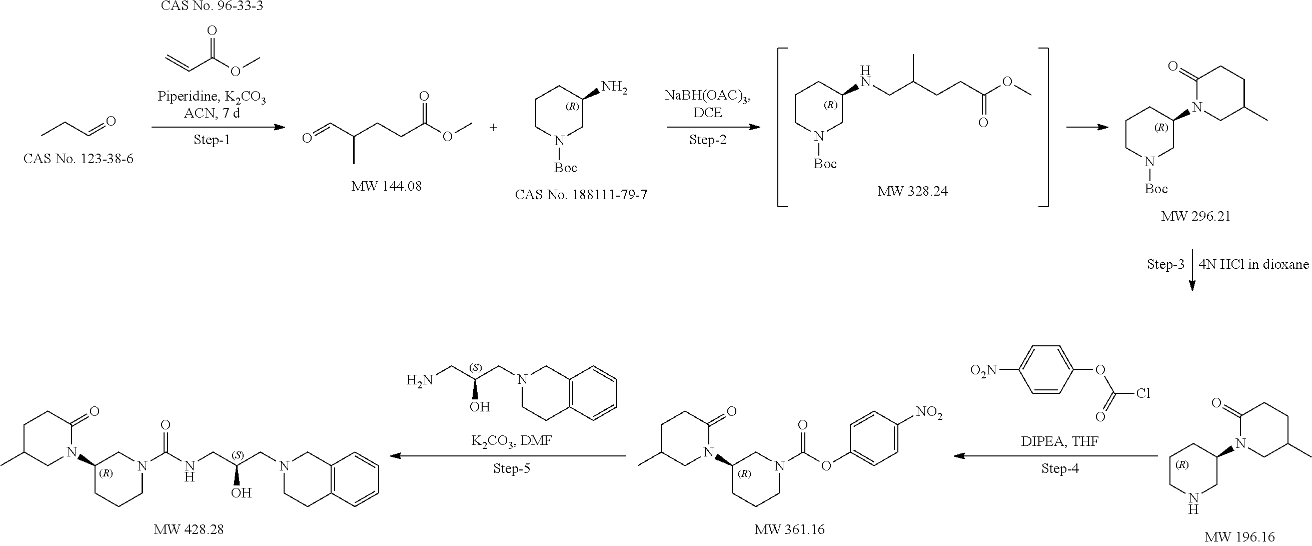

### Step-1: Synthesis of methyl 4-methyl-5-oxopentanoate

### CAS No 96-33-3

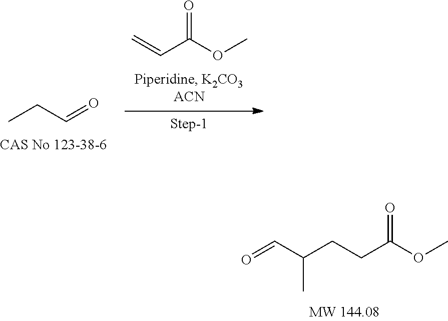

In an oven dried single neck round bottom flask was charged K₂CO₃ (4.1 g, 0.35 mol) in piperidine (14.6 g, 0.172 mol) and cooled to 0° C. After 10 min of stirring, CAS No. 123-38-6 (5 g, 0.086 mol) was added and resulting mixture was stirred at room temperature for 24 h. The resulting mixture was then filtered through a celite pad and the filtrate was concentrated. The resulting crude material was then dissolved in 25 mL acetonitrile and methyl acrylate (14.8 g, 0.172 mol) was added dropwise to it. The resulting solution was then stirred for 12 h at room temperature and then refluxed for 24 h. After 24 h of refluxing AcOH (2.5 ml) and Water (2.5 ml) was added to it and further refluxed for 2 days. The reaction mixture was diluted with water (50 mL) and extracted with ethyl acetate (100 mL). Organic layer was then washed with sat. sodium bi-carbonate solution and dried over anhydrous sodium sulphate, filtered and concentrated under reduced pressure to get crude material. The resulting crude material was purified by column chromatography using 30% EA:hexane to afford 5.5 g as a light yellow liquid. LCMS purity 91.30% (Method A).

### Step-2: Synthesis of tert-butyl (3′R)-5-methyl-2-oxo-[1,3′-bipiperidine]-1′-carboxylate

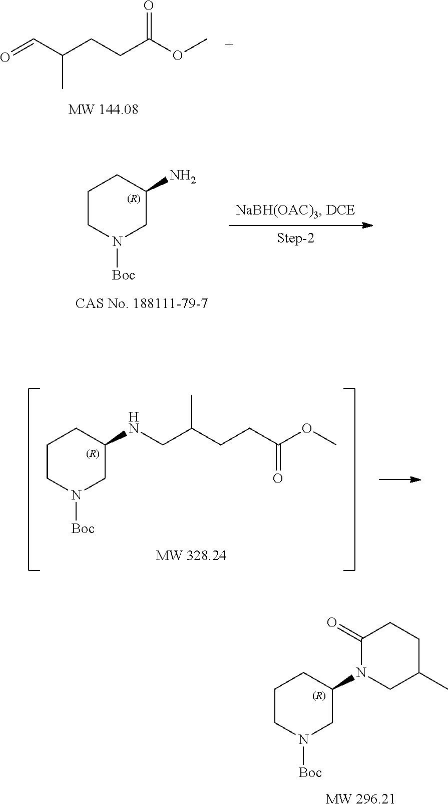

In an oven dried single neck round bottom flask was charged solution of methyl 4-methyl-5-oxopentanoate (2.0 g, 0.0138 mol) and CAS No. 188111-79-7 (2.7 g, 0.0138 mol) in dichloroethane (10 ml) and stirred for 10 min. Acetic acid (2 ml) was charged to reaction mixture and allowed to stir at room temperature for 30 min. sodium-triacetoxyborohydride (4.4 g, 0.020 mol) was charged to reaction mixture at room temperature and stirred at room temperature for 12 h. The reaction mixture was diluted with water (100 mL) and extracted in (250 mL×2) ethyl acetate. The combined organic layer was dried over anhydrous sodium sulphate, filtered and concentrated under reduce pressure. Crude material was then purified by column chromatography using 10% EA: Hexane to afford 1.0 g as colourless sticky solid. LCMS purity 92.20% (Method A).

### Step-3: Synthesis of (3′R)-5-methyl-[1, 3′-bipiperidin]-2-one

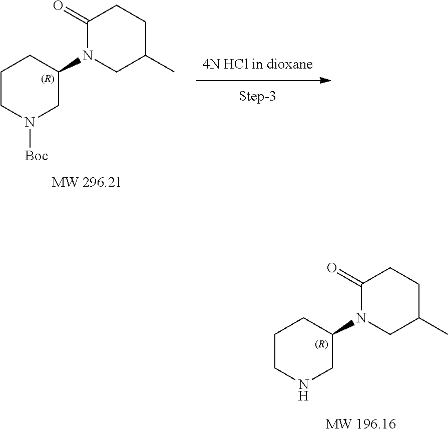

In an oven dried single neck round bottom flask was added tert-butyl (3′R)-5-methyl-2-oxo-[1,3′-bipiperidine]-1′-carboxylate (1.0 g, 0.0012 mol) in DCM (4 ml) and stirred for 5 min. 1N HCl in dioxane (4 ml) was charged to and resulting reaction mixture was stirred at room temperature for 2 h. The reaction mixture was directly evaporated under reduced pressure to afford 1.0 g white solid as desired product. LCMS purity 75.00% (Method A).

### Step-4: Synthesis of 4-nitrophenyl (3′R)-5-methyl-2-oxo-[1,3′-bipiperidine]-1′-carboxylate

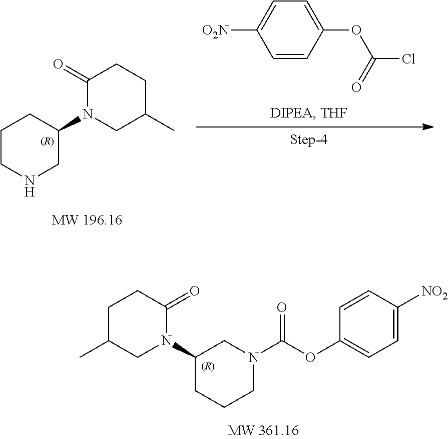

In an oven dried single neck round bottom flask was added (3′R)-5-methyl-[1, 3′-bipiperidin]-2-one (0.500 g, 0.0025 mol) in THF (5 ml) and stirred for 5 min followed by addition of DIPEA (1.3 ml, 0.0076 mol) at room temperature. The resulting reaction mixture was stirred for 10 min and 4-nitrophenyl chloroformate (0.512 g, 0.0025 mol) was added and allowed to stir at room temperature for 30 min. Reaction mixture was diluted with water (50 mL) and extracted with ethyl acetate (100 mL×2). The combined organic layer was dried over sodium sulfate, filtered and concentrated under reduced pressure. Crude material was then purified by column chromatography using 1% methanol in DCM to afford 0.25 g 4-nitrophenyl (3′R)-5-methyl-2-oxo-[1,3′-bipiperidine]-1′-carboxylate as colourless sticky solid. LCMS purity 98.72% (Method A).

### Step-5: Synthesis of (3′R)—N—((S)-3-(3,4-dihydroisoquinolin-2(1H)-yl)-2-hydroxypropyl)-5-methyl-2-oxo-[1,3′-bipiperidine]-1′-carboxamide

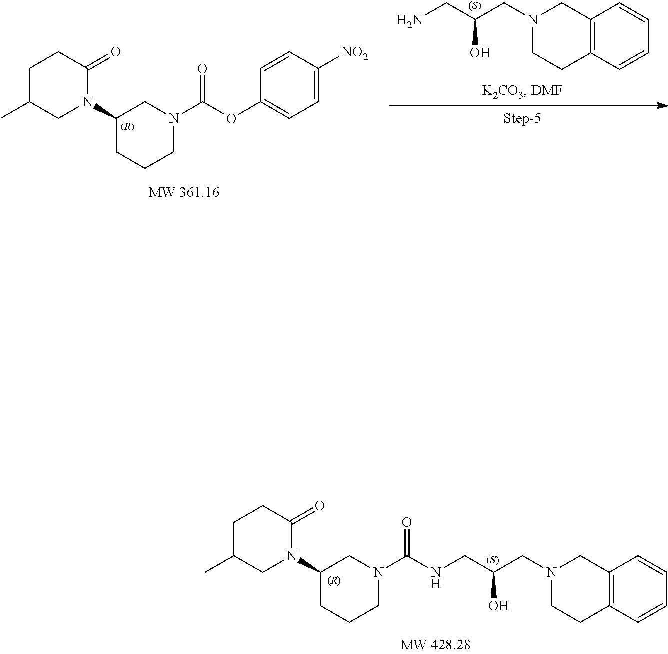

To a stirred solution of 4-nitrophenyl (3′R)-5-methyl-2-oxo-[1,3′-bipiperidine]-1′-carboxylate (0.250 g, 0.00069 mol) in DMF (3.0 mL) was added (S)-1-amino-3-(3,4-dihydroisoquinolin-2(1H)-yl)propan-2-ol (Int-A3) shown above (0.213 g, 0.0010 mol) and K₂CO₃ (0.382 g, 0.00027 mol) at room temperature. Reaction mixture was then heated at 65° C. for 48-50 h. The reaction mixture was diluted with ice cold water (100 mL) and extracted with ethyl acetate (200 ml×2). Combined organic layer was washed with brine (50 mL×2). Organic layer was dried over anhydrous sodium sulphate, filtered and concentrated under reduced pressure. The obtained crude was purified by reverse phase flash chromatography using 0.1% ammonia solution in water:acetonitrile to afford (0.15 g, Yield 50.60%) (3′R)—N—((S)-3-(3,4-dihydroisoquinolin-2(1H)-yl)-2-hydroxypropyl)-5-methyl-2-oxo-[1,3′-bipiperidine]-1′-carboxamide as a yellow sticky solid. LCMS purity 98.10% (Method A). 1H NMR: 400 MHz, DMSO-de: 7.099-7.023 (4H, m), 6.58-6.57 (t, 1H), 4.147-4.119 (m, 1H), 3.823-3.759 (m, 4H), 3.662-3.608 (m, 3H), 3.279-3.216 (m, 2H), 3.014-2.982 (m, 1H), 2.800-2.684 (m, 5H), 2.43-2.402 (m, 3H), 2.262-2.245 (m, 2H), 1.693 (m, 2H), 1.609-1.591 (d, 3H), 1.346-1.305 (m, 3H), 0.948-0.935 (d, 3H).

Using the chiral HPLC method, the following characterisation data were obtained for specific stereoisomers.

| | |
|---|---|
| (3′R,5S)-N-((S)-3-(3,4-dihydroisoquinolin- 2(1H)-yl)-2-hydroxypropyl)-5-methyl-2- oxo-[1,3′-bipiperidine]-1 -carboxamide | 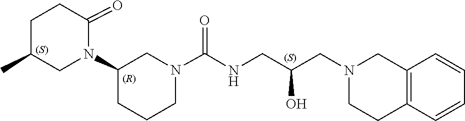 |
| LCMS (Method A): Peak-1, 1.156 min | 1H NMR: 400 MHz, DMSO-d₆: 7.085-7.031 (4H, |
| MS: ES+ 429.6 (M + 1) | m), 6.591 (t, 1H), 4.865 (s, 1H), 4.135 (t, 1H), |
| | 3.824-3.732 (m, 3H), 3.608-3.594 (m, 2H), |
| | 3.214-3.148 (m,2H), 3.008-2.992 (m, 1H), 2.794- |
| | 2.653(m,6H), 2.324(m, 2H), 2.260-2.248 (m, |
| | 2H), 1.792 (s,1H) 1.689 (s, 1H), 1.606-1.589 (s, |
| | 3H), 1.348-1.227(m, 3H), 0.945-0.929(d, 3H) |
| (3′R,5R)-N-((S)-3-(3,4- dihydroisoquinolin-2(1H)-yl)-2- hydroxypropyl)-5-methyl-2-oxo-[1,3′- bipiperidine]-1′-carboxamide | 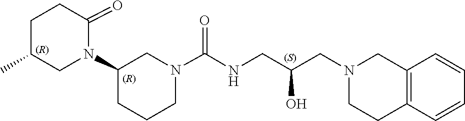 |
| LCMS (Method A): Peak-2, 1.157 min | 1H NMR: 400 MHz, DMSO-d₆: 7.106-7.054 (4H, |
| MS: ES+ 429.6 (M + 1) | m), 6.646 (s, 1H), 4.941-4.896 (s, 1H), 4.116 (t, |
| | 1H), 3.838-3.809 (d, 3H), 3.738 (s, 1H), 3.485- |
| | 3.471 (m,1H), 3.418-3.404 (m, 1H), 3.279- |
| | 3.261(d,1H), 3.179-3.148(m, 1H), 3.023-3.007 |
| | (m, 1H), 2.826(s,3H) 2.740-2.683 (t, 3H), 2.259- |
| | 2.245 (s, 2H) 1.793(s, 1H) 1.714-1.690(s, |
| | 1H), 1.648-1.562(m, 3H) 1.360-1.331(m, 3H) |
| | 0.951-0.935(d, 3H) |

### Example 4

### Preparation of (R)—N—(S)-3-(3,4-dihydroisoquinolin-2(1H)-yl-1,1-d2)-2-hydroxypropyl)-2-oxo-[1,3′-bipiperidine]-1′-carboxamide

The synthetic steps shown in the reaction scheme below were performed.

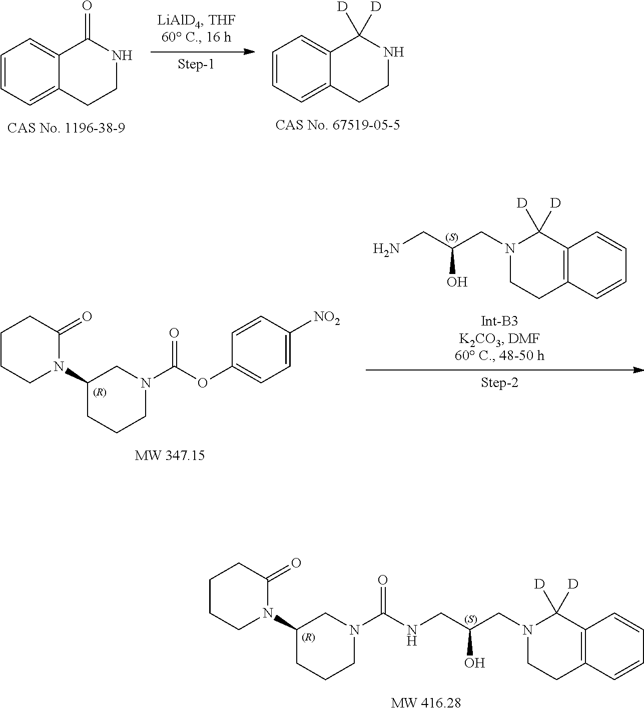

### Step-1: Synthesis of 1,2,3,4-tetrahydroisoquinoline-1,1-d2

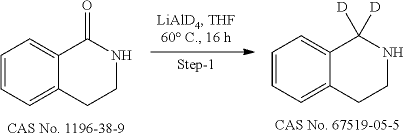 [SMILES: O=C1NCCc2ccccc21 ; "LiAlD4, THF60° C., 16 h" ; "Step-1" ; [2H]C1([2H])NCCc2ccccc21]

In an oven dried single neck round bottom flask was charged a solution of CAS No. 1196-38-9 (1.0 g, 0.0068 mol) in THF (10 v, 10 mL) and reaction mixture was cooled to −40° C. LiAlD₄ (0.430 g, 0.0102 mol) was added slowly portion wise. The reaction mixture was allowed to stir at 70° C. for 16 h. The reaction mixture was quenched with 10% w/w NaOH solution at 0° C. (100 mL) and extracted with ethyl acetate (100 mL×3). The combined organic layer was dried over anhydrous sodium sulphate, filtered and concentrated under reduce pressure to afford 0.520 g of 1,2,3,4-tetrahydroisoquinoline-1,1-d2 (0.520 g, Yield: 56.66%.) as brown solid. LCMS purity 100% (Method A).

### Step-2 Synthesis of (R)—N—((S)-3-(3,4-dihydroisoquinolin-2(1H)-yl-1,1-d2)-2-hydroxypropyl)-2-oxo-[1,3′-bipiperidine]-1′-carboxamide

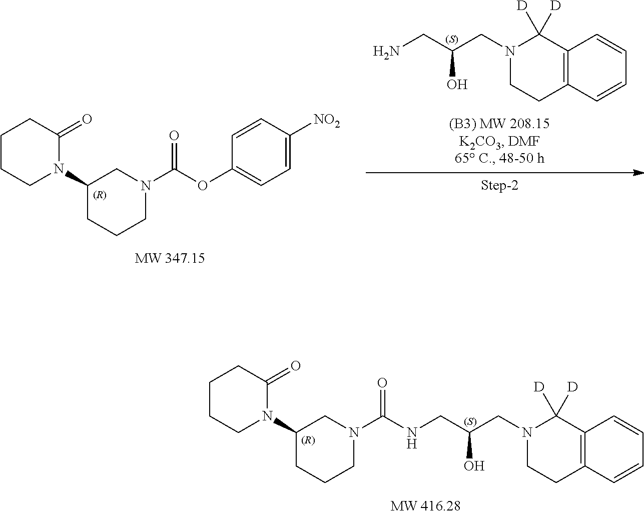

4-nitrophenyl (3′R)-2-oxo-[1,3′-bipiperidine]-1′-carboxylate was prepared as described in step-4 of Example 2. To a stirred solution of 4-nitrophenyl (3′R)-2-oxo-[1,3′-bipiperidine]-1′-carboxylate (0.058 g, 0.00016 mol) in DMF (2.0 mL) was added Intermediate (B3) (0.052 g, 0.00025 mol) and potassium carbonate (0.092 g, 0.00064 mol) at room temperature. Intermediate (B3) shown above was prepared from 1,2,3,4-tetrahydroisoquinoline-1,1-d2 using the method described above for preparing (S)-1-amino-3-(3,4-dihydroisoquinolin-2(1H)-yl)propan-2-ol (Int-A3). Reaction mixture was then heated at 65° C. for 48-50 h. The reaction mixture was diluted with ice cold water (100 mL) and extracted with ethyl acetate (200 mL×2). Combined organic layer was washed with brine (20 ml×2). Organic layer was dried over anhydrous sodium sulphate, filtered and concentrated under reduced pressure. The obtained crude was purified by reverse phase flash chromatography using 0.1% ammonia solution in water:acetonitrile to afford (R)—N—((S)-3-(3,4-dihydroisoquinolin-2(1H)-yl-1, 1-d2)-2-hydroxypropyl)-2-oxo-[1,3′-bipiperidine]-1′-carboxamide (0.03 g, Yield: 28.76%). LCMS purity 98.64% (Method A). 1H NMR: 400 MHZ, DMSO-d₆: 7.10-7.02 (4H, m), 6.579 (t, 1H), 4.84 (d, J=4 Hz, 1H), 4.15-4.10 (m, 1H), 3.79-3.776 (m, 3H), 3.20-3.15 (m, 3H), 3.11-3.10 (m, 2H), 3.00-2.97 (m, 2H), 2.791-2.674 (m, 3H), 2.23-2.20 (m, 2H), 1.659-1.578 (m, 7H), 1.377-1.325 (m, 1H).

### Example 5

### Preparation of (R)—N—(S)-3-(3,4-dihydroisoquinolin-2(1H)-yl-1,1,4,4-d4)-2-hydroxypropyl)-2-oxo-[1,3′-bipiperidine]-1′-carboxamide

The synthetic steps shown in the reaction scheme below were performed.

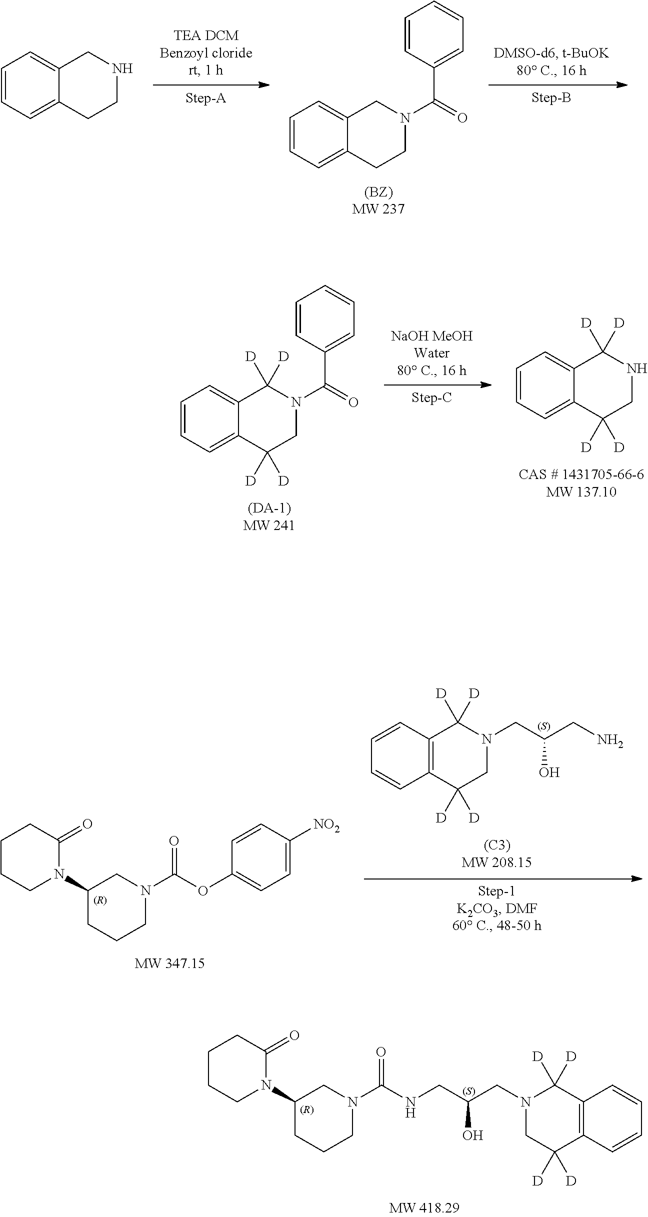

### Step-A: Synthesis of (3, 4-dihydroisoquinolin-2(1H)-yl) (phenyl)methanone

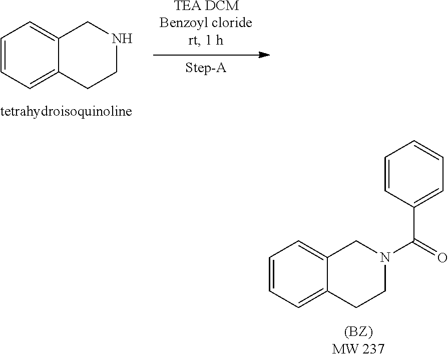 [SMILES: c1ccc2c(c1)CCNC2 ; "Step-A" ; "TEA DCMBenzoyl cloridert, 1 h" ; O=C(c1ccccc1)N1CCc2ccccc2C1]

In an oven dried single neck round bottom flask was added tetrahydroisoquinoline (3.0 g, 0.022 mol) in DCM (30 ml, 10 v) and cooled to 0° C. and TEA was added. After 15 min benzoyl chloride (3.9 ml, 0.033 mol) was added to it and stirred the solution for 1 h at room temperature. The progress of reaction was monitored using TLC (50% ethyl acetate:hexane). After completion, reaction mixture was added water (100 mL) and extracted in DCM (100 ml×2). Combined organic layer was then dried over anhydrous sodium sulphate, filtered and concentrated. Crude material was then purified using 15% EA: Hexane to afford Intermediate (BZ) (4.8 g, Yield 89.81%) as yellow sticky oil. LCMS purity 89.81% (Method A).

### Step-B: Synthesis of (3,4-dihydroisoquinolin-2(1H)-yl-1,1,4,4-d4)(phenyl)methanone

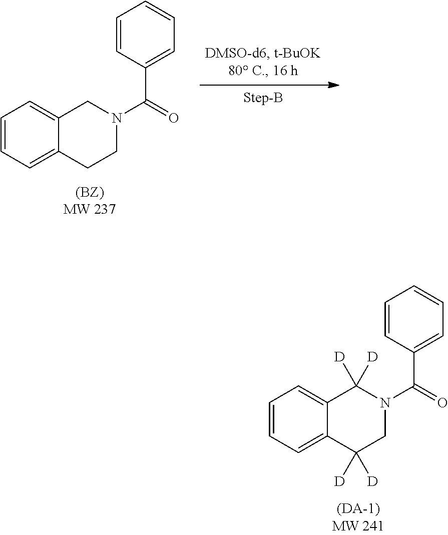 [SMILES: O=C(c1ccccc1)N1CCc2ccccc2C1 ; "DMSO-d6, t-BuOK80° C., 16 h" ; "Step-B" ; [2H]C1([2H])CN(C(=O)c2ccccc2)C([2H])([2H])c2ccccc21]

Potassium tert-butoxide (0.070 mg, 0.0063 mol) was added to a previously stirred solution of (3, 4-dihydroisoquinolin-2(1H)-yl) (phenyl)methanone (Intermediate (BZ)) (0.500 g, 0.022 mol) in DMSO-d6 (15 ml, 3 v) and the resulting reaction mixture was heated at 80° C. for 16 h. The progress of reaction was monitored using LCMS. (Another 4 batches using above stoichiometry was performed and combined during workup). After completion, reaction mixture was added water (3000 mL) and extracted in ethyl acetate (400 ml×2), The combined organic layer was then dried over anhydrous sodium sulphate, filtered and concentrated under reduced pressure. Crude material was then purified using 15% EA:hexane to afford (3,4-dihydroisoquinolin-2(1H)-yl-1,1,4,4-d4)(phenyl)methanone (Intermediate (DA-1)) (0.75 g, Yield 73.75%) as a yellow sticky solid. LCMS purity 99.10% (Method A).

### Step-C: Synthesis of 1,2,3,4-tetrahydroisoquinoline-1,1,4,4-d4

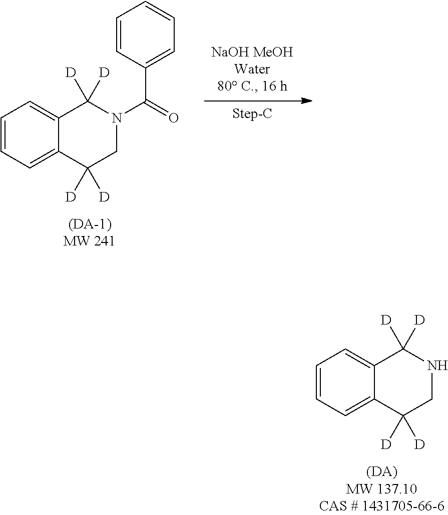 [SMILES: [2H]C1([2H])CN(C(=O)c2ccccc2)C([2H])([2H])c2ccccc21 ; "NaOH MeOHWater80° C., 16 h" ; "Step-C" ; [2H]C1([2H])CNC([2H])([2H])c2ccccc21]

To a stirred solution of (3,4-dihydroisoquinolin-2(1H)-yl-1, 1,4,4-d4)(phenyl)methanone (Intermediate (DA-1)) (1.1 g, 0.0045 mol) in methanol and water (8 mL+4 mL) was added NaOH (3.6 g, 0.091 mol) and reaction mixture was heated at 80° ° C. for 16 h. The progress of reaction was monitored using of TLC (10% methanol in DCM). After completion, reaction mixture was added water (100 mL) and extracted in ethyl acetate (200 mL×2), combined organic layer was then dried over anhydrous sodium sulphate, filtered and concentrated under reduced pressure. Crude material was then purified using 3% MeOH: DCM to afford 1,2,3,4-tetrahydroisoquinoline-1,1,4,4-d4 (Intermediate (DA)) (0.850 g, Yield 73.28%) as yellow solid. LCMS purity 84.85% (Method A).

### Step-1: Synthesis of (R)—N—((S)-3-(3,4-dihydroisoquinolin-2(1H)-yl-1,1,4,4-d4)-2-hydroxypropyl)-2-oxo-[1,3′-bipiperidine]-1′-carboxamide

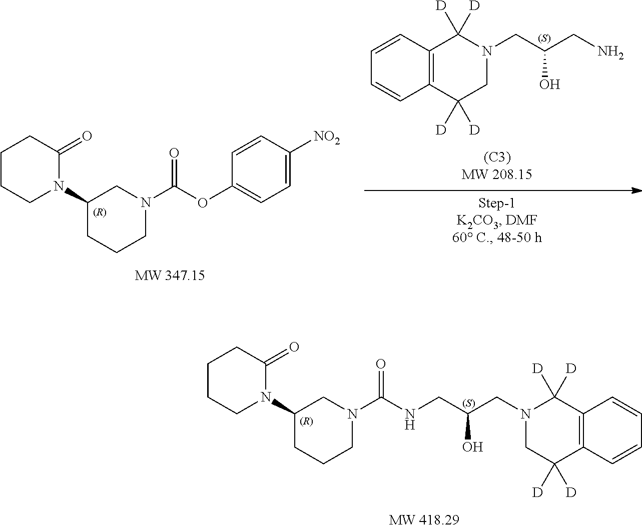

4-nitrophenyl (3′R)-2-oxo-[1,3′-bipiperidine]-1′-carboxylate was prepared as described in step-4 of Example 2. To a stirred solution of 4-nitrophenyl (3′R)-2-oxo-[1,3′-bipiperidine]-1′-carboxylate (0.80 g, 0.00023 mol) in DMF (3 mL) was added Intermediate (C₃) (0.072 g, 0.00034 mol) and K₂CO₃ (0.127 g, 0.00092 mol) and the reaction mixture was heated at 65° C. for 48 h. Intermediate (C₃) shown above was prepared from 1,2,3,4-tetrahydroisoquinoline-1,1,4,4-d4 using the method described above for preparing (S)-1-amino-3-(3,4-dihydroisoquinolin-2(1H)-yl)propan-2-ol (Int-A3). The progress of reaction was monitored using TLC (5% Methanol: DCM+ ammonia). RM was then diluted with water (50 mL) and extracted in ethyl acetate (100 mL×2). The combined organic layer dried over anhydrous sodium sulfate, filtered and concentrated. Crude material was then purified using reverse-phase column chromatography using 0.1% ammonia in water: acetonitrile to afford (R)—N—((S)-3-(3,4-dihydroisoquinolin-2(1H)-yl-1,1,4,4-d4)-2-hydroxypropyl)-2-oxo-[1,3′-bipiperidine]-1′-carboxamide (39 mg, 40.46%) as the desired product. 1H NMR: 400 MHZ, DMSO-d₆: 7.104-7.026 (4H, m), 6.658 (br s, 1H), 4.844 (br s, 1H), 4.157-4.103 (m, 1H), 3.793-3.777 (m, 3H), 3.210-3.114 (m, 3H), 3.040 (m, 1H), 3.737-2.677 (t, 4H), 2.499 (m, 2H), 2.238-2.207 (t, 2H), 1.661-1.591 (m, 7H), 1.358-1.326 (m, 1H).

### Example 6

### Preparation of (3′R)—N—((S)-3-(3,4-dihydroisoquinolin-2(1H)-yl)-2-hydroxypropyl)-3-methyl-2-oxo-[1,3′-bipiperidine]-1′-carboxamide

The synthetic steps shown in the reaction scheme below were performed.

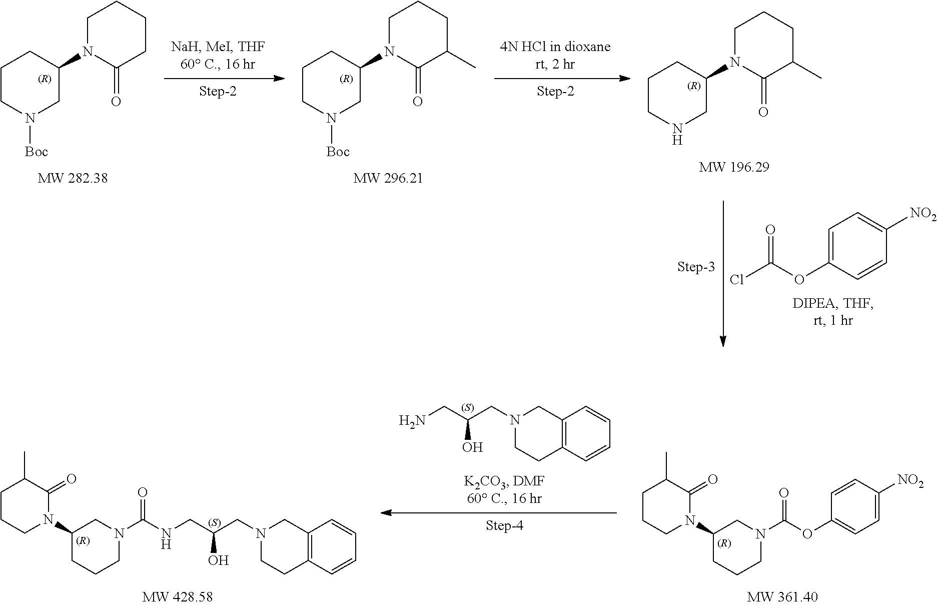

### Step-1: Synthesis of tert-butyl (3′R)-3-methyl-2-oxo-[1,3′-bipiperidine]-1′-carboxylate

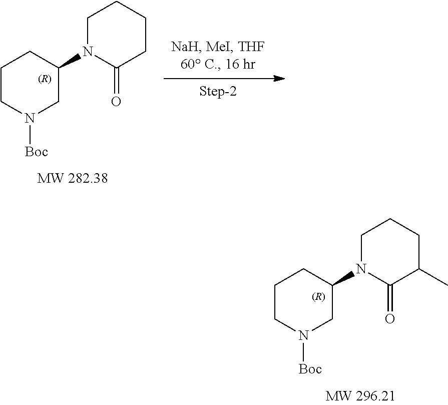

The intermediate tert-butyl (3′R)-2-oxo-[1,3′-bipiperidine]-1′-carboxylate was prepared using the method in Step 2 of Example 2. In an oven dried single neck round bottom flask was charged a solution of tert-butyl (3′R)-2-oxo-[1,3′-bipiperidine]-1′-carboxylate (5 g, 0.0177 mol) in THF (6 v, 30 mL) and reaction mixture was cooled to 0° C. and NaH (1.41 g, 0.0354 mol) was added. After 1 h of stirring at 70° ° C. the reaction mixture was cooled to 0° C. and methyl Iodide (3.75 g, 0.0265 mol) was added dropwise. Reaction mixture was stirred overnight at 70° C. The reaction mixture was diluted with ice cold water (100 mL) and extracted with ethyl acetate (100 mL×2). The combined organic layer was dried over anhydrous sodium sulphate, filtered and concentrated under reduce pressure to produce a brown coloured oil. The resulting crude material was purified by column chromatography using 50% ethyl acetate in hexane to afford tert-butyl (3′R)-3-methyl-2-oxo-[1,3′-bipiperidine]-1′-carboxylate (0.2 g, Yield 3.81%) as a colourless oil. LCMS purity 89.36% (Method A).

### Step-2: Synthesis of (3′R)-3-methyl-[1,3′-bipiperidin]-2-one

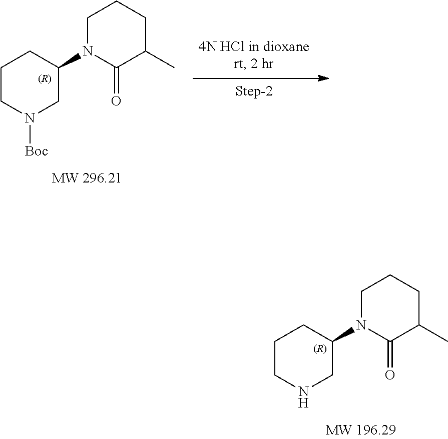

To a stirred solution of tert-butyl (3′R)-3-methyl-2-oxo-[1,3′-bipiperidine]-1′-carboxylate (0.2 g, 0.675 mol) in DCM (3 mL) was added 4N HCl in dioxane (3 mL) at room temperature. The reaction mixture was allowed to stir at room temperature for 2 h. Reaction mixture was directly concentrated under reduce pressure to afford (3′R)-3-methyl-[1,3′-bipiperidin]-2-one (0.2 g, Yield Qnt.) as a white sticky solid. Isolated solid was used for next step without further purification. LCMS purity 58.56% (Method A).

### Step-3: Synthesis of 4-nitrophenyl (3′R)-3-methyl-2-oxo-[1,3′-bipiperidine]-1′-carboxylate

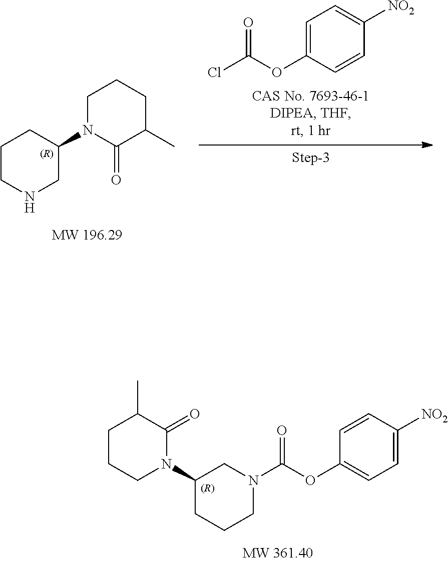

To a stirred solution of (3′R)-3-methyl-[1,3′-bipiperidin]-2-one (0.2 g, 0.0010 mol) in THF (5 mL) was added DIPEA (0.394 g, 0.0030 mol) and stirred for 15 min at room temperature followed by 4-nitrophenyl chloroformate (0.205 g, 0.0010 mol). The reaction mixture was further stirred at room temperature for 1 h. Reaction mixture was diluted with water (100 mL) and extracted with ethyl acetate (100 mL×2). The combined organic layer was dried over anhydrous sodium sulphate, filtered and concentrated under reduced pressure. The obtained crude was purified by column chromatography using 50% ethyl acetate in hexane to afford 4-nitrophenyl (3′R)-3-methyl-2-oxo-[1,3′-bipiperidine]-1′-carboxylate (0.150 g, Yield: 40.74%) as sticky white solid. LCMS purity 92.01% (Method A).

### Step-4: Synthesis of (3′R)—N—((S)-3-(3,4-dihydroisoquinolin-2(1H)-yl)-2-hydroxypropyl)-3-methyl-2-oxo-[1,3′-bipiperidine]-1′-carboxamide

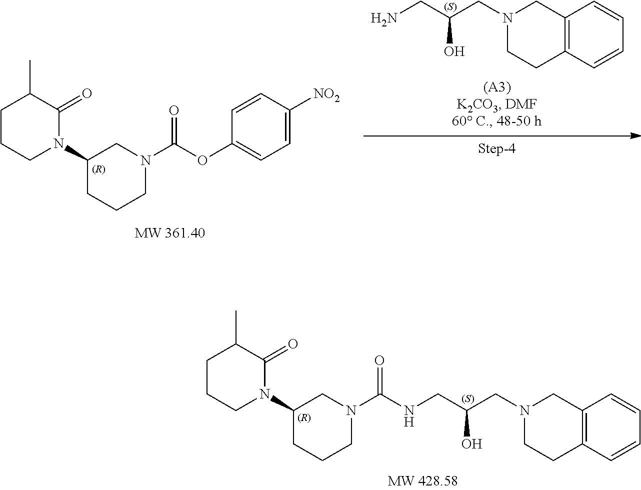

To a stirred solution of 4-nitrophenyl (3′R)-3-methyl-2-oxo-[1,3′-bipiperidine]-1′-carboxylate (0.150 g, 0.00041 mol) in DMF (3 mL) were added (S)-1-amino-3-(3,4-dihydroisoquinolin-2(1H)-yl)propan-2-ol (Int-A3) (0.128 g, 0.000615 mol) and potassium carbonate (0.262 g, 0.00164 mol) at room temperature. The reaction mixture was heated at 65° C. for 48-50 h. The reaction progress was monitored using TLC (Mobile Phase: 5% MeOH in DCM under NH₃ atmosphere). After consumption of 4-nitrophenyl (3′R)-3-methyl-2-oxo-[1,3′-bipiperidine]-1′-carboxylate, the reaction mixture was diluted with ice cold water (100 mL) and extracted with ethyl acetate (100 mL×2). Combined organic layer was washed with brine (50 mL×3). Organic layer was dried over anhydrous sodium sulphate, filtered and concentrated under reduced pressure. The obtained crude was purified by reverse phase flash chromatography using 0.1% ammonia solution in water:acetonitrile to afford of (3′R)—N—((S)-3-(3,4-dihydroisoquinolin-2(1H)-yl)-2-hydroxypropyl)-3-methyl-2-oxo-[1,3′-bipiperidine]-1′-carboxamide as yellow sticky solid. (0.065 g, Yield: 36.54%). LCMS purity 99.19% (Method A). 1H NMR: 400 MHZ, DMSO-d₆: 7.09-7.03 (4H, m), 6.58-6.56 (m, 1H), 4.83-4.82 (br s, 1H), 4.16-4.10 (m, 1H), 3.816-3.775 (m, 3H), 3.60-3.58 (q, 2H), 3.27-3.16 (m, 3H), 3.01 (m, 1H), 2.791-2.64 (m, 7H), 2.42-2.24 (m, 2H), 1.86-1.73 (m, 2H), 1.59-1.58 (m, 4H), 1.352-1.320 (m, 2H), 1.07-1.09 (m, 3H).

### Example 7

### Preparation of (R)—N—((S)-3-(3,4-dihydroisoquinolin-2(1H)-yl)-2-hydroxypropyl)-3-(5-methyl-6-oxopyridazin-1(6H)-yl)piperidine-1-carboxamide

The synthetic steps shown in the reaction scheme below were performed.

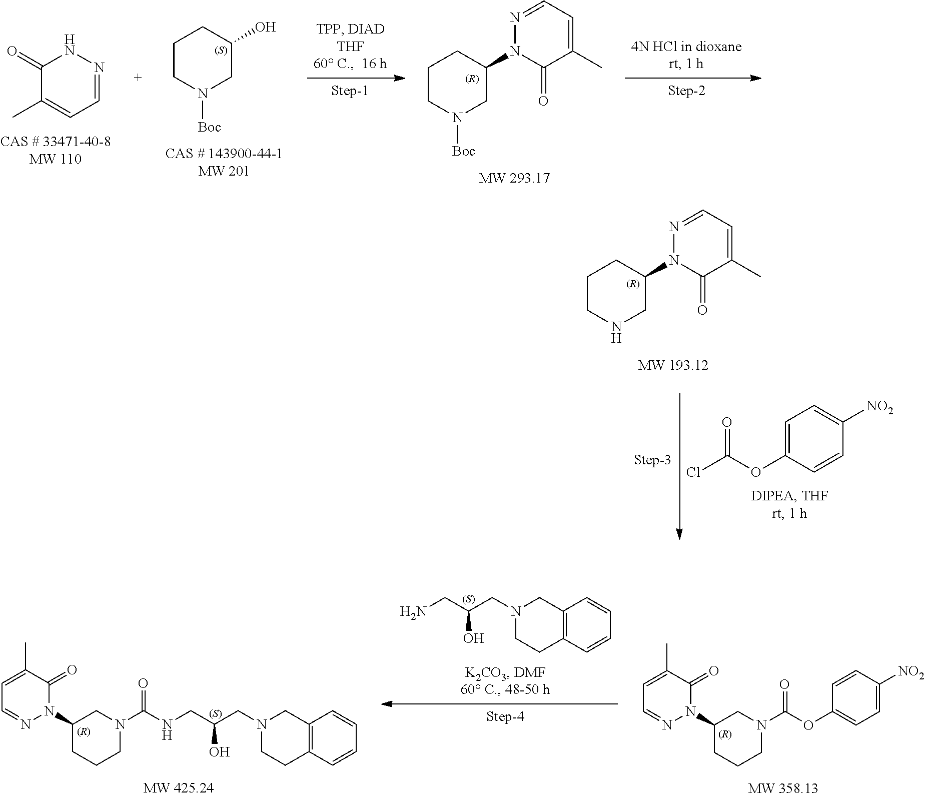

### Step-1: Synthesis of tert-butyl (R)-3-(5-methyl-6-oxopyridazin-1(6H)-yl)piperidine-1-carboxylate

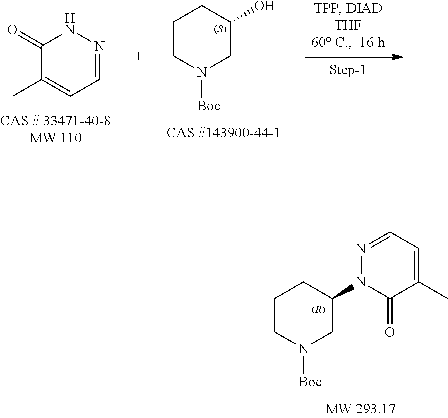

In an oven dried single neck round bottom flask was charged a solution of CAS No. 33471-40-8 (0.154 g, 0.00099 mol) and CAS No. 143900-44-41 (0.200 g, 0.00099 mol) in dry THF (5 mL) and TPP (0.520 g, 0.00198 mol) was added. The reaction mixture was allowed to stir at 70° C. for 30 min, after that DIAD (0.734 g, 0.00198 mol) was added dropwise over period of 10 min. The reaction mixture was allowed to stir at 70° C. for 16 h. Reaction progress was monitored using TLC (Mobile Phase: neat ethyl acetate). The reaction mixture was diluted with water (30 mL) and extracted with ethylacetate (30 ml×2). The combined organic layer was dried over anhydrous sodium sulphate, filtered and concentrated under reduce pressure to afford 0.5 g (crude). The obtained crude was purified by silica gel (60-120 mesh) column chromatography using 20% ethylacetate in hexane to afford tert-butyl (R)-3-(5-methyl-6-oxopyridazin-1(6H)-yl)piperidine-1-carboxylate (0.311 g, Yield 39.00%) as a colourless liquid. LCMS purity 96.71% (Method A).

### Step-2: Synthesis of (R)-4-methyl-2-(piperidin-3-yl)pyridazin-3(2H)-one

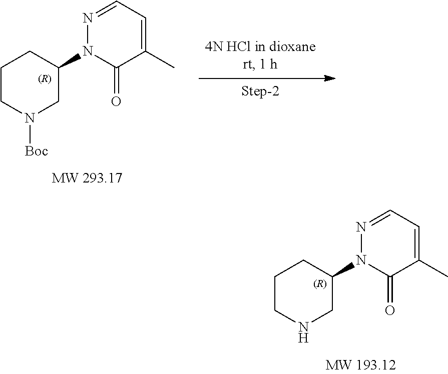

To a stirred solution of tert-butyl (R)-3-(5-methyl-6-oxopyridazin-1(6H)-yl)piperidine-1-carboxylate (0.311 g, 0.00106 mol) in DCM (5 mL) was added 4N HCl in dioxane (3 mL) at room temperature. The reaction mixture was allowed to stir at room temperature for 2 h. Reaction mixture was directly concentrated under reduce pressure to afford (R)-4-methyl-2-(piperidin-3-yl)pyridazin-3(2H)-one (0.280 g, Yield: Qnt.) as a white sticky solid. Crude material was directly used for next step without further purification. LCMS purity 88.71% (Method A).

### Step-3: Synthesis of 4-nitrophenyl (R)-3-(5-methyl-6-oxopyridazin-1(6H)-yl)piperidine-1-carboxylate

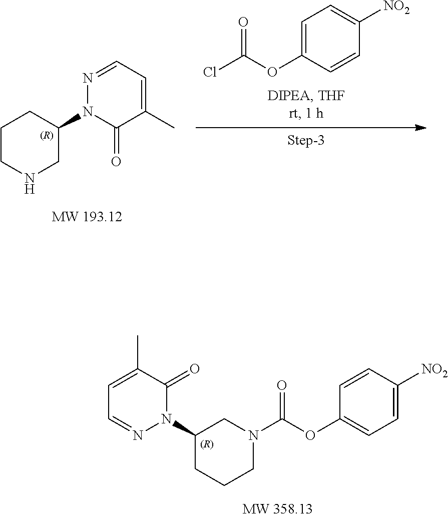

To a stirred solution of (R)-4-methyl-2-(piperidin-3-yl)pyridazin-3(2H)-one (0.280 g, 0.0014 mol) in THF (5 mL) was added DIPEA (0.561 g, 0.0043 mol) at room temperature and stirred for 15 min followed by addition of 4-nitrophenyl chloroformate (0.291 g, 0.0014 mol). The reaction mixture was allowed to stir at room temperature for 1 h. Reaction mixture was diluted with water (50 mL) and extracted with ethyl acetate (50 mL×2). The combined organic layer was dried over anhydrous sodium sulphate, filtered and concentrated under reduced pressure. The obtained crude was purified by silica gel (60-120 mesh) column chromatography using 50% ethylacetate in hexane to afford 4-nitrophenyl (R)-2-oxo-[1,3′-bipiperidine]-1′-carboxylate (0.169 g, Yield 32.25%) as a white sticky solid. LCMS purity 97.35% (Method A).

### Step-4: Synthesis of (R)—N—((S)-3-(3,4-dihydroisoquinolin-2(1H)-yl)-2-hydroxypropyl)-3-(5-methyl-6-oxopyridazin-1(6H)-yl)piperidine-1-carboxamide

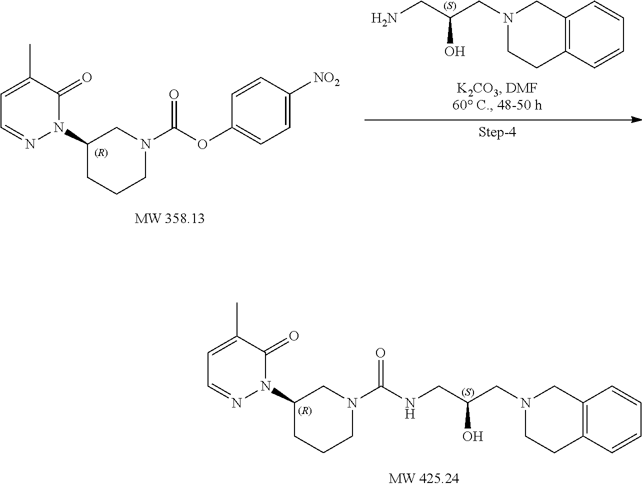

To a stirred solution of 4-nitrophenyl (R)-2-oxo-[1,3′-bipiperidine]-1′-carboxylate (0.169 g, 0.0047 mol) in DMF (3.0 mL) was added (S)-1-amino-3-(3,4-dihydroisoquinolin-2(1H)-yl)propan-2-ol (Int-A3) (0.145 g, 0.0070 mol) and potassium carbonate (0.266 g, 0.0188 mol) at room temperature. The reaction mixture was heated at 65° C. for 48-50 h. The reaction progress was monitored using TLC (Mobile Phase: 5% MeOH in DCM under NH₃ atmosphere). After consumption of Int-A3), the reaction mixture was diluted with ice cold water (100 mL) and extracted with ethyl acetate (100 mL×2). Combined organic layer was washed with chilled brine (100 mL×3). Organic layer was dried over anhydrous sodium sulphate, filtered and concentrated under reduced pressure. The obtained crude was purified by reverse phase flash chromatography using 0.1% ammonia solution in water:acetonitrile to afford (R)—N—((S)-3-(3,4-dihydroisoquinolin-2(1H)-yl)-2-hydroxypropyl)-3-(5-methyl-6-oxopyridazin-1(6H)-yl)piperidine-1-carboxamide (0.05 g, Yield 14.95%). LCMS purity 100% (Method A). 1H NMR: 400 MHZ, DMSO-d6: 7.86-7.85 (1H, d), 7.30-7.17 (5H, m), 6.89-6.87 (t, 1H), 5.85 (s, 1H), 4.73-4.70 (m, 1H), 4.60-4.53 (m, 1H), 4.43-4.33 (m, 1H), 4.09-3.98 (m, 3H), 3.79-3.70 (m, 1H), 3.28-3.30 (m, 1H), 3.21-3.19 (m, 3H), 3.03-3.15 (m, 1H), 2.70-3.67 (m, 1H), 2.10-2.09 (s, 3H), 1.86-1.72 (m, 3H), 1.505 (m, 1H), 1.299-1.23 (m, 2H), 0.87-0.83 (t, 1H).

### Example 8

### Preparation of (R)—N—((S)-3-(3,4-dihydroisoquinolin-2(1H)-yl)-2-hydroxypropyl)-3-(3-methyl-2-oxotetrahydropyrimidin-1(2H)-yl)piperidine-1-carboxamide

The synthetic steps shown in the reaction scheme below were performed.

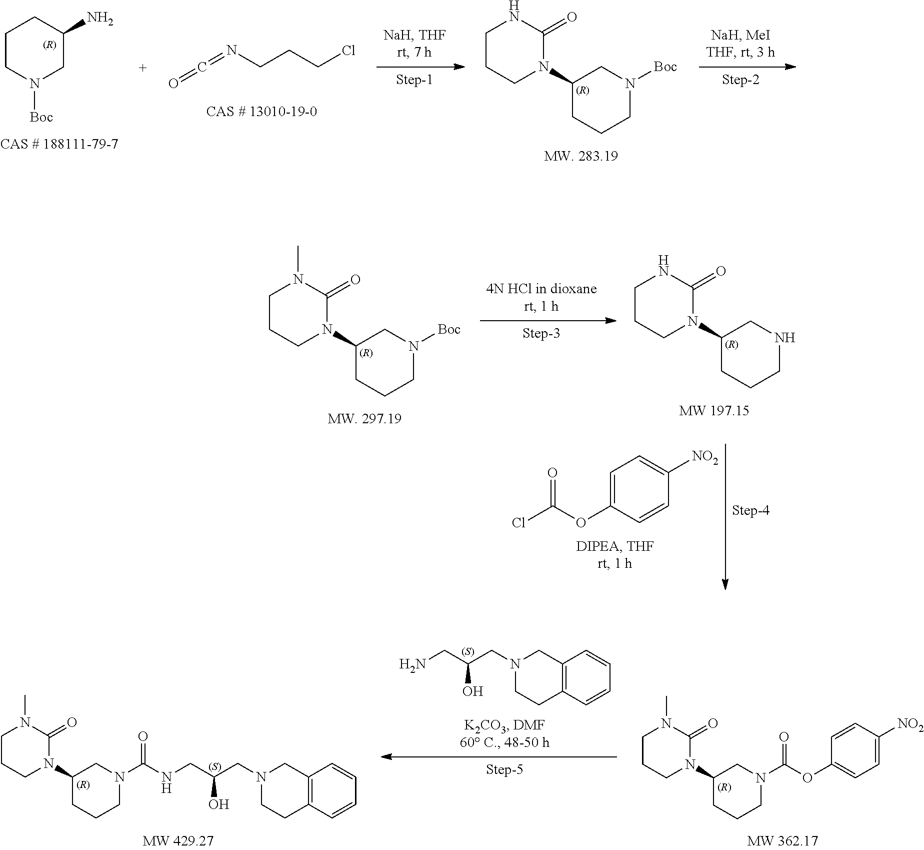

### Step-1: Synthesis of tert-butyl (R)-3-(2-oxotetrahydropyrimidin-1(2H)-yl)piperidine-1-carboxylate

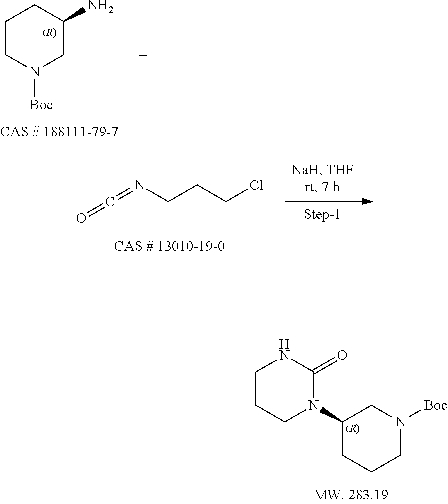

In an oven dried single neck round bottom flask was charged a solution of CAS No. 188111-79-7 (1.0 g, 0.005 mol) in THF (10 ml, 10 v) and CAS No. 13010-19-0 (0.597 g, 0.005 mol) was added in to the reaction mixture. The reaction mixture was allowed to stir at room temperature for 3 h and 60% NaH in mineral (0.288 g, 0.012 mol) was charged portion-wise over period of 15 min. The reaction mixture was allowed to stir at room temperature for further 4 h. The reaction mixture was diluted with cold water (60 mL) and extracted with ethyl acetate (60 mL×2). The combined organic layer was dried over anhydrous sodium sulphate, filtered and concentrated under reduce pressure to afford tert-butyl (R)-3-(2-oxotetrahydropyrimidin-1(2H)-yl) piperidine-1-carboxylate (1.5 g, Yield Qnt.) as a white solid. LCMS purity 100% (Method A).

### Step-2: Synthesis of tert-butyl (R)-3-(3-methyl-2-oxotetrahydropyrimidin-1(2H)-yl)piperidine-1-carboxylate

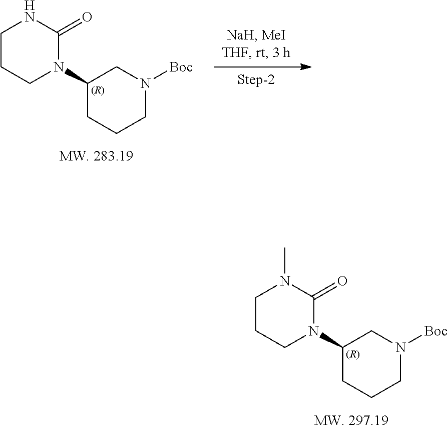

To a stirred solution of tert-butyl (R)-3-(2-oxotetrahydropyrimidin-1(2H)-yl) piperidine-1-carboxylate (0.5 g, 0.0017 mol) in DMF (5 mL) was added 60% NaH in mineral (0.706 g, 0.017 mol) followed by addition of Mel (0.11 mL, 0.017 mol) at room temperature. The reaction mixture was allowed to stir at room temperature for 3 h. The reaction mixture was quenched with MeOH (˜10 mL) and diluted with water (200 mL). Aqueous layer was then extracted with ethyl acetate (100 mL×2). The combined organic layer was washed with chilled brine solution (200 mL), dried over anhydrous sodium sulphate, filtered and concentrated under reduce pressure to afford tert-butyl (R)-3-(3-methyl-2-oxotetrahydropyrimidin-1(2H)-yl)piperidine-1-carboxylate (0.568 g, Yield Qnt) as a yellowish brown sticky liquid. LCMS purity 100% (Method A).

### Step-3: Synthesis of (R)-1-(piperidin-3-yl)tetrahydropyrimidin-2(1H)-one

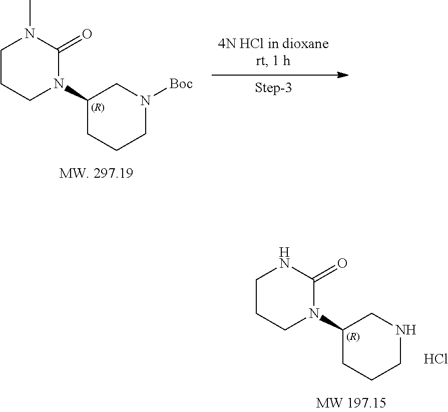

To a stirred solution of tert-butyl (R)-3-(3-methyl-2-oxotetrahydropyrimidin-1(2H)-yl)piperidine-1-carboxylate (0.568 g) in DCM (5 mL) was added 4N in dioxane (1.5 ml) at room temperature and allow to stir for 1 h at room temperature. The reaction mixture was directly concentrated under reduced pressure to afford (R)-1-(piperidin-3-yl)tetrahydropyrimidin-2(1H)-one (0.65 g, Yield Qnt.) as a brownish sticky solid. LCMS purity 98.79% (Method A).

### Step-4: Synthesis of 4-nitrophenyl (R)-3-(3-methyl-2-oxotetrahydropyrimidin-1(2H)-yl)piperidine-1-carboxylate

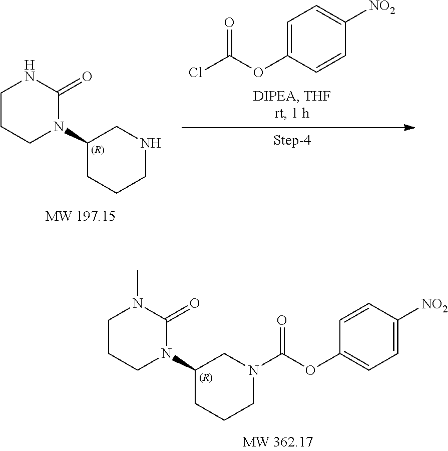

To a stirred solution of (R)-1-(piperidin-3-yl)tetrahydropyrimidin-2(1H)-one (0.65 g, 0.0033 mol) in THF (6.0 mL) was added DIPEA (1.73 ml, 0.0099 mol) and the reaction mixture was allowed to stir at room temperature for 20 min. 4-nitrophenyl chloroformate (0.66 g, 0.0033 mol) was then added to reaction mixture and stirred at room temperature for one hour. The reaction progress was monitored using TLC (mobile phase: neat ethyl acetate). The reaction mixture was diluted with water (50 mL) and extracted with ethyl acetate (50×2 mL). The combined organic layer was dried over sodium sulphate, filtered and concentrated under reduced pressure to afford 4-nitrophenyl(R)-3-(3-methyl-2-oxotetrahydropyrimidin-1(2H)-yl)piperidine-1-carboxylate (0.220 g, Yield 18.29%) as a pale yellowish brown sticky liquid. LCMS purity 98.79% (Method A).

### Step-5: Synthesis of (R)—N—((S)-3-(3,4-dihydroisoquinolin-2(1H)-yl)-2-hydroxypropyl)-3-(3-methyl-2-oxotetrahydropyrimidin-1(2H)-yl)piperidine-1-carboxamide

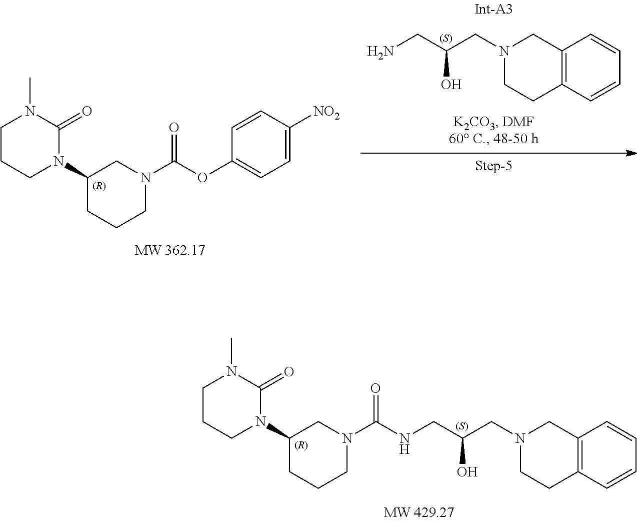

To a stirred solution of 4-nitrophenyl(R)-3-(3-methyl-2-oxotetrahydropyrimidin-1(2H)-yl)piperidine-1-carboxylate (0.22 g, 0.0006 mol) in DMF (1.5 mL) was added K₂CO₃ (0.33 g, 0.0024 mol) and Int-A3 (0.18 g, 0.0009 mol). The reaction mixture was heated at 65° C. for 48-50 h. The reaction mixture was diluted with water (60 mL) and extracted with ethyl acetate (60×2 mL). The combined organic layer was then washed with chilled brine (60 mL×3) and then dried over anhydrous sodium sulphate, filtered and concentrated under reduce pressure to afford (R)—N—((S)-3-(3,4-dihydroisoquinolin-2(1H)-yl)-2-hydroxypropyl)-3-(3-methyl-2-oxotetrahydropyrimidin-1(2H)-yl)piperidine-1-carboxamide (0.104 g, Yield 39.88%) as a white solid. LCMS purity 99.50% (Method B). 1HNMR: 400 MHZ, DMSO-d6: 7.094-7.011 (4H, m), 6.562-6.537 (1H, t), 4.839-4.828 (d, J=4.4 Hz, 1H), 4.016-3.976 (1H, m), 3.814-3.735 (3H, m), 3.638-3.546 (2H, m), 3.194-3.097 (5H, m), 3.082-2.968 (1H, m), 2.790-2.731 (5H, s), 2.718-2.676 (4H, m), 2.662-2.592 (2H, m), 1.833-1.804 (2H, m), 1.789-1.529 (3H, s), 1.340 (1H, m).

### Example 9

### Preparation of (R)—N—((S)-3-(3,4-dihydroisoquinolin-2(1H)-yl)-2-hydroxypropyl)-3-(3-methyl-2-oxopyrazin-1(2H)-yl)piperidine-1-carboxamide

The synthetic steps shown in the reaction scheme below were performed.

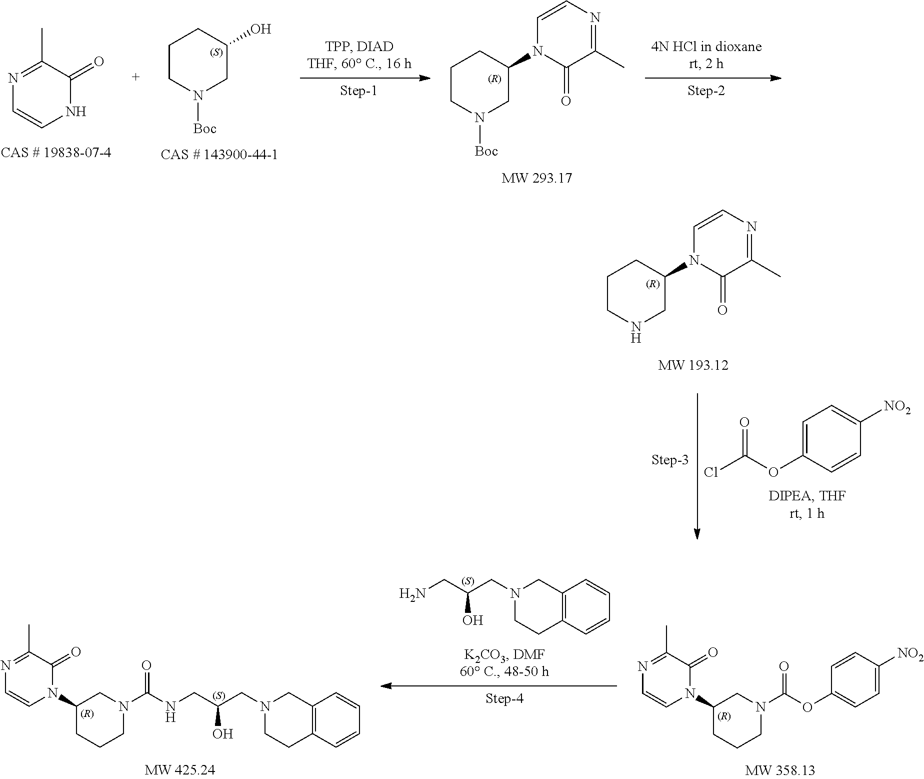

### Step-1: Synthesis of tert-butyl (R)-3-(3-methyl-2-oxopyrazin-1(2H)-yl)piperidine-1-carboxylate

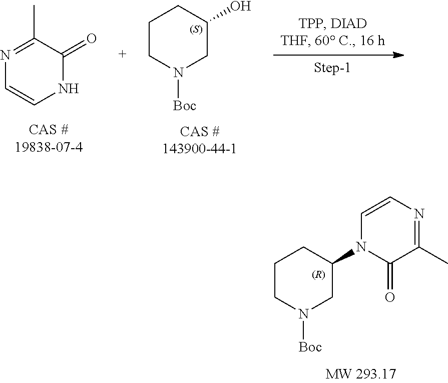

To a stirred solution of CAS No. 143900-44-1 (0.500 g, 0.0025 mol) and CAS No. 19838-07-4 (0.275 g, 0.0025 mol) in dry THF (5 mL) TPP (1.31 g, 0.005 mol) was added. The reaction mixture was allowed to stir at 70° C. for 30 min, after that DIAD (1.50 g, 0.0075 mol) was added dropwise over period of 10 min. The reaction mixture was allowed to stir at 70° C. for 16 h. Reaction progress was monitored using TLC (mobile phase: 20% ethyl acetate:hexane). The reaction mixture was diluted with water (100 mL) and extracted with ethyl acetate (100 mL×2). The combined organic layer was then dried over anhydrous sodium sulphate, filtered and concentrated under reduced pressure. The crude material was then purified by silica gel (60-120 mesh) column chromatography using 20% ethyl acetate in hexane to afford tert-butyl (R)-3-(3-methyl-2-oxopyrazin-1(2H)-yl)piperidine-1-carboxylate (0.300 g, Yield 22.52%) as a colourless liquid. LCMS purity 96.00% (Method A).

### Step-2: Synthesis of (R)-3-methyl-1-(piperidin-3-yl)pyrazin-2(1H)-one

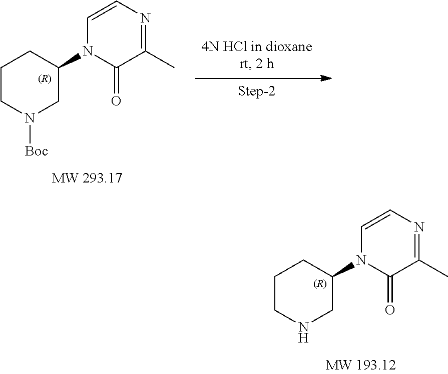

To a stirred solution of tert-butyl (R)-3-(3-methyl-2-oxopyrazin-1(2H)-yl)piperidine-1-carboxylate (0.300 g, 0.0010 mol) in DCM (5 mL) was added 4N HCl in dioxane (2 mL) at room temperature. The reaction mixture was allowed to stir at room temperature for 2 h. Reaction mixture was directly concentrated under reduced pressure to afford (R)-3-methyl-1-(piperidin-3-yl)pyrazin-2(1H)-one (0.250 g, Yield Qnt.) as a white sticky solid. Crude material was directly used for next step without further purification. LCMS purity 89.56% (Method A).

### Step-3: Synthesis of 4-nitrophenyl (R)-3-(3-methyl-2-oxopyrazin-1(2H)-yl)piperidine-1-carboxylate

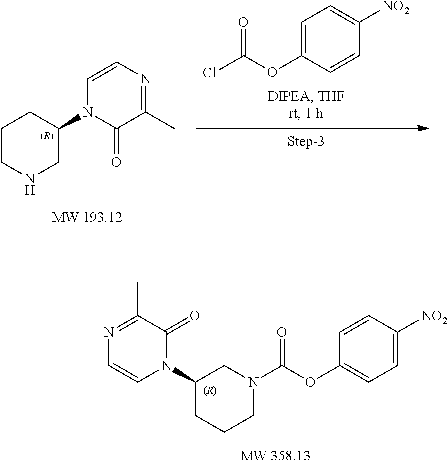

To a stirred solution of (R)-3-methyl-1-(piperidin-3-yl)pyrazin-2(1H)-one (0.250 g, 0.0013 mol) in THF (5 mL) was added DIPEA (0.670 g, 0.0052 mol) at room temperature and stirred for 15 min followed by addition of 4-nitrophenyl chloroformate (0.316 g, 0.0015 mol). The reaction mixture was allowed to stir at room temperature for 1 h. Reaction mixture was diluted with water (50 mL) and extracted with ethyl acetate (50 mL×2). The combined organic layer was dried over anhydrous sodium sulphate, filtered and concentrated under reduced pressure. The obtained crude was purified by silica gel (60-120 mesh) column chromatography using 50% ethyl acetate in hexane to afford 4-nitrophenyl (R)-3-(3-methyl-2-oxopyrazin-1(2H)-yl)piperidine-1-carboxylate (0.200 g, Yield 43.14%) as a yellow sticky solid. LCMS purity 92.71% (Method A).

### Step-4: Synthesis of (R)—N—((S)-3-(3,4-dihydroisoquinolin-2(1H)-yl)-2-hydroxypropyl)-3-(3-methyl-2-oxopyrazin-1(2H)-yl)piperidine-1-carboxamide

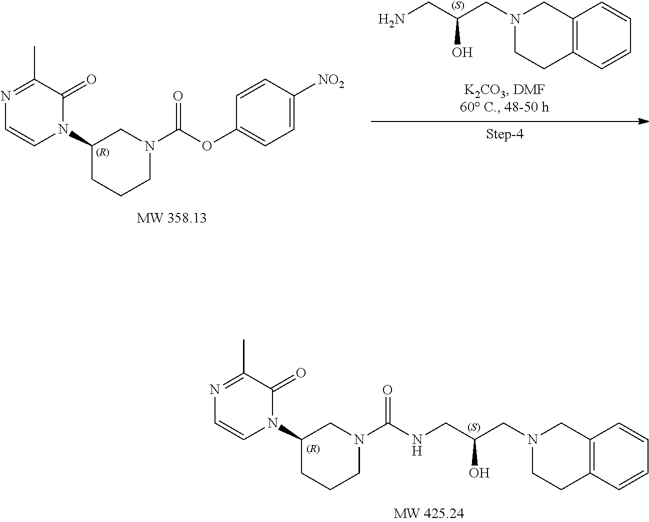

To a stirred solution of 4-nitrophenyl (R)-3-(3-methyl-2-oxopyrazin-1(2H)-yl)piperidine-1-carboxylate (0.200 g, 0.0005 mol) in DMF (3.0 mL) was added Int-A3 (0.138 g, 0.0006 mol) and potassium carbonate (0.278 g, 0.002 mol) at room temperature. Reaction mixture was heated at 65° C. for 48-50 h. The reaction progress was monitored using TLC (mobile phase: 10% MeOH in DCM). After consumption of 4-nitrophenyl (R)-3-(3-methyl-2-oxopyrazin-1(2H)-yl)piperidine-1-carboxylate, the reaction mixture was diluted with ice cold water (100 mL) and extracted with ethyl acetate (100 mL×2). Combined organic layer was then washed with chilled brine (100 mL×3). Organic layer was dried over anhydrous sodium sulphate, filtered and concentrated under reduced pressure. The obtained crude was purified by reverse phase flash chromatography using 0.1% ammonia solution in water:acetonitrile to afford (R)—N—((S)-3-(3,4-dihydroisoquinolin-2(1H)-yl)-2-hydroxypropyl)-3-(3-methyl-2-oxopyrazin-1(2H)-yl)piperidine-1-carboxamide (0.08 g, Yield 33.69%). LCMS purity 95.14% (Method B). 1H NMR: 400 MHZ, DMSO-d6: 8.02-7.99 (2H, m), 7.08-7.06 (3H, m), 7.00-6.99 (m, 1H), 6.54 (m, 1H), 4.96 (m, 1H), 4.80-4.79 (d, J=4 Hz, 1H), 3.71-3.70 (m, 1H), 3.61-3.52 (m, 3H), 3.45-3.33 (m, 1H), 3.29-3.22 (m, 2H), 3.11-2.98 (m, 2H), 2.77-2.76 (m, 2H), 2.73-2.62 (m, 3H), 2.44-2.38 (m, 2H), 2.32 (s, 3H), 1.95-1.92 (m, 1H), 1.73-1.69 (m, 2H), 1.39 (br s, 1H).

### Example 10

### Preparation of (R)-3-(5-chloro-6-oxopyridazin-1(6H)-yl)—N—((S)-3-(3,4-dihydroisoquinolin-2(1H)-yl)-2-hydroxypropyl)piperidine-1-carboxamide

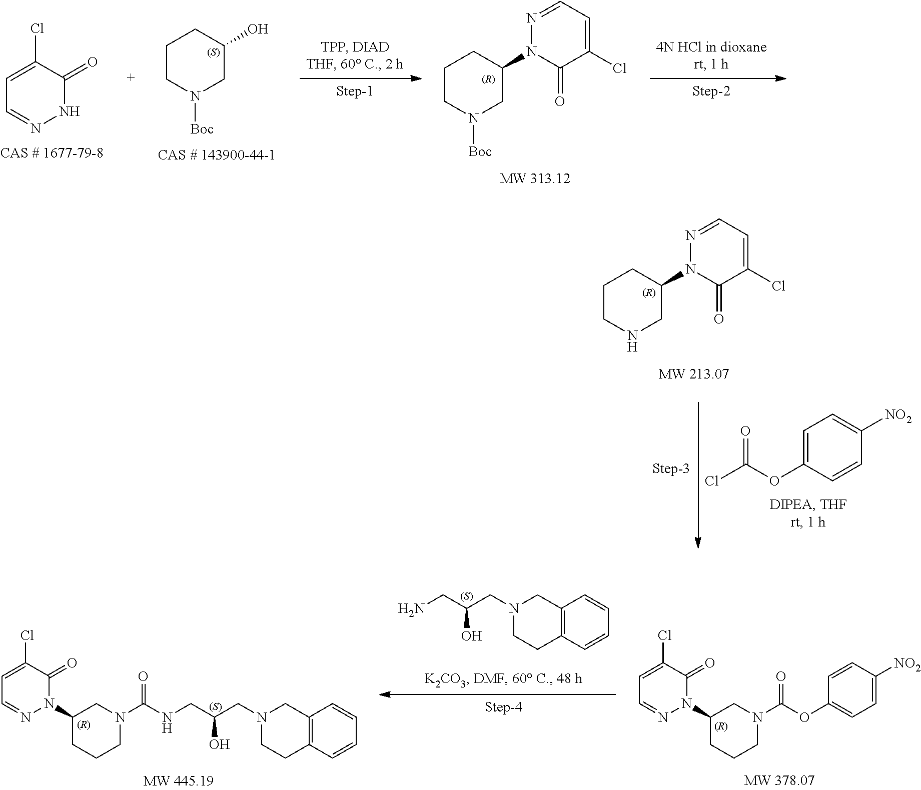

### Step-1: Synthesis of tert-butyl (R)-3-(5-chloro-6-oxopyridazin-1(6H)-yl)piperidine-1-carboxylate

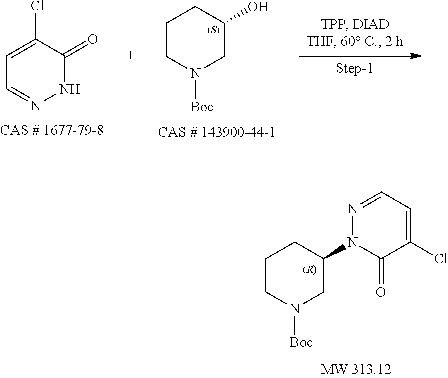

To a stirred solution of CAS No. 1677-79-8 (0.162 g, 1.24 mmol) and CAS No. 143900-44-41 (0.250 g, 1.24 mmol) in dry THF (2.5 mL), TPP (0.488 g, 1.86 mmol) was added. The reaction mixture was allowed to stir at 60° C. for 30 min, after that DIAD (0.377 g, 1.86 mmol) was added dropwise over period of 10 min. The reaction mixture was allowed to stir at 60° C. for 2 h. The reaction mixture was cooled to room temperature and diluted with water (30 mL). The suspension was extracted with ethylacetate (30 mL×2). The combined organic layer was dried over anhydrous sodium sulphate, filtered and concentrated under reduced pressure. The obtained crude was purified by silica gel (60-120 mesh) column chromatography using 12% ethylacetate in hexane to afford tert-butyl (R)-3-(5-chloro-6-oxopyridazin-1(6H)-yl)piperidine-1-carboxylate (0.25, Yield: 41.60%) as colorless sticky liquid. LCMS purity 94.68% (Method A).

### Step-2: (R)-4-chloro-2-(piperidin-3-yl)pyridazin-3(2H)-one

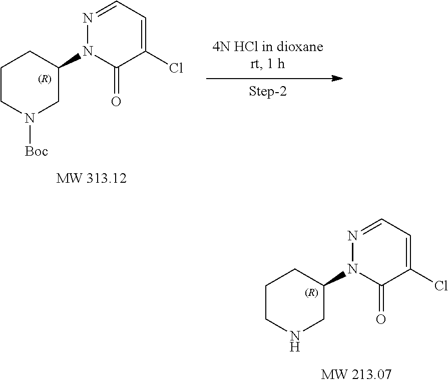

To a stirred solution of tert-butyl (R)-3-(5-chloro-6-oxopyridazin-1(6H)-yl)piperidine-1-carboxylate (0.25 g, 0.79 mmol) in DCM (2.5 mL) was added 4N HCl in dioxane (3 mL) at room temperature. The reaction mixture was allowed to stir at room temperature for 30 min. The reaction mixture was concentrated under reduced pressure to afford ((R)-4-chloro-2-(piperidin-3-yl)pyridazin-3(2H)-one (0.49 g, Yield: Qnt.) as a white sticky solid. The crude material was directly used for the next step without further purification. LCMS purity 50.42%.

### Step-3: Synthesis of 4-nitrophenyl (R)-3-(5-chloro-6-oxopyridazin-1(6H)-yl)piperidine-1-carboxylate

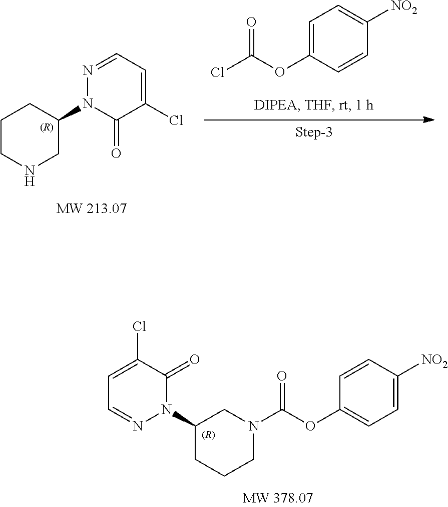

To a stirred solution of (R)-4-chloro-2-(piperidin-3-yl)pyridazin-3(2H)-one (0.49 g, 2.33 mmol) in THF (4 mL) was added DIPEA (1.8 g, 9.32 mol) at room temperature and stirred for 15 min followed by addition of 4-nitrophenyl chloroformate (0.554 g, 2.33 mmol). The reaction mixture was allowed to stir at room temperature for 1 h. The reaction mixture was diluted with water (50 mL) and extracted with ethyl acetate (50 mL×2). The combined organic layer was dried over anhydrous sodium sulphate, filtered and concentrated under reduced pressure. The obtained crude was purified by silica gel (60-120 mesh) column chromatography using 22% ethyl acetate to produce 4-nitrophenyl (R)-3-(5-chloro-6-oxopyridazin-1(6H)-yl) piperidine-1-carboxylate (0.2 g, Yield: 45.13%) as white sticky solid. LCMS purity 100% (Method A).

### Step-4: Synthesis of (R)-3-(5-chloro-6-oxopyridazin-1(6H)-yl)—N—((S)-3-(3,4-dihydroisoquinolin-2(1H)-yl)-2-hydroxypropyl)piperidine-1-carboxamide

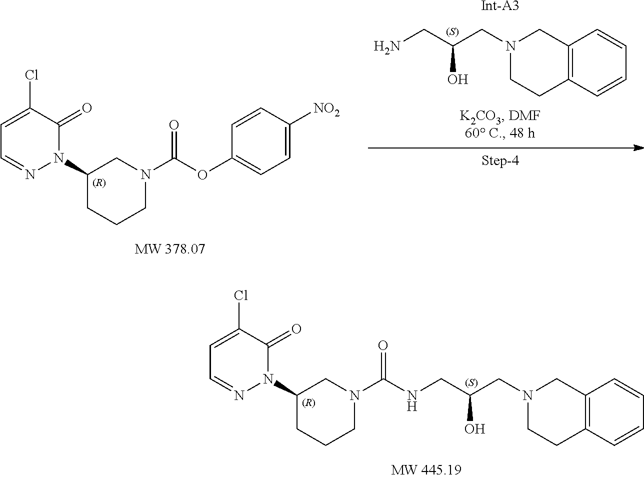

To a stirred solution of 4-nitrophenyl (R)-3-(5-chloro-6-oxopyridazin-1(6H)-yl)piperidine-1-carboxylate (0.1 g, 0.26 mmol) in DMF (2.0 mL) was added Int-A3 (0.065 g, 0.39 mmol) and potassium carbonate (0.146 g, 1.05 mmol) at room temperature. Reaction mixture was heated at 65° C. for 48 h. After completion, the reaction mixture was cooled to room temperature and diluted with ice cold water (50 mL). The resulting suspension was extracted with ethyl acetate (50 mL×2). The combined organic layer was washed with chilled brine (100 mL×3). Organic layer was dried over anhydrous sodium sulphate, filtered and concentrated under reduced pressure. The obtained crude was purified by reverse phase flash chromatography using 0.1% ammonia solution in water:acetonitrile to afford (R)-3-(5-chloro-6-oxopyridazin-1(6H)-yl)—N—((S)-3-(3,4-dihydroisoquinolin-2(1H)-yl)-2-hydroxypropyl)piperidine-1-carboxamide (0.027 g, Yield: 22.93%). 1H NMR: 400 MHZ, DMSO-d6: 7.949-7.938 (d, J=4 Hz, 1H), 7.793-7.782 (d, J=4 Hz, 1H), 7.085-7.015 (m, 4H), 6.683 (s, 1H), 4.816 (s, 1H), 4.725-4.670 (t, 1H), 4.069-4.040 (d, J=11 Hz, 1H), 3.899-3.865 (d, J=13, 1H), 3.768 (Br, 1H), 3.639-3.548 (m, 2H), 3.217-3.157 (m, 1H), 3.025-2.963 (m, 3H), 2.919-2.858 (t, 1H), 2.778-2.747 (m, 2H), 2.728-2.6081 (m, 4H), 2.559-2.541 (m, 3H), 2.467-2.325 (m, 1H), 1.896 (Br, 1H), 1.771-1.663 (m, 1H), 1.469-1.437 (m, 1H).

### Example 11

### Preparation of (R)—N—((S)-3-(3,4-dihydroisoquinolin-2(1H)-yl-1,1,4,4-d4)-2-hydroxypropyl)-3-(5-methyl-6-oxopyridazin-1(6H)-yl)piperidine-1-carboxamide

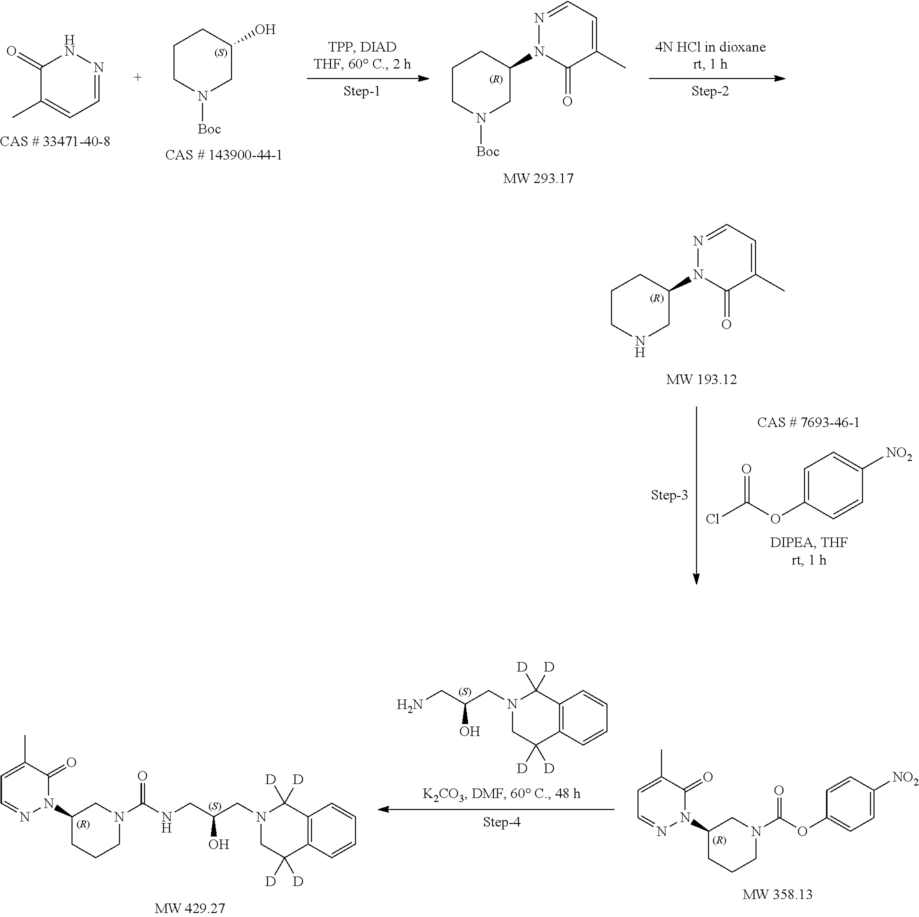

### Step-1: Synthesis of tert-butyl (R)-3-(5-methyl-6-oxopyridazin-1(6H)-yl)piperidine-1-carboxylate

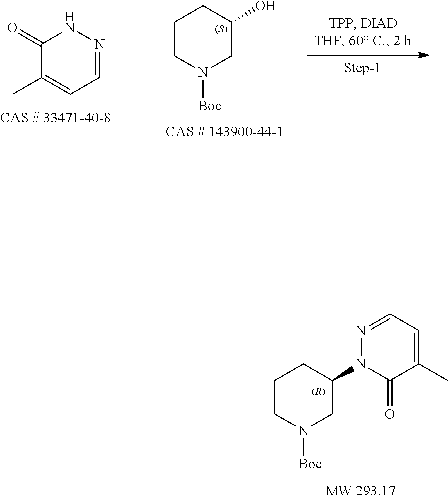

To a stirred solution of CAS No. 33471-40-8 (0.27 g, 2.48 mmol) and CAS No. 143900-44-41 (0.5 g, 2.48 mmol) in dry THF (5.0 mL) TPP (0.97 g, 3.73 mmol) was added. The reaction mixture was allowed to stir at 60° C. for 30 min, after that DIAD (0.75 g, 3.73 mmol) was added dropwise over period of 10 min. The reaction mixture was allowed to stir at 60° C. for 2 h. After completion, the reaction mixture was cooled to room temperature and diluted with water (50 mL). The resulting suspension was extracted with ethyl acetate (50 mL×2). The combined organic layer was dried over anhydrous sodium sulphate, filtered and concentrated under reduce pressure. The obtained crude was purified by silica gel (60-120 mesh) column chromatography using 20% ethylacetate in hexane to afford tert-butyl (R)-3-(5-methyl-6-oxopyridazin-1(6H)-yl)piperidine-1-carboxylate (0.35 g, Yield: 26.27%) as yellow sticky liquid. LCMS purity: 100% (Method A).

### Step-2: Synthesis of (R)-4-methyl-2-(piperidin-3-yl)pyridazin-3(2H)-one

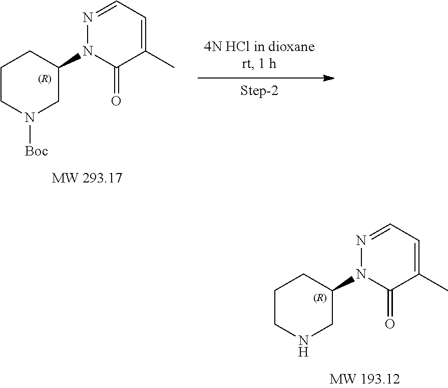

To a stirred solution of tert-butyl (R)-3-(5-methyl-6-oxopyridazin-1(6H)-yl)piperidine-1-carboxylate (0.35 g, 1.10 mmol) in DCM (2.0 mL) was added 4N HCl in dioxane (2.0 mL) at room temperature. The reaction mixture was allowed to stir at room temperature for 1 h. Reaction mixture was concentrated under reduce pressure to afford (R)-4-methyl-2-(piperidin-3-yl)pyridazin-3(2H)-one (0.3 g, Yield: Qnt.) as a white sticky solid. Crude material was directly used for next step without further purification.

### Step-3: Synthesis of 4-nitrophenyl (R)-3-(5-methyl-6-oxopyridazin-1(6H)-yl)piperidine-1-carboxylate

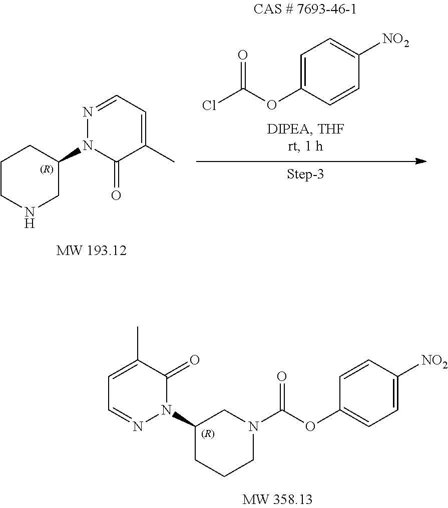

To a stirred solution of (R)-4-methyl-2-(piperidin-3-yl)pyridazin-3(2H)-one (0.3 g, 1.55 mmol) in THF (3 mL) was added DIPEA (0.80 g, 6.20 mmol) at room temperature and stirred for 15 min followed by addition of 4-nitrophenyl chloroformate (0.31 g, 1.55 mmol). The reaction mixture was allowed to stir at room temperature for 1 h. Reaction mixture was diluted with water (30 mL) and extracted with ethyl acetate (50 mL×2). The combined organic layer was dried over anhydrous sodium sulphate, filtered and concentrated under reduced pressure. The obtained crude was purified by silica gel (60-120 mesh) column chromatography using 60% Ethyl acetate in Hexane to afford 4-nitrophenyl (R)-3-(5-methyl-6-oxopyridazin-1(6H)-yl)piperidine-1-carboxylate (0.28 g, Yield: 43.14%) as brown sticky solid. LCMS purity: 100% (Method A).

### Step-4: Synthesis of (R)-4-methyl-2-(piperidin-3-yl)pyridazin-3(2H)-one

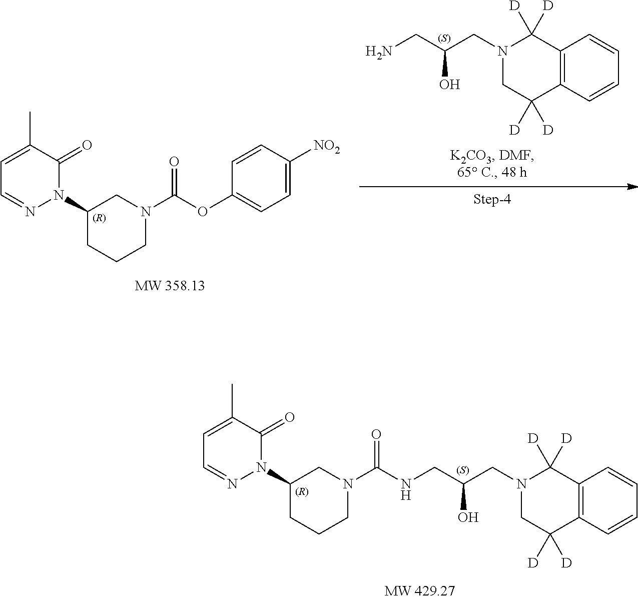

To a stirred solution of 4-nitrophenyl (R)-3-(5-methyl-6-oxopyridazin-1(6H)-yl)piperidine-1-carboxylate (0.1 g, 0.27 mmol) in DMF (2.0 mL) was added Int-A3 (0.07 g, 0.33 mmol) and potassium carbonate (0.154 g, 1.11 mmol) at room temperature. Reaction mixture was heated at 65° C. for 48 h. After completion, the reaction mixture was cooled to room temperature and diluted with ice cold water (50 mL). The resulting suspension was extracted with ethyl acetate (50 mL×2). Combined organic layer was washed with chilled brine (100 mL×3). Organic layer was dried over anhydrous sodium sulphate, filtered and concentrated under reduced pressure. The obtained crude was purified by reverse phase flash chromatography using 0.1% ammonia solution in water:acetonitrile to afford (R)—N—((S)-3-(3,4-dihydroisoquinolin-2(1H)-yl-1,1,4,4-d4)-2-hydroxypropyl)-3-(5-methyl-6-oxopyridazin-1(6H)-yl)piperidine-1-carboxamide (0.02 g, Yield: 16.68%). 1H NMR: 400 MHZ, DMSO-d6: 7.852-7.842 (d, J=4 Hz, 1H), 7.298-7.290 (d, J=4 Hz, 1H), 7.110-7.070 (m, 2H), 7.056-7.017 (m, 1H), 6.687-6.664 (t, 1H), 4.817-4.807 (d, J=4 Hz, 1H), 4.750-4.696 (m, 1H), 4.039-4.010 (d, J=11 Hz, 1H), 3.924-3.891 (d, J=13 Hz, 1H), 3.789-3.776 (m, 1H), 3.227-3.168 (m, 1H), 3.040-2.993 (m, 1H), 2.912-2.853 (t, 1H), 2.744-2.574 (m, 3H), 2.477-2.337 (m, 2H), 2.096 (s, 3H), 1.852-1.783 (m, 2H), 1.696-1.662 (m, 1H), 1.477-1.445 (m, 1H).

### Example 12

### Preparation of (R)—N—((S)-3-(3,4-dihydroisoquinolin-2(1H)-vl-1,1,4,4-d4)-2-hydroxypropyl)-3-(3-methyl-2-oxopyrazin-1(2H)-yl)piperidine-1-carboxamide

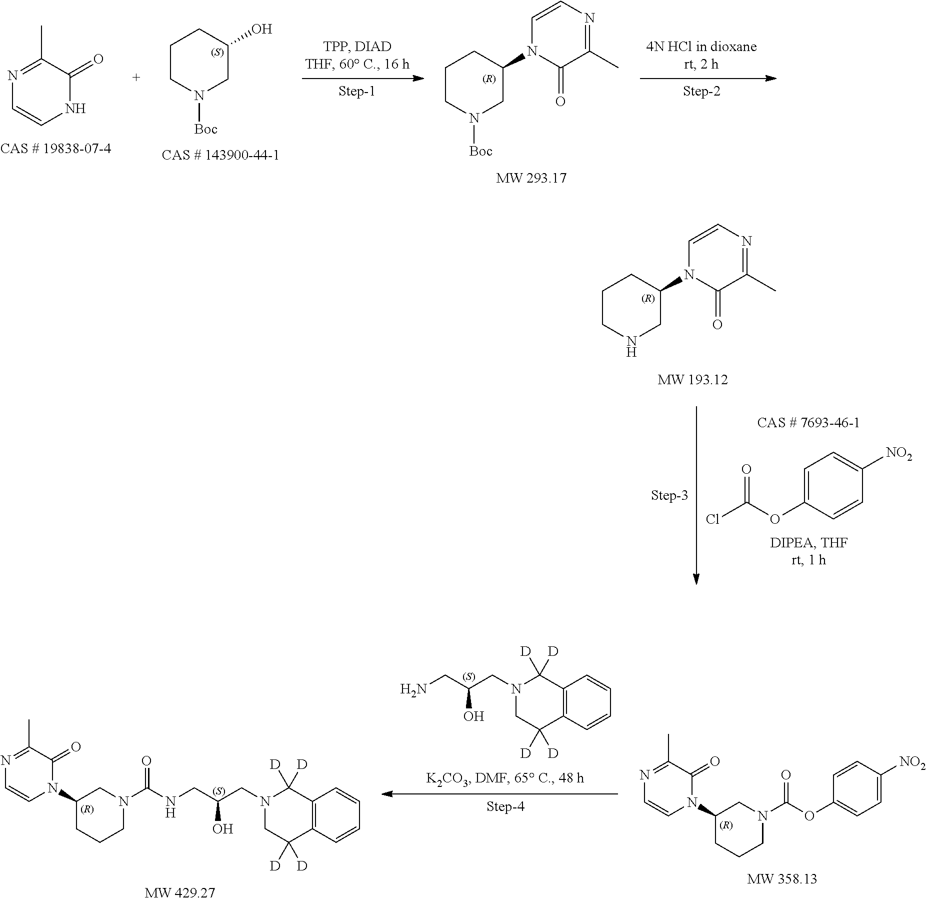

### Step-1: Synthesis of tert-butyl (R)-3-(3-methyl-2-oxopyrazin-1(2H)-yl)piperidine-1-carboxylate

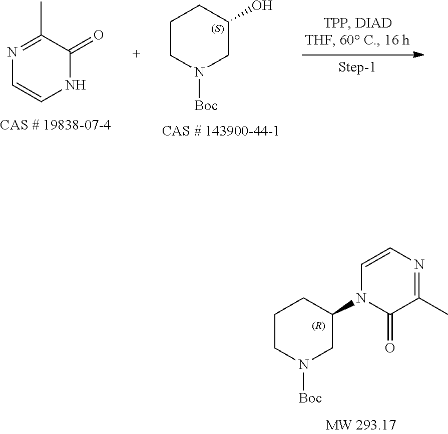

In the first step, tert-butyl (R)-3-(3-methyl-2-oxopyrazin-1(2H)-yl)piperidine-1-carboxylate was prepared as described in Step-1 of Example 9.

### Step-2: Synthesis of (R)-3-methyl-1-(piperidin-3-yl)pyrazin-2(1H)-one

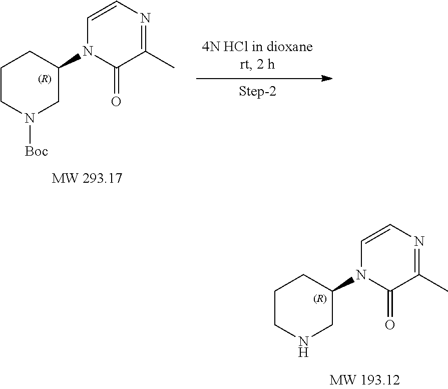

In the second step, (R)-3-methyl-1-(piperidin-3-yl)pyrazin-2(1H)-onetert-butyl (R)-3-(3-methyl-2-oxopyrazin-1(2H)-yl)piperidine-1-carboxylate was prepared as described in Step-2 of Example 9.

### Step-3: Synthesis of 4-nitrophenyl (R)-3-(3-methyl-2-oxopyrazin-1(2H)-yl)piperidine-1-carboxylate

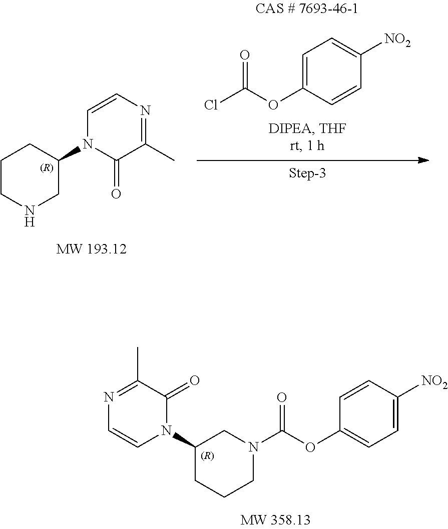

In the third step, 4-nitrophenyl (R)-3-(3-methyl-2-oxopyrazin-1(2H)-yl)piperidine-1-carboxylate was prepared as described in Step-3 of Example 9.

### Step-4: Synthesis of (R)—N—((S)-3-(3,4-dihydroisoquinolin-2(1H)-yl-1,1,4,4-d4)-2-hydroxypropyl)-3-(3-methyl-2-oxopyrazin-1(2H)-yl)piperidine-1-carboxamide

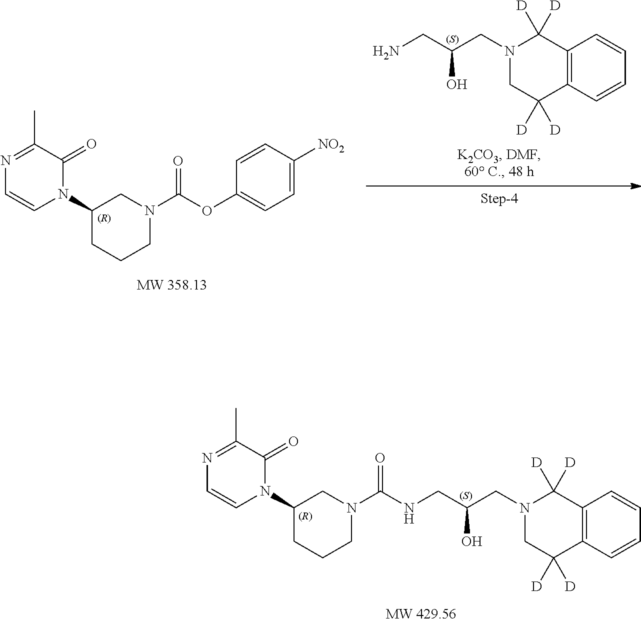

To a stirred solution of 4-nitrophenyl (R)-3-(3-methyl-2-oxopyrazin-1(2H)-yl)piperidine-1-carboxylate (0.1 g, 0.27 mmol) in DMF (5 mL) was added Int-A3 (0.070 g, 0.33 mmol) and potassium carbonate (0.15 g, 1.32 mmol) at room temperature. Reaction mixture was heated at 65° C. for 48 h. After completion, the reaction mixture was cooled to room temperature and diluted with ice cold water (60 mL). The resulting suspension was extracted with ethyl acetate (60 mL×2) The combined organic layer was washed with chilled brine (60 mL×3). The organic layer was dried over anhydrous sodium sulphate, filtered and concentrated under reduced pressure. The obtained crude was purified by reverse phase flash chromatography using 0.1% ammonia solution in water:acetonitrile to afford (R)—N—((S)-3-(3,4-dihydroisoquinolin-2(1H)-yl)-2-hydroxypropyl)-3-(3-methyl-2-oxopyrazin-1(2H)-yl)piperidine-2,2,5,5-d4-1-carboxamide (0.04 g, Yield: 33.37%) yellow sticky solid. 1H NMR: 400 MHZ, DMSO-d6: 8.045-8.014 (m, 2H), 7.111-7.008 (m, 4H), 6.542 (br s, 1H), 4.987 (br s, 1H), 4.799-4.789 (d, J=4 Hz, 1H), 3.731-3.721 (m, 1H), 3.601-3.545 (m, 2H), 3.482-3.434 (m, 1H), 3.138-2.970 (m, 2H), 2.806-2.641 (m, 3H), 2.453-2.349 (m, 3H), 2.292 (s, 3H), 1.963-1.941 (m, 2H), 1.753-1.724 (m, 2H), 1.416 (br m, 1H).

### Example 13

### Preparation of (R)—N—((S)-3-(3,4-dihydroisoquinolin-2(1H)-yl)-2-hydroxypropyl)-3-(2-oxoazepan-1-yl)piperidine-1-carboxamide

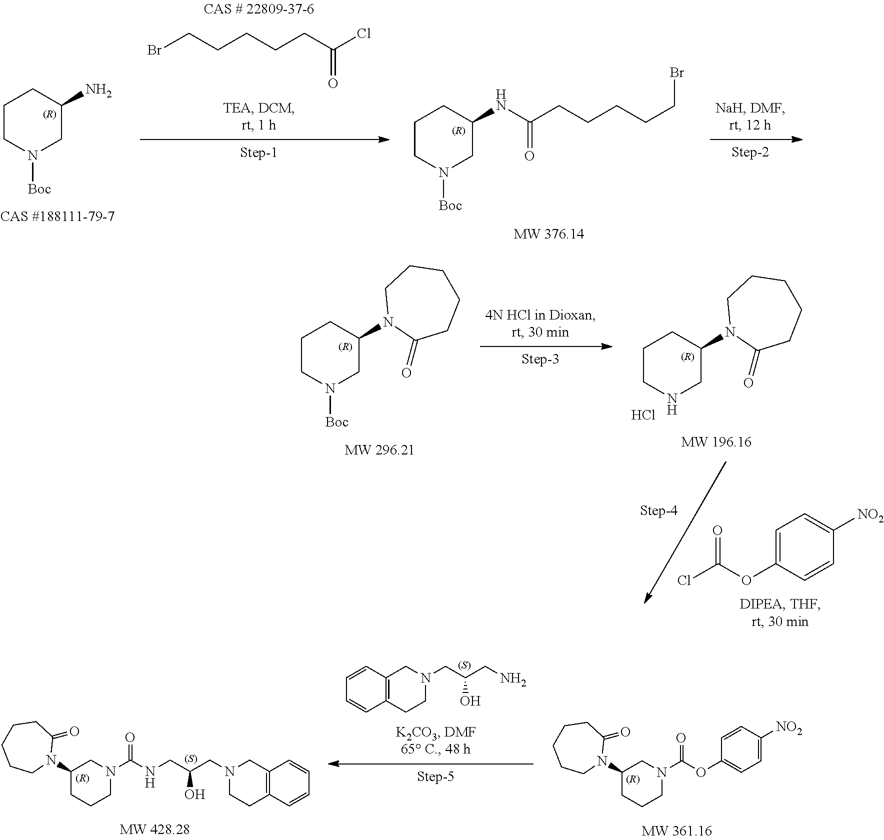

### Step-1: Synthesis of tert-butyl (R)-3-(6-bromohexanamido)piperidine-1-carboxylate

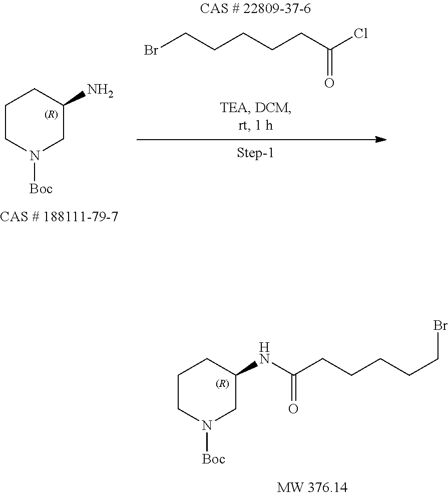

In an oven dried single neck round bottom flask was charged a solution of CAS No. 188111-79-7 (0.5 g, 2.50 mmol) in DCM (5 mL). The reaction mixture was stirred at 0° C. and TEA (0.3 g, 2.70 mmol) was added and further reaction mixture was allowed to stir for 10 min. CAS #22809-37-6 (0.58 g, 2.70 mmol) was added drop wise and resulting reaction mixture was allowed to stir at RT for 1 h. After completion, the reaction mixture was diluted with water (50 mL) and extracted with DCM (60 mL×2). The combined organic layer was dried over anhydrous sodium sulphate, filtered and concentrated under reduce pressure to afford tert-butyl (R)-3-(6-bromohexanamido)piperidine-1-carboxylate (1.0 g, Yield:Qut.) as brown sticky liquid. LCMS purity: 100% (Method A).

### Step-2: Synthesis of tert-butyl (R)-3-(2-oxoazepan-1-yl)piperidine-1-carboxylate

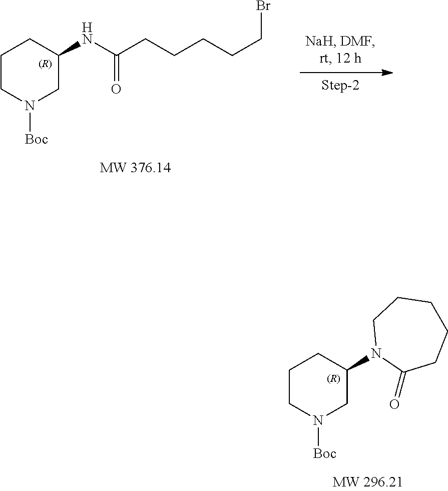

To a stirred solution of tert-butyl (R)-3-(6-bromohexanamido)piperidine-1-carboxylate (1.0 g, 2.65 mmol) in dry DMF (7.0 mL) at 0° C. NaH (0.127 g, 5.31 mmol) was added portion wise. The reaction mixture was allowed to stir at room temperature for 12 h. The reaction mixture was diluted with ice cold water (60 mL) and extracted with ethyl acetate (60 mL×2). The combined organic layer was dried over anhydrous sodium sulphate, filtered and concentrated under reduced pressure. The obtained crude was purified by silica gel (60-120 mesh) column chromatography using 22% ethyl acetate in hexane to afford tert-butyl (R)-3-(2-oxoazepan-1-yl)piperidine-1-carboxylate (0.68 g, Yield: 86.56%) as brown sticky liquid. LCMS purity: 76.44% (Method A).

### Step-3: Synthesis of (R)-1-(piperidin-3-yl)azepan-2-one

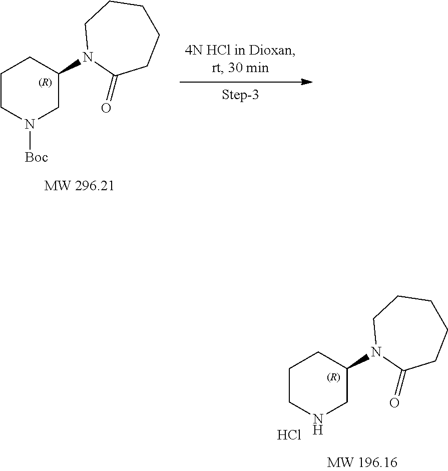

To a stirred solution of tert-butyl (R)-3-(2-oxoazepan-1-yl)piperidine-1-carboxylate (0.68 g, 2.20 mmol) in DCM (4.0 mL) was added 4N HCl in dioxane (4.0 mL) at room temperature. The reaction mixture was allowed to stir at room temperature for 30 min. The reaction mixture was concentrated under reduced pressure to afford (R)-1-(piperidin-3-yl)azepan-2-one (0.6 g, Yield: Qnt.) as a white sticky solid. Crude material was directly used for next step without further purification. LCMS purity: 93.26% (Method A).

### Step-4: Synthesis of 4-nitrophenyl (R)-3-(2-oxoazepan-1-yl)piperidine-1-carboxylate

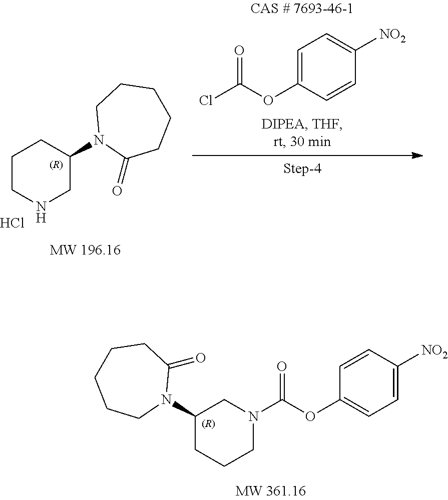

To a stirred solution of (R)-1-(piperidin-3-yl)azepan-2-one (0.6 g, 3.06 mmol) in THF (6.0 mL) was added DIPEA (1.57 g, 12.24 mmol) at room temperature and stirred for 15 min followed by addition of 4-Nitro phenyl chloroformate (0.61 g, 3.06 mmol). The reaction mixture was allowed to stir at room temperature for 30 min. Reaction mixture was diluted with water (50 mL) and extracted with ethyl acetate (50 mL×2). The combined organic layer was dried over anhydrous sodium sulphate, filtered and concentrated under reduced pressure. The obtained crude was purified by silica gel (60-120 mesh) column chromatography using 40% ethyl acetate in hexane to afford 4-nitrophenyl (R)-3-(2-oxoazepan-1-yl)piperidine-1-carboxylate (0.6 g, Yield: 54.32%) as brown sticky solid. LCMS purity: 54.53% (Method A).

### Step-5: Synthesis of (R)—N—((S)-3-(3,4-dihydroisoquinolin-2(1H)-yl)-2-hydroxypropyl)-3-(2-oxoazepan-1-yl)piperidine-1-carboxamide

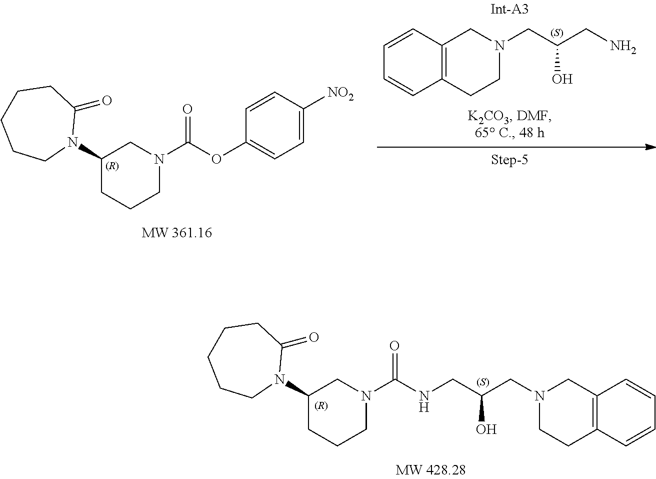

To a stirred solution of 4-nitrophenyl (R)-3-(2-oxoazepan-1-yl)piperidine-1-carboxylate (0.25 g, 0.69 mmol) in DMF (3.5 mL) was added Int-A3 (0.171 g, 0.83 mmol) and potassium carbonate (0.382 g, 2.77 mmol) at room temperature. Reaction mixture was then heated at 65° C. for 48 h. After completion, the reaction diluted with ice cold water (50 mL). The resulting suspension was extracted with ethyl acetate (50 mL×2). The combined organic layer was washed with chilled brine (50 mL×3). Organic layer was then dried over anhydrous sodium sulphate, filtered and concentrated under reduced pressure. The obtained crude was purified by reverse phase flash chromatography using 0.1% ammonia solution in water: Acetonitrile and prep HPLC to afford (R)—N—((S)-3-(3,4-dihydroisoquinolin-2(1H)-yl)-2-hydroxypropyl)-3-(2-oxoazepan-1-yl)piperidine-1-carboxamide (0.025 g, Yield: 8.46%). 1H NMR: 400 MHZ, DMSO-d6: 7.116-7.022 (m, 4H), 6.603-6.577 (m, 1H), 4.831-4.820 (d, J=4 Hz, 1H), 4.193 (br, 1H), 3.814-3.758 (m, 3H), 3.611-3.594 (m, 2H), 3.283-3.201 (m, 2H), 3.201-3.161 (m, 1H), 3.019-2.986 (m, 1H), 2.802-2.766 (m, 2H), 2.738-2.594 (m, 4H), 2.504-2.379 (m, 4H), 1.643-1.498 (m, 9H).

### Example 14

### Preparation of (R)—N—((S)-3-(3,4-dihydroisoquinolin-2(1H)-yl)-2-hydroxypropyl)-3-(3,5-dimethyl-2-oxopyrazin-1(2H)-yl)piperidine-1-carboxamide

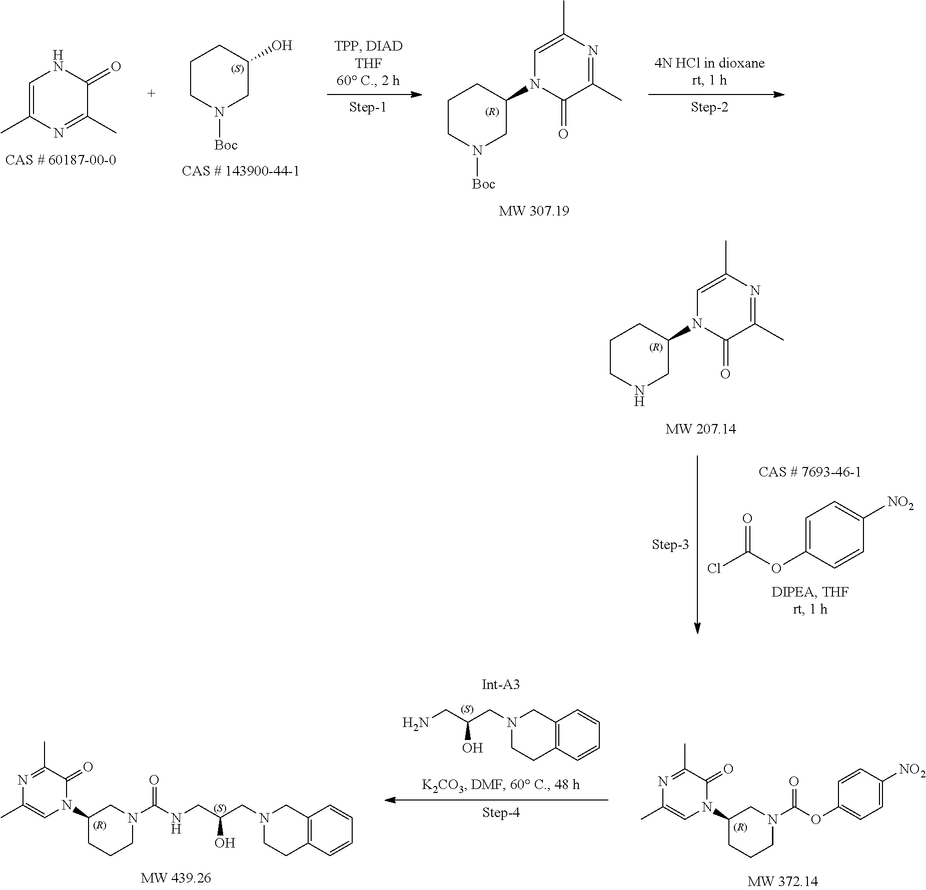

### Step-1: Synthesis of tert-butyl (R)-3-(3,5-dimethyl-2-oxopyrazin-1(2H)-yl)piperidine-1-carboxylate

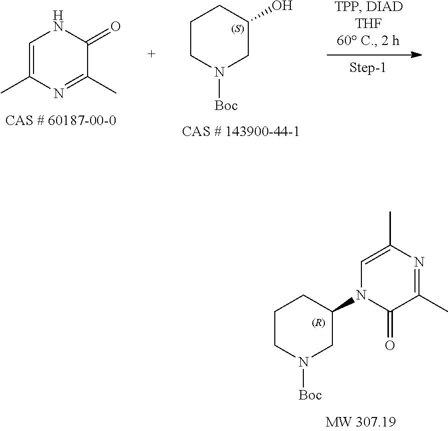

To a stirred solution of CAS No. 60187-00-0 (0.5 g, 2.48 mmol) and CAS No. 143900-44-1 (0.3 g, 2.48 mmol) in THF (4 mL), TPP (0.97 g, 3.73 mmol) was added. The reaction mixture was allowed to stir at 60° C. for 30 min, after that DIAD (0.75 g, 3.73 mmol) was added dropwise over period of 10 min. The reaction mixture was allowed to stir at 60° C. for 2 h. After completion, the reaction mixture was cooled to room temperature and diluted with water (100 mL). The resulting suspension was extracted with ethyl acetate (100 ml×2). The combined organic layer was then dried over anhydrous sodium sulphate, filtered and concentrated under reduce pressure. The crude material was then purified by silica gel (60-120 mesh) column chromatography using 7% ethylacetate in hexane to afford tert-butyl (R)-3-(3,5-dimethyl-2-oxopyrazin-1(2H)-yl)piperidine-1-carboxylate (0.26 g, Yield: 21%) as sticky solid. LCMS purity: 96.55% (Method A).

### Step-2: Synthesis of (R)-3,5-dimethyl-1-(piperidin-3-yl)pyrazin-2(1H)-one

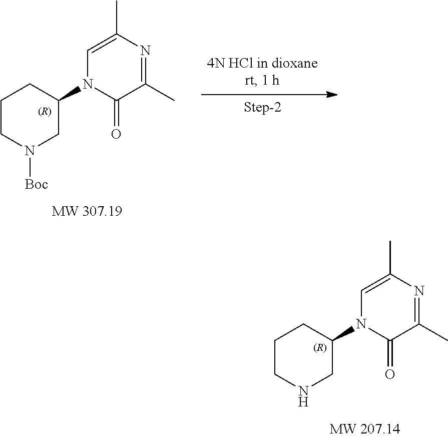

To a stirred solution of tert-butyl (R)-3-(3,5-dimethyl-2-oxopyrazin-1(2H)-yl)piperidine-1-carboxylate (0.26 g, 0.84 mmol) in DCM (5 mL) was added 4N HCl in dioxane (3 mL) at room temperature. The reaction mixture was allowed to stir at room temperature for 1 h. The reaction mixture was concentrated under reduce pressure to afford (R)-3,5-dimethyl-1-(piperidin-3-yl)pyrazin-2(1H)-one (0.25 g, Yield: Qnt.) as a white sticky solid. Crude material was directly used for next step without further purification. LCMS purity: 100% (Method A).

### Step-3: Synthesis of 4-nitrophenyl (R)-3-(5-methyl-2-oxopyrazin-1(2H)-yl)piperidine-1-carboxylate

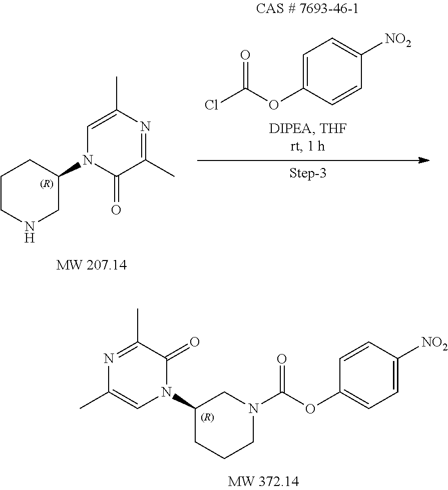

To a stirred solution of (R)-3,5-dimethyl-1-(piperidin-3-yl)pyrazin-2(1H)-one (0.23 g, 1.11 mmol) in THF (5 mL) was added DIPEA (0.573 g, 4.44 mmol) at room temperature and stirred for 15 min followed by addition of 4-nitrophenylchloroformate (0.223 g, 1.11 mmol). The reaction mixture was allowed to stir at room temperature for 1 h. Reaction mixture was diluted with water (50 mL) and extracted with ethyl acetate (50 mL×2). The combined organic layer was dried over anhydrous sodium sulphate, filtered and concentrated under reduced pressure. The obtained crude was purified by silica gel (60-120 mesh) column chromatography using 20% ethyl acetate in hexane to afford 4-nitrophenyl (R)-3-(5-methyl-2-oxopyrazin-1(2H)-yl)piperidine-1-carboxylate (0.2 g, Yield: 50.30%) as white solid. LCMS purity: 99.59% (Method A).

### Step-4: Synthesis of (R)—N—((S)-3-(3,4-dihydroisoquinolin-2(1H)-yl)-2-hydroxypropyl)-3-(3,5-dimethyl-2-oxopyrazin-1(2H)-yl)piperidine-1-carboxamide

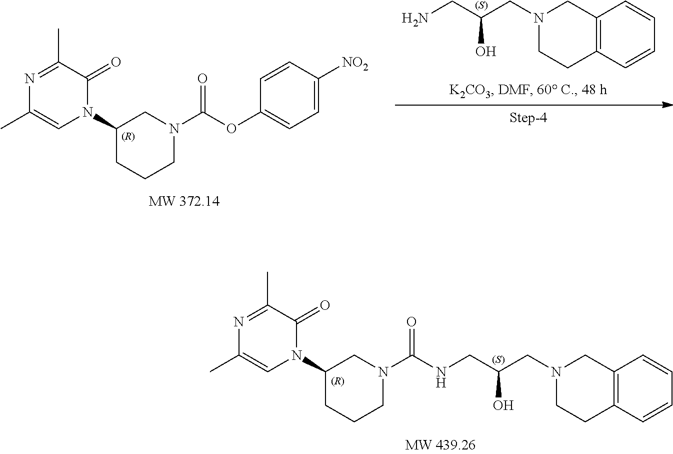

To a stirred solution of 4-nitrophenyl (R)-3-(5-methyl-2-oxopyrazin-1(2H)-yl)piperidine-1-carboxylate (0.1 g, 0.26 mmol) in DMF (2.0 mL) was added Int-A3 (0.066 g, 0.32 mmol) and potassium carbonate (0.148 g, 1.04 mmol) at room temperature. The resulting reaction mixture was heated at 60° C. for 48 h. After completion, the reaction mixture was cooled to room temperature and diluted with ice cold water (100 mL). The resulting suspension was extracted with ethyl acetate (30 mL×2). The combined organic layer was washed with chilled brine (100 mL×3). The organic layer was then dried over anhydrous sodium sulphate, filtered and concentrated under reduced pressure. The obtained crude was purified by reverse phase flash chromatography using 0.1% ammonia solution in water: acetonitrile to afford (R)—N—((S)-3-(3,4-dihydroisoquinolin-2(1H)-yl)-2-hydroxypropyl)-3-(3,5-dimethyl-2-oxopyrazin-1(2H)-yl)piperidine-1-carboxamide (0.05 g, Yield: 40.46%). 1H NMR: 400 MHZ, DMSO-d6: 7.850 (s, 1H), 7.104-7.000 (m, 4H), 6.536-6.510 (m, 1H), 4.925-4.901 (m, 1H), 4.804-4.793 (d, J=4.4 Hz, 1H), 3.722-3.624 (m, 1H), 3.587-3.526 (m, 3H), 3.435-3.385 (m, 1H), 3.280 (br m, 2H), 3.118-3.021 (m, 2H), 2.787-2.714 (m, 2H), 2.701-2.655 (m, 3H), 2.458-2.393 (m, 3H), 2.362 (s, 3H), 2.303 (s, 3H), 1.937-1.928 (m, 1H), 1.720-1.677 (m, 2H), 1.387-1.421 (m, 1H).

### Example 15

### Preparation of N—((S)-3-(3,4-dihydroisoquinolin-2(1H)-yl)-2-hydroxypropyl)-3-(1-methyl-2-oxo-1,2-dihydropyridin-3-yl)piperidine-1-carboxamide

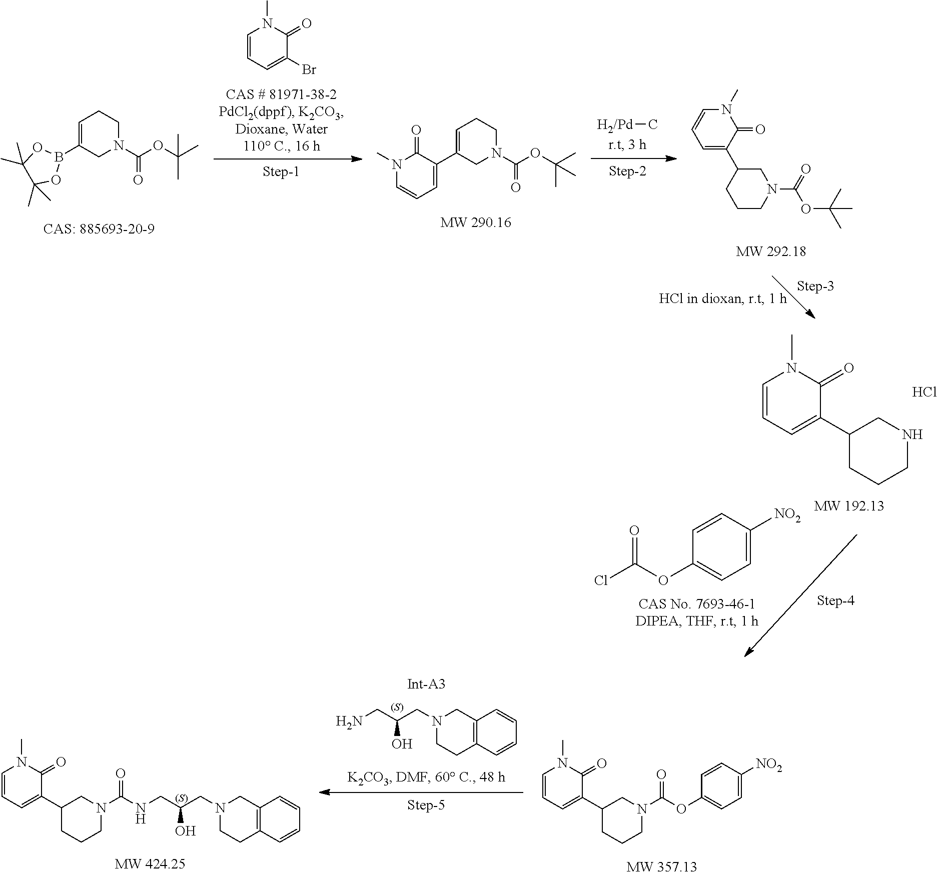

### Synthesis of CAS #81971-38-2

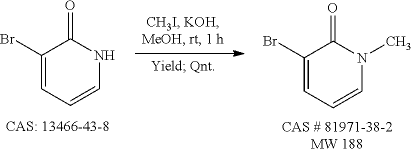

To a stirred solution of CAS No. 1834-27-1 (2 g, 11.4 mmol) in MeOH (20 mL), KOH was (0.9 g, 16.02 mmol) added at room temperature and reaction mixture was allowed to stir for 15 min. CH₃I (7.13 g, 11.4 mmol) was then added and resulting solution was stirred for 1 h at room temperature. After completion the reaction was diluted with water (50 mL) and extracted with DCM (50 mL×2). The combined organic layer was dried over anhydrous sodium sulphate, filtered and concentrated under reduce pressure to afford CAS #81971-38-2 (2 g, qnt) as light yellow sticky solid. LCMS purity: 99.68% (Method A).

### Step-1: Synthesis of tert-butyl 1′-methyl-2′-oxo-1′,2′, 5,6-tetrahydro-[3,3′-bipyridine]-1(2H)-carboxylate

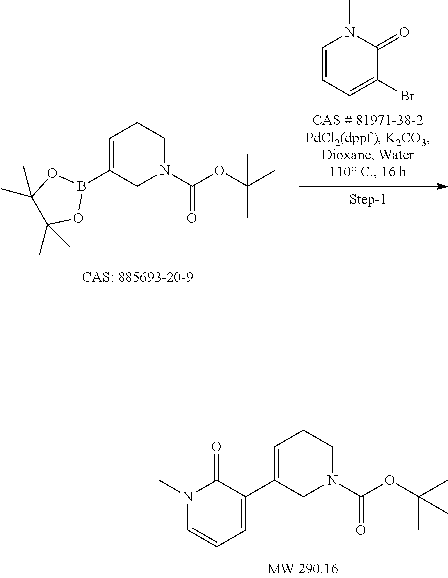

To a stirred solution of CAS #885693-20-9 (1 g, 3.20 mmol) & CAS #81971-38-2 (0.6 g, 3.20 mmol) in 1,4-dioxane (12 mL) and water (2 mL) K₂CO₃ (0.893 g, 6.40 mmol) was added and reaction mixture was degassed using N2 for 15-20 min. PdCl₂(dppf) (0.26 g, 10 mol %) was added and reaction mixture was allowed to stir at 110° C. for 16 h. After completion, the reaction mixture was cooled to room temperature and diluted with water (60 mL). The resulting suspension was extracted with ethyl acetate (60 mL×2). The combined organic layer was dried over anhydrous sodium sulphate, filtered and concentrated under reduce pressure. The obtained crude was purified by silica gel (60-120 mesh) column chromatography using 0.3% MeOH in DCM tert-butyl 1′-methyl-2′-oxo-1′,2′,5,6-tetrahydro-[3,3′-bipyridine]-1(2H)-carboxylate (0.76 g, Yield: 81.57%) as brown sticky liquid. LCMS purity: 83.37% (Method A).

### Step-2: Synthesis of tert-butyl 3-(1-methyl-2-oxo-1,2-dihydropyridin-3-yl)piperidine-1-carboxylate

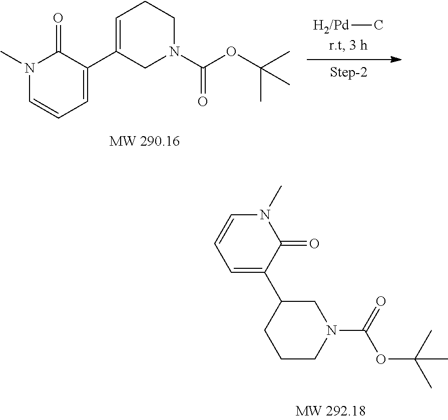

To a stirred solution of tert-butyl 1′-methyl-2′-oxo-1′,2′,5,6-tetrahydro-[3,3′-bipyridine]-1(2H)-carboxylate (0.76 g, 2.60 mmol) in MeOH (10 mL) was added 10% Pd/C (0.076 g, 10% w/w) at room temperature. The reaction mixture bubbled until complete SM consumed on TLC (ca. 3 h). After indicated time reaction mixture was filtered through celite pad, rinsed with MeOH and concentrated under reduced vacuum to afford tert-butyl 3-(1-methyl-2-oxo-1,2-dihydropyridin-3-yl)piperidine-1-carboxylate (0.45 g, Yield: 58.34%) as a brown sticky solid. Crude material was directly used for next step without further purification. LCMS purity: 94.16% (Method A).

### Step-3: Synthesis of 1-methyl-3-(piperidin-3-yl)pyridin-2(1H)-one

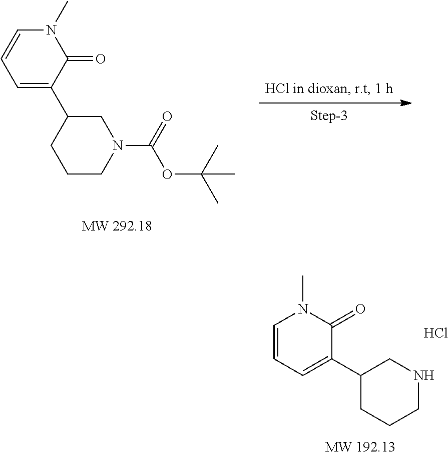 [SMILES: Cn1cccc(C2CCCN(C(=O)OC(C)(C)C)C2)c1=O ; "HCl in dioxan, r.t, 1 h" ; "Step-3" ; Cn1cccc(C2CCCNC2)c1=O ; Cl]

To a stirred solution of tert-butyl 3-(1-methyl-2-oxo-1,2-dihydropyridin-3-yl)piperidine-1-carboxylate (0.45 g, 1.54 mmol) in DCM (5 mL) was added 4N HCl in dioxane (2 mL) at room temperature. The reaction mixture was allowed to stir at room temperature for 1 h. The reaction mixture was concentrated under reduce pressure to afford 1-methyl-3-(piperidin-3-yl)pyridin-2(1H)-one (0.4 g, Yield: Qnt.) as a brown sticky solid. Crude material was directly used for next step without further purification. LCMS purity: 77.38% (Method A).

### Step-4: Synthesis of 4-nitrophenyl 3-(1-methyl-2-oxo-1,2-dihydropyridin-3-yl)piperidine-1-carboxylate

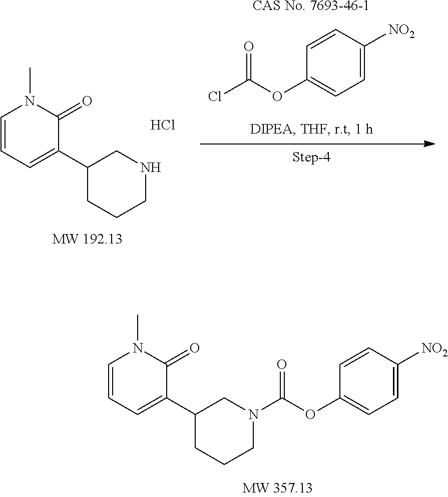

To a stirred solution of tert-butyl 1′-methyl-2′-oxo-1′,2′,5,6-tetrahydro-[3,3′-bipyridine]-1(2H)-carboxylate (0.76 g, 2.60 mmol) in MeOH (10 mL) was added 10% Pd/C (0.076 g, 10% w/w) at room temperature. The reaction mixture bubbled until complete SM consumed on TLC (ca. 3 h). After indicated time reaction mixture was filtered through celite pad, rinsed with MeOH and concentrated under reduced vacuum to afford tert-butyl 3-(1-methyl-2-oxo-1,2-dihydropyridin-3-yl)piperidine-1-carboxylate (0.45 g, Yield: 58.34%) as a brown sticky solid. Crude material was directly used for next step without further purification. LCMS purity: 84.09% (Method A).

### Step-5: Synthesis of N—((S)-3-(3,4-dihydroisoquinolin-2(1H)-yl)-2-hydroxypropyl)-3-(1-methyl-2-oxo-1,2-dihydropyridin-3-yl)piperidine-1-carboxamide

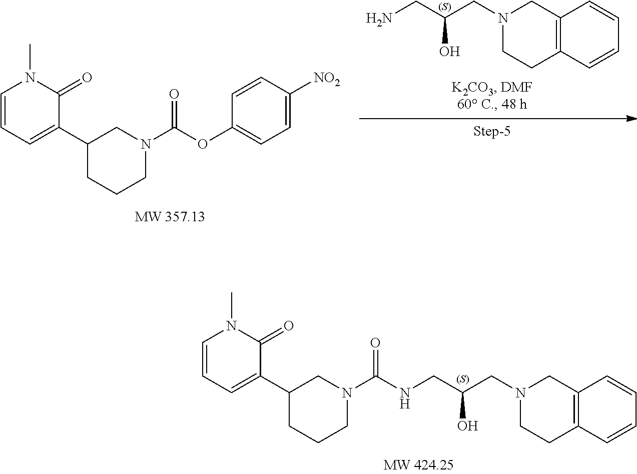

To a stirred solution of 4-nitrophenyl 3-(1-methyl-2-oxo-1,2-dihydropyridin-3-yl)piperidine-1-carboxylate (0.15 g, 0.42 mmol) in DMF (2.0 mL) was added Int-A3 (0.13 g, 0.63 mmol) and potassium carbonate (0.231 g, 1.68 mol) at room temperature. The resulting reaction mixture was heated at 60° C. for 48 h. After completion, the reaction mixture was cooled to room temperature and diluted with ice cold water (100 mL). The resulting suspension was extracted with ethyl acetate (100 mL×2). The combined organic layer was washed with chilled brine (100 mL×3). Organic layer was then dried over anhydrous sodium sulphate, filtered and concentrated under reduced pressure. The obtained crude was purified by reverse phase flash chromatography using 0.1% ammonia solution in water:acetonitrile to afford N—((S)-3-(3,4-dihydroisoquinolin-2(1H)-yl)-2-hydroxypropyl)-3-(1-methyl-2-oxo-1,2-dihydropyridin-3-yl)piperidine-1-carboxamide (0.03 g, Yield: 16.85%). 1H NMR: 400 MHZ, DMSO-d6: 7.601-7.580 (m, 1H), 7.251-7.218 (m, 1H), 7.109-7.027 (m, 4H), 6.565 (br, 1H), 6.205-6.171 (t, J=6.8 Hz, 1H), 4.899 (br, 1H), 3.930-3.902 (m, 2H), 3.791 (br s, 1H), 3.632-3.613 (m, 2H) 3.440 (s, 3H), 3.212-3.179 (m, 1H), 3.166-3.060 (m, 1H), 2.804-2.652 (m, 6H), 1.602-1.571 (m, 1H), 1.509-1.501 (m, 1H), 1.471-1.440 (m, 1H).

### Example 16

### Preparation of (R)—N—((S)-3-(3,4-dihydroisoquinolin-2(1H)-yl-1,1,4,4-d4)-2-hydroxypropyl)-3-(3,5-dimethyl-6-oxopyridazin-1(6H)-yl)piperidine-1-carboxamide

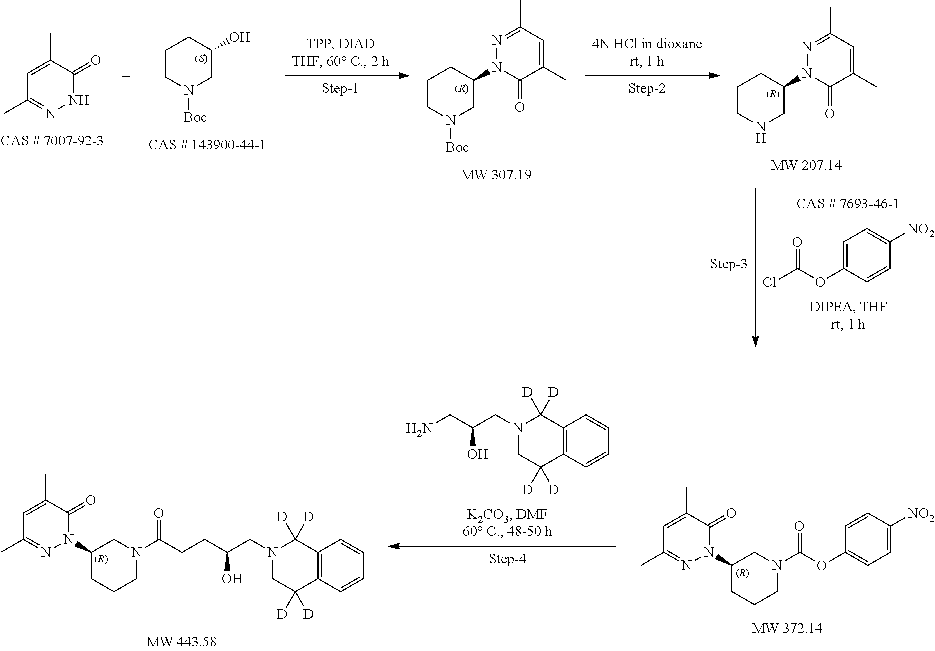

### Synthesis of CAS #7007-92-3 (4,6-dimethylpyridazin-3(2H)-one)

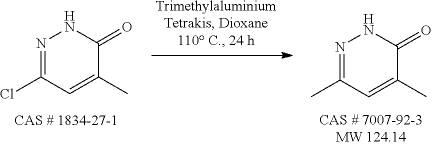

To a stirred solution of CAS No. 1834-27-1 (1.0 g, 6.94 mmol) in 1,4-dioxane (7 mL), tetrakis(triphenylphosphine)palladium (0.23 g, 0.25 mmol) was added followed by trimethyl aluminum (1M in toluene, 4 mL, 13.88 mmol) under nitrogen atmosphere. The reaction mixture was allowed to stir at 100° C. for 24 h. After completion, the reaction mixture was cooled to room temperature and diluted with water (50 mL). The resulting suspension was extracted with ethyl acetate (50 mL×2). The combined organic layer was dried over anhydrous sodium sulphate, filtered and concentrated under reduce pressure. The obtained crude was purified by silica gel (60-120 mesh) column chromatography using 55% ethyl acetate in Hexane to afford 4,6-dimethylpyridazin-3(2H)-one (0.8 g, Yield: 93.16%) as light yellow solid. LCMS purity: 42.90% (Method A).

### Step-1: Synthesis of tert-butyl (R)-3-(3,5-dimethyl-6-oxopyridazin-1(6H)-yl)piperidine-1-carboxylate

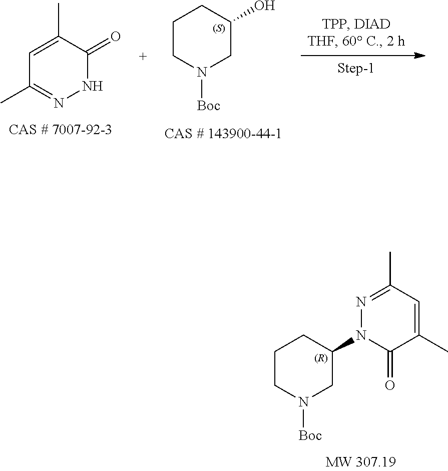

To a stirred solution of CAS No. 7007-92-3 (0.493 g, 3.98 mmol) and CAS No. 143900-44-41 (0.8 g, 3.98 mmol) in dry THF (8.0 mL), TPP (1.56 g, 5.97 mmol) was added. The reaction mixture was allowed to stir at 60° C. for 30 min, after that DIAD (1.20 g, 5.97 mmol) was added dropwise over period of 10 min. The reaction mixture was allowed to stir at 60° C. for 2 h. After completion, the reaction mixture was cooled to room temperature and diluted with water (50 mL). The resulting suspension was extracted with ethyl acetate (50 mL×2). The combined organic layer was dried over anhydrous sodium sulphate, filtered and concentrated under reduce pressure. The obtained crude was purified by silica gel (60-120 mesh) column chromatography using 12% ethyl acetate in hexane to afford tert-butyl (R)-3-(3,5-dimethyl-6-oxopyridazin-1(6H)-yl)piperidine-1-carboxylate (0.33 g, Yield: 24.73%) as brown sticky liquid. LCMS purity: 90.88% (Method A).

### Step-2: Synthesis of (R)-4,6-dimethyl-2-(piperidin-3-yl)pyridazin-3(2H)-one

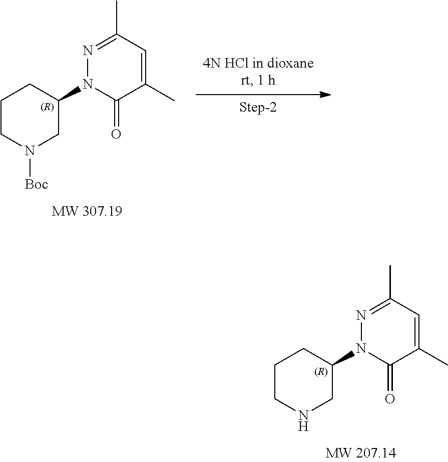

To a stirred solution of tert-butyl (R)-3-(3,5-dimethyl-6-oxopyridazin-1(6H)-yl)piperidine-1-carboxylate (0.3 g, 0.97 mmol) in DCM (2.0 mL) was added 4N HCl in dioxane (2.0 mL) at room temperature. The reaction mixture was allowed to stir at room temperature for 30 min. The reaction mixture was concentrated under reduce pressure to afford (R)-4,6-dimethyl-2-(piperidin-3-yl)pyridazin-3(2H)-one (0.28 g, Yield: Qnt.) as a white sticky solid. Crude material was directly used for next step without further purification. LCMS purity: 93.37% (Method A).

### Step-3: Synthesis of 4-nitrophenyl (R)-3-(3,5-dimethyl-6-oxopyridazin-1(6H)-yl)piperidine-1-carboxylate

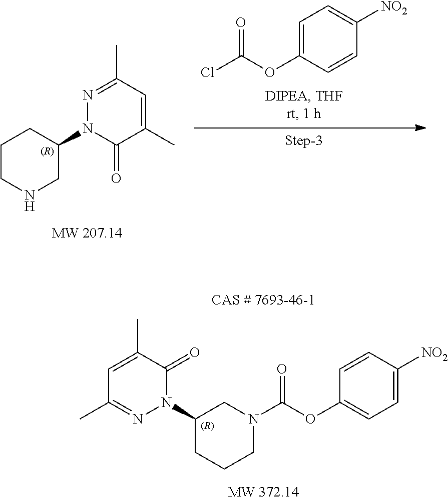

To a stirred solution of (R)-4,6-dimethyl-2-(piperidin-3-yl)pyridazin-3(2H)-one (0.28 g, 1.35 mmol) in THF (3.0 mL) was added DIPEA (0.697 g, 5.40 mmol) at room temperature and stirred for 15 min followed by addition of 4-nitrophenylchloroformate (0.271 g, 1.35 mmol). The reaction mixture was allowed to stir at room temperature for 30 min. The reaction mixture was diluted with water (50 mL) and extracted with ethyl acetate (50 mL×2). The combined organic layer was dried over anhydrous sodium sulphate, filtered and concentrated under reduced pressure. The obtained crude was purified by silica gel (60-120 mesh) column chromatography using 20% Ethyl acetate in Hexane to afford 4-nitrophenyl (R)-3-(3,5-dimethyl-6-oxopyridazin-1(6H)-yl)piperidine-1-carboxylate (0.0.21 g, Yield: 38.96%) as white solid. LCMS purity: 96.80% (Method A).

### Step-4: Synthesis of (R)—(R)—N—((S)-3-(3,4-dihydroisoquinolin-2(1H)-yl-1,1,4,4-d4)-2-hydroxypropyl)-3-(3,5-dimethyl-6-oxopyridazin-1(6H)-yl)piperidine-1-carboxamide

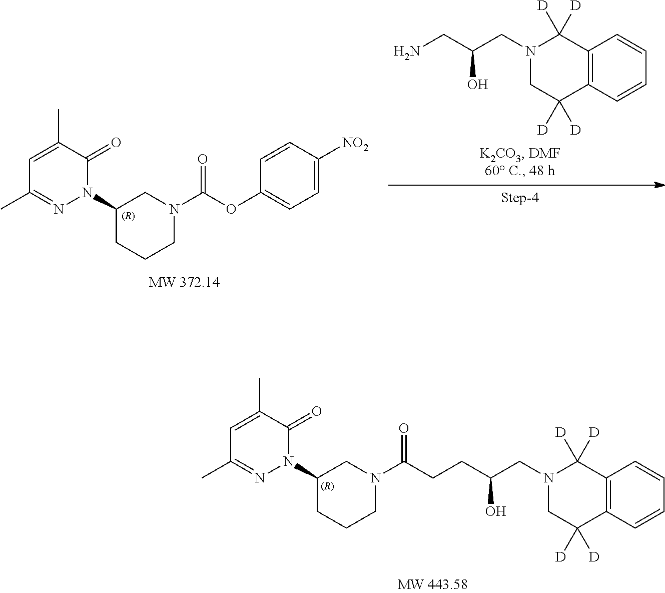

To a stirred solution of 4-nitrophenyl (R)-3-(3,5-dimethyl-6-oxopyridazin-1(6H)-yl)piperidine-1-carboxylate (0.08 g, 0.24 mmol) in DMF (1.5 mL) was added Int-C₃ (0.054 g, 0.25 mmol) and potassium carbonate (0.118 g, 0.86 mmol) at room temperature. Reaction mixture was then heated at 60° C. for 48 h. After completion, the reaction mixture was cooled to room temperature and diluted with ice cold water (50 mL). The resulting suspension was extracted with ethyl acetate (50 mL×2). The combined organic layer was washed with chilled brine (60 mL×3). Organic layer was dried over anhydrous sodium sulphate, filtered and concentrated under reduced pressure. The obtained crude was purified by prep HPLC using 0.1% formic solution in water:acetonitrile to afford (R)—N—((S)-3-(3,4-dihydroisoquinolin-2(1H)-yl-1, 1,4,4-d4)-2-hydroxypropyl)-3-(3,5-dimethyl-6-oxopyridazin-1(6H)-yl)piperidine-1-carboxamide (0.012 g, Yield: 12.59%). 1H NMR: 400 MHZ, DMSO-d6: 7.221 (s, 1H), 7.107-7.021 (m, 4H), 6.677-6.651 (m, 1H), 4.818 (br s, 1H), 4.676-4.654 (m, 1H), 4.891-4.751 (m, 1H), 4.699-4.608 (m, 1H), 4.011-3.853 (m, 2H), 3.799-3.783 (m, 1H), 3.342-3.207 (m, 3H), 3.175-2.975 (m, 2H), 2.907-2.876 (m, 2H), 2.228 (s, 3H), 2.065 (s, 3H), 1.812-1.801 (m, 1H), 1.687 (br, 1H), 1.654 (br, 1H), 1.238 (s, 1H).

### Biological Activity of Compounds

By way of comparison, the compound from Example 5A of WO 2018/167269 (see below) was prepared as described in this document. This compound is referred to below as compound CEx.

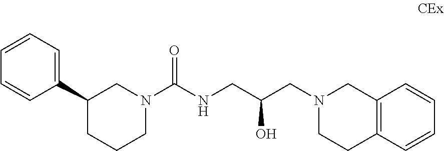 [SMILES: O=C(NC[C@H](O)CN1CCc2ccccc2C1)N1CCC[C@@H](c2ccccc2)C1]

### Methods

The activity of the compounds was tested using several industry accepted methods. These included methods to measure the human hepatocyte clearance values (Human Hep Clint) and a PRMT5 assay. The methods that were used were conventional.

### Hepatocyte Stability Assay

### Reagents

### Cryopreserve Hepatocytes

Cell viability was tested by trypan blue exclusion after thawing. Results are showed in the table below.

| | Cell | | | Lot | | Vials |
|---|---|---|---|---|---|---|
| Species | Viability | Vendor | Cat No. | No. | Donor | Used |
| Human | 89.7% | Biorec- | X008001 | AEL | Pooled of | 3 |
| | | lamation | | | 10 Human | |
| | | IVT | | | Donors | |
| | | | | | (Mixed | |
| | | | | | Gender) | |

The control compound information is shown in the table below.

| Compound ID | Source | Cat# | Molecular Weight |
|---|---|---|---|
| 7-Ethoxycoumarin | Sigma | E1379 | 190.2 |
| 7-Hydroxycoumarin | Sigma | H24003 | 162.1 |

### Buffer Solution Information

Thawing Medium: Williams' Medium E containing 5% fetal bovine serum and 30% Percoll solution and other supplements.

Incubation Medium: Williams' Medium E (no phenol red) containing 2 mM L-Glutamine and 25 mM HEPES.

Stop Solution: Acetonitrile containing 200 ng/ml tolbutamide and labetalol as internal standards.

Dilution Solution: Ultra-pure water.

### Experimental Procedure

The following steps were performed.
1. 10 mM test compounds were provided by Compound Sample Group.
2. 30 mM positive control stock solutions: Dissolved accurate amount of positive control compounds in dimethyl sulfoxide (DMSO).
3. 1000× Stock Solution: Diluted 10 mM test compounds and 30 mM positive control compounds to 1 mM and 3 mM with DMSO in 96-well plates.
4. 100× Dosing Solution: Diluted 1 mM test compounds and 3 mM positive control compounds to 100 UM and 300 UM dosing solutions with ACN.
5. Preparation of 0.5×106/mL cells suspension: cryopreserved cells were thawed, isolated and suspended in Incubation Medium, then diluted with pre-warmed Incubation Medium to 0.5×106 cells/mL.
6. Add 198 μL of pre-warmed cell suspensions in 96-well plates.
7. Preparation of Quenching Plate: Transfer 125 μL of stop solution (acetonitrile containing 200 ng/ml tolbutamide and 200 ng/ml labetalol as internal standards) in a set of pre-labelled 96-well plates.
8. Spike 2 μL dosing solution to each well of 96-well plates in duplicates.
9. For T0 Samples, mix to achieve a homogenous suspension for about 1 min, then immediately transfer 25 μL of each sample into well containing 125 μL of ice-cold stop solution followed by mixing.
10. Incubate all plates at 37° ° C. in a 95% humidified incubator at 5% CO2 to start the reactions with constant shaking at about 600 rpm.
11. At 15, 30, 60 and 90 min, mix samples and then transfer 25 μL of each sample at each time point to well containing 125 μL of ice-cold stop solution followed by mixing.
12. Medium Control (MC) sample plates (labelled as T0-MC and T90-MC) are prepared at TO and T90 by adding the same components to each well except cell suspensions.
13. At each corresponding time point, stop the reactions by removing the plates from incubator and mixing with 125 μL of ice-cold stop solution.
14. Vortex the plates immediately on a plate shaker at 500 rpm for 10 minutes. Then, centrifuge all sample plates at 3220× g for 20 min at 4° C.
15. After centrifugation, 80 μL/well of supernatant in the sample plates are transferred to another set of pre-labelled 96-well plates which containing 240 μL of ultra pure water according to the plate map.
16. Analytical plates are sealed and store at 4° C. until LC-MS/MS analysis.

### The Final Concentration of Each Component in the Incubation Medium

| Component | Final Concentration | |
|---|---|---|
| Hepatocyte | 0.5 × 10⁶ | cells/mL |
| Test Compound | 1 | μM |
| Positive Compound | 3 | μM |

| | |
|---|---|
| ACN | 0.90% |
| DMSO | 0.10% |

### Data Analysis

The remaining percents of test articles after incubation were calculated by the follow equations:

$${C}_{t} = {C}_{0} \cdot {e}^{- kt}$$

$$when$$

$${C}_{t} = \frac{1}{2} {C}_{0} , {t}_{1 / 2} = \frac{ln 2}{k} = \frac{0.693}{k} .$$

Use equation of first order kinetics to calculate t_{1/2} and CLᵢₙₜ. Equation of first order kinetics:

$${C}_{t} = {C}_{0} \cdot {e}^{- kt}$$

$$when {C}_{t} = \frac{1}{2} {C}_{0} , {t}_{1 / 2} = \frac{ln 2}{k} = \frac{0.693}{k} .$$

CL_{int (hep)}=k/million cells per mL

CL_{int (liver)}=CL_{int (hep)}·liver weight (g/kg body weight)*hepatocellularity

### PRMT5 Assay

A PRMT5 chemiluminescent assay was used to measure the IC50 activity of PRMT5 of the compounds of CEx and Examples 1 to 8 above. Biotinylated histone peptides were synthesized and attached to 384-well plates. Compound serial dilutions were performed and added to the assay plate. Histone H4 monomethyl R³ antibody was obtained from Abcam. A master mix for each well was prepared and human PRMT5/MEP50 (expressed in HEK293 cells) diluted in assay buffer to a concentration of 5 ng/μL. The reaction was incubated and slowly rotated for 60 minutes at the point of PRMT5/MEP50 addition. The supernatant from the wells was removed and blocking buffer was added to each well and rotated for 10 minutes. The primary antibody was diluted and added to every well for 60 minutes, before it was removed and the wells washed. The horse radish peroxidase (HRP)-coupled secondary antibody was diluted and added to each well with an incubation time of 30 minutes. The HRP chemiluminescent substrate was added to every well. The plate was read on a Flourstar Omega BMG Labtech instrument (Ortenberg, Germany) and the analysis of IC50 was performed using the Flourstar Omega BMG Labtech software.

### Results

The results for compound CEx and the compounds of Examples 1 to 8 are shown in Table 1.

TABLE 1

| | | Human Hep Clint | PRMT5 |
|---|---|---|---|
| | cLogP | (μL/min/10⁶ cells) | pIC₅₀ |
| CEx | 3.17 | 54.1 | 7.9 |
| Ex. 1 | 1.02 | 1.7 | 7.3 |
| Ex. 2 | 1.36 | <3 | 7.8 |
| Ex. 3 | 1.63 | 2.5 | 7.5 |
| Ex. 4 | 1.95 | 2.5 | 7.5 |
| Ex. 5 | 1.55 | 0 | 7.7 |
| Ex. 6 | 1.63 | <6 | 7.9 |
| Ex. 7 | 1.04 | 10.8 | 8.1 |
| Ex. 8 | 1.23 | <6.4 | 7.9 |

### REFERENCES

Chung, J. et al., “Protein arginine methyltransferase 5 (PRMT5) inhibition induces lymphoma cell death through reactivation of the retinoblastoma tumor suppressor pathway and polycomb repressor complex 2 (PRC2) silencing”, J. Biol. Chem. 288, 35534-35547 (2013).
Wei, L. et al., “Protein arginine methyltransferase 5 is a potential oncoprotein that upregulates G1 cyclins/cyclin-dependent kinases and the phosphoinositide 3-kinase/AKT signaling cascade”, Cancer Sci. 103, 1640-1650 (2012).
Powers, M. A. et al., “Protein arginine methyltransferase 5 accelerates tumor growth by arginine methylation of the tumor suppressor programmed cell death 4”, Cancer Res. 71, 5579-5587 (2011).
Cho, E. C et al., “Arginine methylation controls growth regulation by E2F1”, EMBO J. 31, 1785-1797 (2012).
Pal, S. et al., “Low levels of miR-92b/96 induce PRMT5 translation and H3R8/H4R3 methylation in mantle cell lymphoma”, EMBO J. 26, 3558-3569 (2007).
Elayne, C. P et al., “Selective inhibitor of PRMT5 with in vivo and in vitro potency in MCL models”, Nature chemical biology. 11, 432-437 (2015).
Stopa, N. et al., “The PRMT5 arginine methyltransferase: many roles in development, cancer and beyond”, Cell Mol Life Sci., 72(11), 2041-59 (2015 June).
Chen, H. et al., “A TGFß-PRMT5-MEP50 axis regulates cancer cell invasion through histone H3 and H4 arginine methylation coupled transcriptional activation and repression”, Oncogene, 36(3), 373-386 (2017).
Kryukov, G. V. et al., “MTAP deletion confers enhanced dependency on the PRMT5 arginine methyltransferase in cancer cells”, Science, 351(6278), 1214-1218 (2016).
Jin, Y. et al., “Targeting methyltransferase PRMT5 eliminates leukemia stem cells in chronic myelogenous leukemia”, J. Clin. Invest., 126(10), 3961-3980 (2016).
Banasavadi-Siddegowda, Y. K. et al., “PRMT5-PTEN molecular pathway regulates senescence and self-renewal of primary glioblastoma neurosphere cells”, Oncogene, 36(2), 263-274 (2017).
Yang, Y. et al., “Protein arginine methyltransferases and cancer”, Nature Reviews Cancer, 13(1), 37-50 (2012).
Kumar, B. et al., “High Expression of PRMT5 and Cyclin D1 Is Associated With Poor Outcome in Oropharyngeal Squamous Cell Carcinoma (OPSCC) Patients and Is Inversely Associated With p16 Status”, International Journal of Radiation Oncology, 88(2), 513-514 (2014).
Karkhanis, V. et al., “Versatility of PRMT5-induced methylation in growth control and development”, Trends Biochem Sci, 36(12), 633-641 (2011).
Zhang, H. T. et al., “Transcriptional activation of PRMT5 by NF—Y is required for cell growth and negatively regulated by the PKC/c-Fos signaling in prostate cancer cells”, Biochim Biophys Acta, 1839(11), 1330-1340 (2014).
Powers, M. A. et al., “Protein arginine methyltransferase 5 accelerates tumor growth by arginine methylation of the tumor suppressor programmed cell death”, Cancer Res, 71(16), 5579-5587 (2011).
Yan, F. et al., “Genetic validation of the protein arginine methyltransferase PRMT5 as a candidate therapeutic target in glioblastoma”, Cancer Res, 74(6), 1752-1765 (2014).
Ibrahim, R. et al., “Expression of PRMT5 in lung adenocarcinoma and its significance in epithelial-mesenchymal transition”, Hum Pathol, 45(7), 1397-1405 (2014).
Gu, Z. et al., “Protein arginine methyltransferase 5 is essential for growth of lung cancer cells”, Biochem J, 446(2), 235-241 (2012).
Yang, F. et al, “Proliferative role of TRAF4 in breast cancer by upregulating PRMT5 nuclear expression”, Tumour Biol, 36(8), 5901-5911 (2015).
Pak, M. G. et al., “High nuclear expression of protein arginine methyltransferase-5 is a potentially useful marker to estimate submucosal invasion in endoscopically resected early colorectal carcinoma”, Pathol Int, 65(10), 541-548 (2015).
Gu, Z. et al., “Protein arginine methyltransferase 5 functions in opposite ways in the cytoplasm and nucleus of prostate cancer cells”, PLOS One, 7(8), e44033 (2012).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g. “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

What is claimed is:

1. A compound of formula (1) or a deuterated form, salt, solvate, or hydrate thereof, 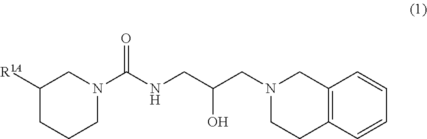
wherein:
R^{1A} is represented by formula (A1) or (A1′), 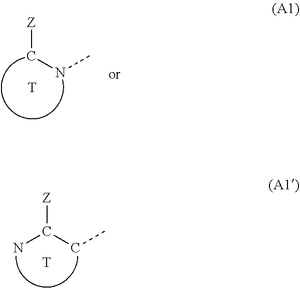
Z is ═O;
T taken together with the intervening carbon and nitrogen atoms (e.g. shown in formula (A1)) is selected from a monocyclic 5- to 7-membered heterocycloalkyl group, a fused bicyclic 6- to 10-membered heterocycloalkyl group and a bridged bicyclic 6- to 9-membered heterocycloalkyl group, wherein each of the monocyclic 5- to 7-membered heterocycloalkyl group, the fused bicyclic 6- to 10-membered heterocycloalkyl group and the bridged bicyclic 6- to 9-membered heterocycloalkyl group is optionally substituted with one or more R^{S1};
R^{S1} is selected from C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
R^{S2} is selected from hydroxy, halo, CN and nitro.

2. The compound according to claim 1 or a deuterated form, salt, solvate or hydrate thereof, wherein RIA is represented by formula (A2),

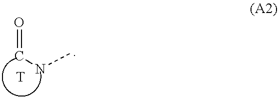

3. The compound according to claim 1, or a deuterated form, salt, solvate or hydrate thereof, wherein the monocyclic 5- to 7-membered heterocycloalkyl group is a monocyclic 5- or 6-membered heterocycloalkyl group optionally substituted with one or more R^{S1}.

4-5. (canceled)

6. The compound according to claim 1 or a deuterated form, salt, solvate or hydrate thereof, wherein the fused bicyclic 6- to 10-membered heterocycloalkyl group has a first 5- or 6-membered ring including the intervening carbon and nitrogen atoms and a second ring fused to the first 5- or 6-membered ring, and wherein each of the first 5- or 6-membered ring and the second ring is optionally substituted with one or more R^{S1}.

7. The compound according to claim 6 or a deuterated form, salt, solvate or hydrate thereof, wherein the second ring is a 3- to 6-membered ring optionally substituted with one or more R^{S1}.

8-11. (canceled)

12. The compound according to claim 1 or a deuterated form, salt, solvate or hydrate thereof, wherein each R^{S1} is selected from C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.

13. The compound according to claim 12 or a deuterated form, salt, solvate or hydrate thereof, wherein each R^{S1} is selected from C₁₋₆alkyl, C₁₋₆alkoxy, C₃₋₁₂cycloalkyl, hydroxy and chloro, wherein the C₁₋₆alkyl and the C₃₋₁₂cycloalkyl is each optionally substituted with one or more R^{S2}.

14. The compound according to claim 12 or a deuterated form, salt, solvate or hydrate thereof, wherein each R^{S1} is selected from unsubstituted C₁₋₆alkyl, fluoro-C₁₋₆alkyl, fluoro and chloro.

15. The compound according to claim 1 or a deuterated form, salt, solvate or hydrate thereof, wherein R^{1A} is represented by formula (B1), 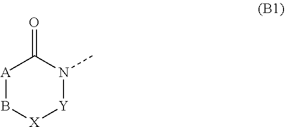
(a) X—Y is selected from CR^{3A}═CR^{2A}, CR^{3A}—N, N═CR^{2A}, N═N, C(R^{3A})₂—C(R^{2A})₂, C(R^{3A})₂—NR^{2A}, NR^{3A}—C(R^{2A})₂, NR^{3A}—NR^{2A}, C(R^{3A})₂—O, O—C(R^{2A})₂ C(R^{3A})₂—S and S^{A}—C(R^{2A})₂; and
A-B is selected from CR^{5A}═CR^{4A}, CR^{5A}═N, N═CR^{4A}, N═N, C(R^{5A})₂—C(R^{4A})₂, C(R^{5A})₂—NR^{4A}, NR^{5A}—C(R^{4A})₂, NR^{5A}—NR^{4A}, C(R^{5A})₂—O, O—C(R^{4A})₂, C(R^{5A})₂—S and S—C(R^{4A})₂; or
(b) A is selected from C(R^{5A})₂, NR^{5A}, O and S;
Y is selected from C(R^{2A})₂ and NR^{2A}; and
B—X is selected from CR^{4A}═CR^{3A}, CR^{4A}═N and N═CR^{3A};
wherein each R^{2A}, R^{3A}, R^{4A} and R^{5A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
each R^{S2} is selected from hydroxy, halo, CN and nitro.

16. The compound according to claim 15 or a deuterated form, salt, solvate, or hydrate thereof, wherein R^{1A} is represented by one of the following formulae,

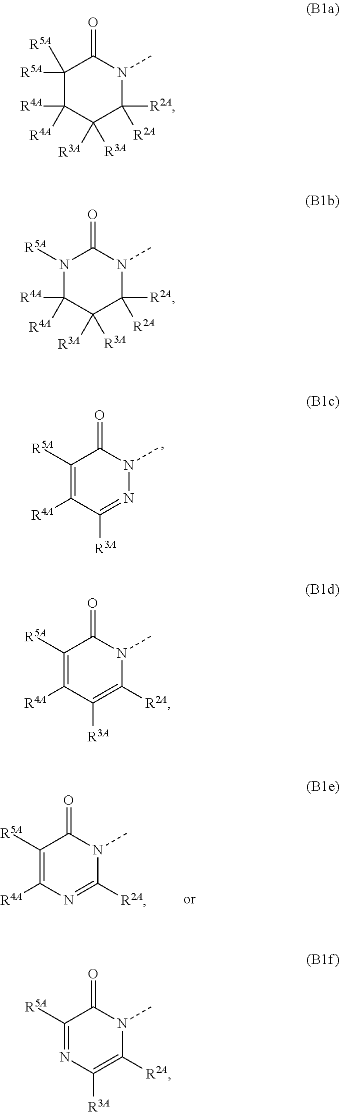

17. The compound according to claim 16 or a deuterated form, salt, solvate, or hydrate thereof, wherein R^{1A} is represented by one of the following formulae:

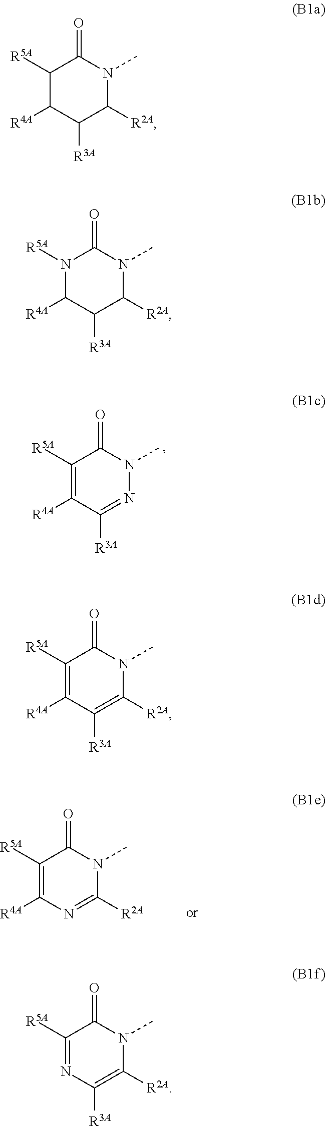

18. The compound according to claim 15 or a deuterated form, salt, solvate, or hydrate thereof, wherein each R^{2A}, R^{3A}, R^{4A} and R^{5A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.

19. The compound according to claim 18 or a deuterated form, salt, solvate or hydrate thereof, wherein each R^{2A}, R^{3A}, R^{4A} and R^{5A} is the same or different and is independently selected from hydrogen, unsubstituted C₁₋₆alkyl, halo-C₁₋₆alkyl, fluoro and chloro.

20. The compound according to claim 1 or a deuterated form, salt, solvate or hydrate thereof, wherein RIA is represented by formula (C1), 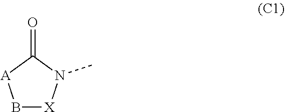
wherein either:
(a) X is selected from C(R^{2A})₂ and NR^{2A}; and
A-B is selected from CR^{4A}═CR^{3A}, CR^{4A}═N, N═CR^{3A}, N═N, C(R^{4A})₂—C(R^{3A})₂, C(R^{4A})₂—NR^{3A}, NR^{4A}—C(R^{3A})₂, NR^{4A}—NR^{3A}, C(R^{4A})₂—O, O—C(R^{3A})₂, C(R^{4A})₂—S and S—C(R^{3A})₂; or
(b) A is selected from C(R^{4A})₂, NR^{4A}, O and S; and
B—X is selected from CR^{3A}═CR^{2A}, CR^{3A}═N, N═CR^{2A} and N═N;
wherein each R^{2A}, R^{3A}, R^{4A} and R^{5A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
each R^{S2} is selected from hydroxy, halo, CN and nitro.

21. The compound according to claim 20 or a deuterated form, salt, solvate, or hydrate thereof, wherein R^{1A} is represented by formula (C3), 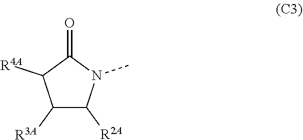
wherein R^{2A}, R^{3A} and R^{4A} is each independently selected from hydrogen, C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
each R^{S2} is selected from hydroxy, halo, CN and nitro.

22. The compound according to claim 20 or a deuterated form, salt, solvate, or hydrate thereof, wherein each R^{2A}, R^{3A} and R^{4A} is the same or different and is independently selected from hydrogen, C₁₋₆alkyl, C₂₋₆alkenyl, C₂₋₆alkynyl, C₁₋₆alkoxy, C₃₋₁₂cycloalkyl, hydroxy, chloro, bromo, fluoro, CN and nitro, wherein the C₁₋₆alkyl, the C₂₋₆alkenyl, the C₂₋₆alkynyl and the C₃₋₁₂cycloalkyl is each optionally substituted with one or more R^{S2}.

23. The compound according to claim 22 or a deuterated form, salt, solvate or hydrate thereof, wherein each R^{2A}, R^{3A} and R^{4A} is the same or different and is independently selected from hydrogen, unsubstituted C₁₋₆alkyl, halo-C₁₋₆alkyl, fluoro and chloro.

24. The compound according to claim 1 selected from: 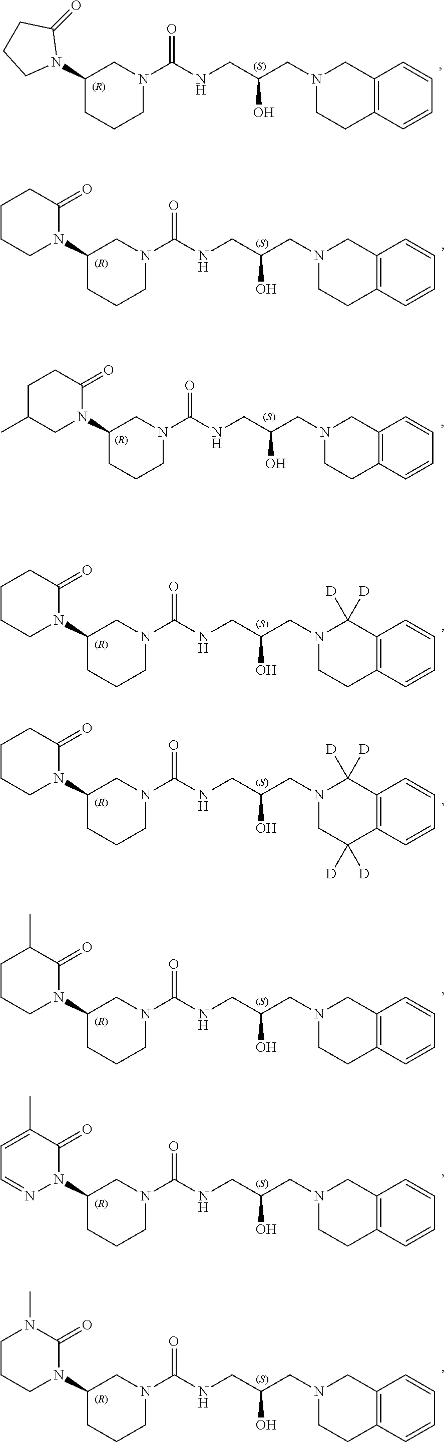 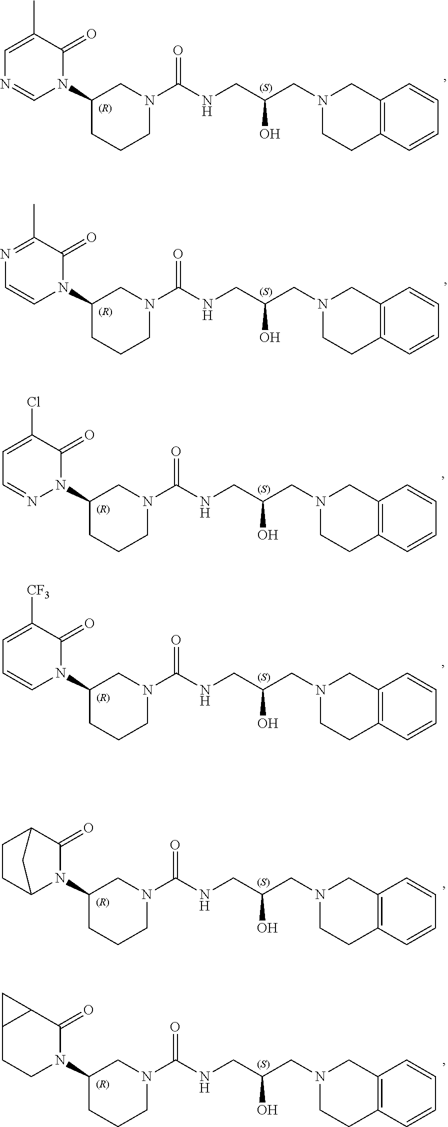 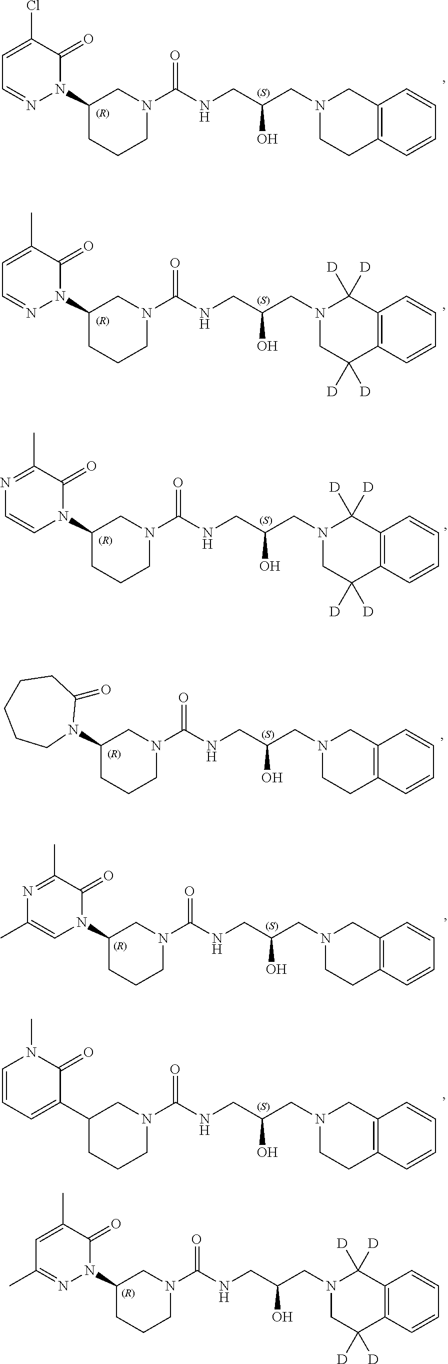
or a salt, solvate, or hydrate thereof.

25. A pharmaceutical composition which comprises a compound according to claim 1, or a deuterated form, pharmaceutically acceptable salt, hydrate or solvate thereof, and optionally a pharmaceutically acceptable excipient.

26. (canceled)

27. A method of treating or preventing a PRMT5-mediated disorder,
said method comprising administering to a subject in need thereof an effective amount of a compound according to claim 1, or a deuterated form, pharmaceutically acceptable salt, hydrate or solvate thereof.

28-31. (canceled)
